(19) United States
(12) Patent Application Publication
(10) Pub. No.: US 2025/0382302 A1
(43) Pub. Date: Dec. 18, 2025

(54) Anticancer polyheterocyclic compounds and associated methods

(71) Applicant: University of Denver, Denver, CO (US)

(72) Inventors: Andrei G. Kutateladze, Denver, CO (US); Dmitry M. Kuznetsov, Willow Grove, PA (US); Malla Reddy Gannarapu, Denver, CO (US); Srinivas Beduru, Denver, CO (US)

(21) Appl. No.: 19/240,605

(22) Filed: Jun. 17, 2025

Related U.S. Application Data
(60) Provisional application No. 63/661,432, filed on Jun. 18, 2024.

(51) Int. Cl. C07D 491/048; A61K 31/167; A61K 31/341; A61K 31/381; A61K 31/4166; A61K 31/4184; A61K 31/4188; A61K 31/4985; C07C 233/73; C07D 207/327; C07D 307/36; C07D 333/20; C07D 471/14; C07D 487/04; C07D 487/14; C07D 491/08; C07D 495/06

(52) U.S. Cl. CPC C07D 491/048; A61K 31/167; A61K 31/341; A61K 31/381; A61K 31/4166; A61K 31/4184; A61K 31/4188; A61K 31/4985; C07C 233/73; C07D 207/327; C07D 307/36; C07D 333/20; C07D 471/14; C07D 487/04; C07D 487/14; C07D 491/08; C07D 495/06

(57) ABSTRACT

A method of building complex molecules based on a complexity building photoinduced cascade approach is described herein. Additionally, complex molecules with highly complex molecular structures enabled by the complexity building photoinduced cascade approach are described herein. As disclosed herein, the molecules may be synthesized for anticancer properties.

### PRIORITY CLAIM

This application claims the benefit of U.S. Provisional Patent Application No. 63/661,432, filed Jun. 18, 2024, entitled “Anticancer Polyheterocyclic Compounds and Associated Methods,” the entirety of which is hereby incorporated by reference.

### SPONSORED RESEARCH AND DEVELOPMENT

This invention was made with government support under CHE-2247411 awarded by National Science Foundation. The government has certain rights in the invention.

### FIELD OF THE INVENTION

The present invention relates to complex molecules and synthesis thereof. In particular, but not by way of limitation, the present invention relates to polyheterocyclic compounds and synthesis thereof.

### DESCRIPTION OF RELATED ART

Quinone-based organic molecules play important roles in developing therapeutic drugs, agrochemicals, and functional materials and acting as building blocks in total synthesis. Notably, in benzoquinone derivatives incorporated with heteroatoms such as nitrogen or oxygen, the redox potential of the innate carbonyl groups increases, thus significantly enhancing the biological activities of the resulting heterocycles.

Abundant and easily accessible, aromatic compounds with stabilized cyclic n-electron systems, such as benzene, naphthalene, pyridine, and quinoline, serve as natural feedstocks. Commonly utilized in organic synthesis are the well-established synthetic transformations that involve introducing and manipulating functional groups on aromatic rings, making a diverse range of substituted aromatics prevalent as starting materials. This context has spurred considerable interest in dearomatization reactions, aiming to swiftly construct complex, non-planar scaffolds from planar aromatic compounds.

A primary challenge in achieving such transformations lies in overcoming the inherent stabilization of aromatic systems. Notably, recent advancements have introduced sustainable approaches to dearomatization, leveraging visible light-induced photochemical methods. These methods, involving energy or electron transfer, have been successfully applied to numerous aromatic feedstocks, including arenes, phenols, indoles, heteroarenes, and more.

Despite recent strides in the development of dearomatization strategies for synthesizing valuable organic molecules, the task of selectively converting benzene rings into the corresponding saturated or partially saturated cyclic carbon skeletons under mild reaction conditions remains exceptionally challenging.

Thus, there is a need for an improved method of synthesizing such complex molecules as well as for the resulting complex molecules with unique structures enabled by such improved synthesis methods.

### SUMMARY OF THE INVENTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

In an embodiment, a method of building complex molecules based on a complexity building photoinduced cascade approach is described.

In a further embodiment, complex molecules with highly complex molecular structures enabled by the complexity building photoinduced cascade approach are described.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION OF THE INVENTION

The invention includes a synthesis approach based on complexity building photoinduced cascade and complex molecules synthesized using this approach. Existing methods do not allow for synthesis of such complex molecules with the reduced number of synthesis steps enabled by the presently described approach. By taking advantage of a modular design of photo-precursors, the present approach allows a photoinduced multi-step cascade approach, leading to considerable growth of molecular complexity in very few, experimentally simple steps.

It is understood by a person of ordinary skill in the art that substitutions on the benzene ring of the benzoquinoline moiety are within the scope of the invention. Such replacement of one or more of the hydrogen atoms with deuterium, halogen, C₁-C₁₂ alkanes, ethers, amides, esters, polyols, polyethers, and polyamines, each independently selected, is contemplated. This is a non-limiting list of replacement substituents. Such simple substitutions are also contemplated in other moieties within the compounds.

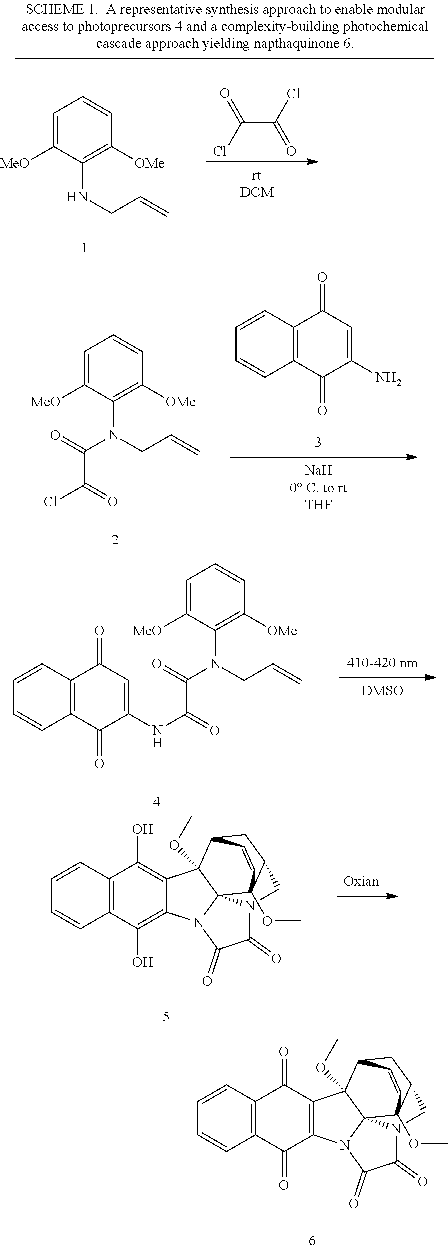

SCHEME 1 illustrates experimentally simple modular access to photoprecursors 4 by linking arylamine (or heteroarylamine) (1) and 2-amino-naphthoquinone (3) with oxalyl amide tether, in accordance with an embodiment. Alternatively, amide formation between aminonaphthoquinone and an aromatic group carrying an appropriately tethered carboxylic acid may be used as the linking mechanism, such as a substituted arylpropanoic acid (or heteroarylpropanoic acid such as furanpropanoic acid). Irradiation of photoprecursors 4 in a solvent such as dimethyl sulfoxide (DMSO) with an ultraviolet (UV) light source, such as light emitting diode (LED) array emitting light in a lamba max (λₘₐₓ) of 410-420 nm wavelength, a mercury UV lamp, solar radiation, among others, triggers their conversion to hydroquinone 5 in a single experimental step photoreaction. Subsequent oxidation of hydroquinone 5 leads to the formation of quinone 6, effectively preventing the cycloreverting of the molecules to their photoprecursors.

TABLE 1
Photocascade products with isolated yields.

| Photoprecursor | Photoproduct(s) | |
|---|---|---|
| 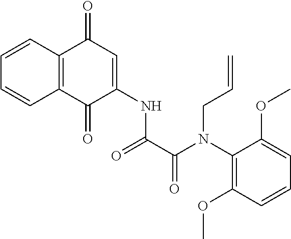 [SMILES: C=CCN(C(=O)C(=O)NC1=CC(=O)c2ccccc2C1=O)c1c(OC)cccc1OC] | 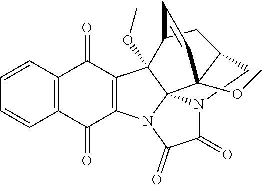 [SMILES: CO[C@@]12C=C[C@H]3C[C@@H]1CN1C(=O)C(=O)N4C5=C(C(=O)c6ccccc6C5=O)[C@]3(OC)[C@]142] | |
| 4a | 6a, 88% | |
| 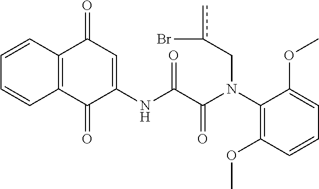 | 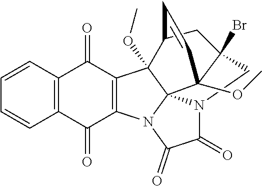 [SMILES: CO[C@@]12C=C[C@H]3C[C@]1(Br)CN1C(=O)C(=O)N4C5=C(C(=O)c6ccccc6C5=O)[C@]3(OC)[C@]142] | |
| 4b | 6b, 84% | |
| 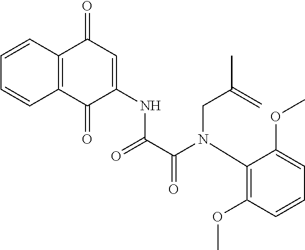 [SMILES: C=C(C)CN(C(=O)C(=O)NC1=CC(=O)c2ccccc2C1=O)c1c(OC)cccc1OC] | 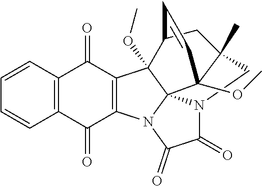 [SMILES: CO[C@@]12C=C[C@H]3C[C@]1(C)CN1C(=O)C(=O)N4C5=C(C(=O)c6ccccc6C5=O)[C@]3(OC)[C@]142] | |
| 4c | 6c, 86% | |
| 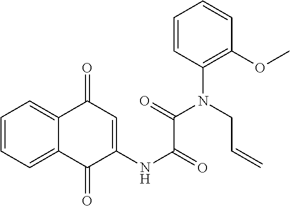 [SMILES: C=CCN(C(=O)C(=O)NC1=CC(=O)c2ccccc2C1=O)c1ccccc1OC] | 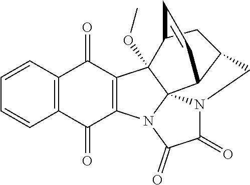 [SMILES: CO[C@]12C3=C(C(=O)c4ccccc4C3=O)N3C(=O)C(=O)N4C[C@H]5C[C@@H]1C=C[C@H]5[C@@]432] | 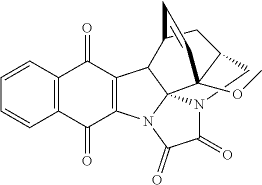 [SMILES: CO[C@@]12C=C[C@H]3C[C@@H]1CN1C(=O)C(=O)N4C5=C(C(=O)c6ccccc6C5=O)C3[C@]142] |
| 4d | major | minor |
| | 60% | 6da, 15% |
| | 6d, 85% (4.1) | |
| 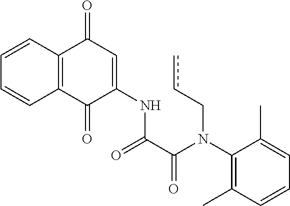 | 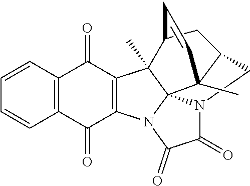 [SMILES: C[C@]12C3=C(C(=O)c4ccccc4C3=O)N3C(=O)C(=O)N4C[C@H]5C[C@@H]1C=C[C@@]5(C)[C@@]432] | |
| 4e | 6e, 76% | |
| 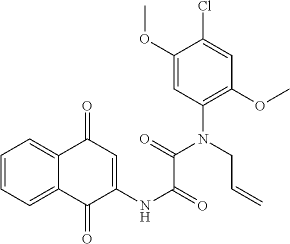 [SMILES: C=CCN(C(=O)C(=O)NC1=CC(=O)c2ccccc2C1=O)c1cc(OC)c(Cl)cc1OC] | 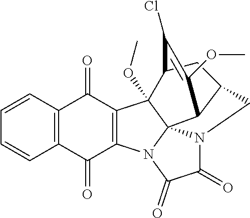 [SMILES: COC1=C(Cl)[C@H]2C[C@@H]3CN4C(=O)C(=O)N5C6=C(C(=O)c7ccccc7C6=O)[C@]2(OC)[C@]45[C@H]13] | 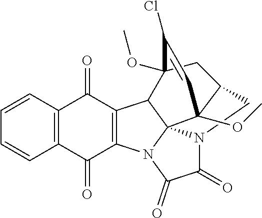 [SMILES: CO[C@]12C[C@@H]3CN4C(=O)C(=O)N5C6=C(C(=O)c7ccccc7C6=O)C1[C@]45[C@]3(OC)C=C2Cl] |
| 4f | 6f, 66% | Not observed |

| | |
|---|---|
| 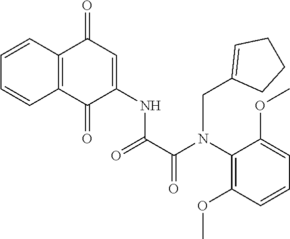 [SMILES: COc1cccc(OC)c1N(CC1=CCCC1)C(=O)C(=O)NC1=CC(=O)c2ccccc2C1=O] | 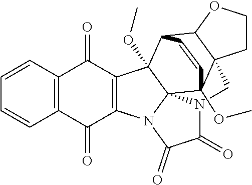 [SMILES: CO[C@@]12C=C[C@H]3C4OCC[C@]41CN1C(=O)C(=O)N4C5=C(C(=O)c6ccccc6C5=O)[C@]3(OC)[C@]142] |
| 4g | 6g, 83% |
| 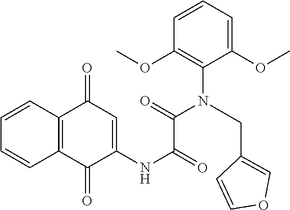 [SMILES: COc1cccc(OC)c1N(Cc1ccoc1)C(=O)C(=O)NC1=CC(=O)c2ccccc2C1=O] | 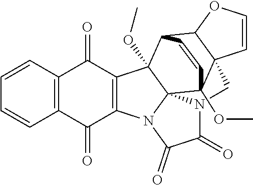 [SMILES: CO[C@@]12C=C[C@H]3C4OC=C[C@]41CN1C(=O)C(=O)N4C5=C(C(=O)c6ccccc6C5=O)[C@]3(OC)[C@]142] |
| 4h | 6h, 84% |
| 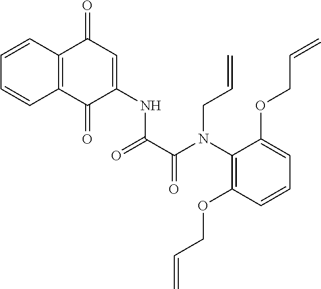 [SMILES: C=CCOc1cccc(OCC=C)c1N(CC=C)C(=O)C(=O)NC1=CC(=O)c2ccccc2C1=O] | 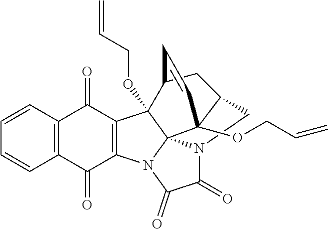 [SMILES: C=CCO[C@@]12C=C[C@H]3C[C@@H]1CN1C(=O)C(=O)N4C5=C(C(=O)c6ccccc6C5=O)[C@]3(OCC=C)[C@]142] |
| 4i | 6i, 75% |
| 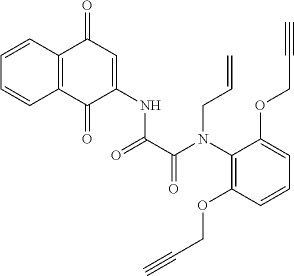 [SMILES: C#CCOc1cccc(OCC#C)c1N(CC=C)C(=O)C(=O)NC1=CC(=O)c2ccccc2C1=O] | 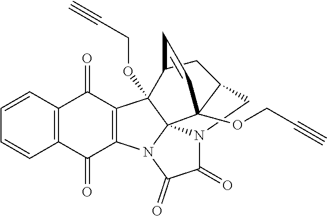 [SMILES: C#CCO[C@@]12C=C[C@H]3C[C@@H]1CN1C(=O)C(=O)N4C5=C(C(=O)c6ccccc6C5=O)[C@]3(OCC#C)[C@]142] |
| 4j | 6j, 86% |
| 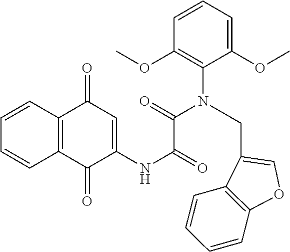 [SMILES: COc1cccc(OC)c1N(Cc1coc2ccccc12)C(=O)C(=O)NC1=CC(=O)c2ccccc2C1=O] | 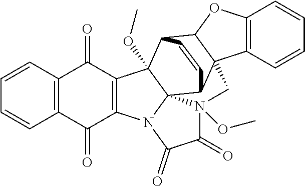 |
| 4k | 6k, 90% |
| 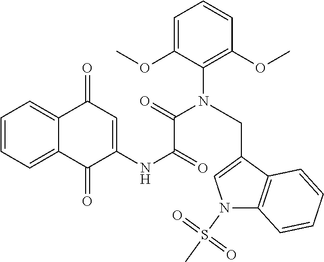 [SMILES: COc1cccc(OC)c1N(Cc1cn(S(C)(=O)=O)c2ccccc12)C(=O)C(=O)NC1=CC(=O)c2ccccc2C1=O] | 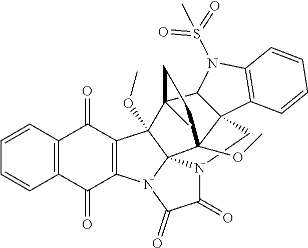 [SMILES: CO[C@@]12CC[C@]3(C)C4N(S(C)(=O)=O)c5ccccc5[C@]41CN1C(=O)C(=O)N4C5=C(C(=O)c6ccccc6C5=O)[C@]3(OC)[C@]142] |
| 4l | 6l, 85% |

The photoreaction mechanism disclosed herein is a [3+2]cycloaddition initiated by Excited State Proton Transfer (ESIPT). During this reaction, a reactive 1,3-diradical is produced. This is in contrast with our earlier work that used a [4+2]cycloaddition via ESIPT-generated 1,4-diradicals.

This photoinduced cascade approach has a broad scope as illustrated by TABLE 1, showing a number of photocascade products with isolated yields, in accordance with certain embodiments. Reaction conditions may be optimized by nuclear magnetic resonance (NMR) scale experiments with acetone, DMSO, methanol, acetonitrile, dichloromethane (DCM), and toluene, for example. In embodiments, DMSO may be particularly suitable as a solvent.

In embodiments, the reactions involved in the synthesis process may be at ambient temperature without explicit temperature control. The structures and stereochemistry of the photoproducts with the aid of DU8ML, a machine learning-augmented density functional theory (DFT) computational method, to perform calculations of the NMR spectra.

For example, in the primary [3+2] photoaddition, the photoprecursor 4d of TABLE 1 displays two possibilities. The major product may subsequently be formed with the active involvement of a methoxy-substituted n-system. A parallel outcome was noted with photoprecursor 4f of TABLE 1, where the minor product was not observable.

Another potentially important feature demonstrated by the photoproducts 6i and 6j is that, due to the ease of outfitting di-allyl or di-propargyl with additional unsaturated functionalities, these products may serve as synthons for subsequent post-photochemical modifications. For instance, ring-closing metathesis and click reactions may be implemented for synthesis of molecules with further complexity.

TABLE 2
Additional photocascade products with isolated yields

| Photoprecursor | Photoproduct(s) | |
|---|---|---|
| 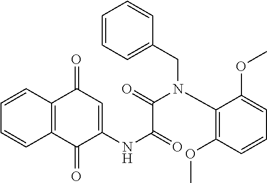 [SMILES: COc1cccc(OC)c1N(Cc1ccccc1)C(=O)C(=O)NC1=CC(=O)c2ccccc2C1=O] | 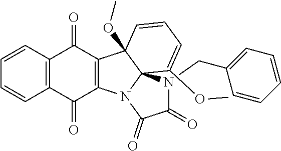 [SMILES: COC1=CC=C[C@@]2(OC)C3=C(C(=O)c4ccccc4C3=O)N3C(=O)C(=O)N(Cc4ccccc4)[C@@]132] | 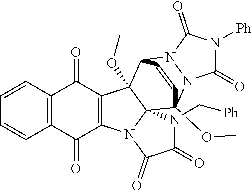 [SMILES: CO[C@@]12C3=C(C(=O)c4ccccc4C3=O)N3C(=O)C(=O)N(Cc4ccccc4)[C@]31[C@]1(OC)C=C[C@H]2n2c(=O)n(-c3ccccc3)c(=O)n21] |
| 4m | 6m, 95% | 10m |
| 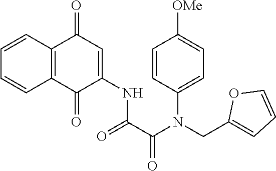 [SMILES: COc1ccc(N(Cc2ccco2)C(=O)C(=O)NC2=CC(=O)c3ccccc3C2=O)cc1] | 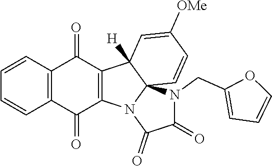 [SMILES: COC1=C[C@H]2C3=C(C(=O)c4ccccc4C3=O)N3C(=O)C(=O)N(Cc4ccco4)[C@]23C=C1] | 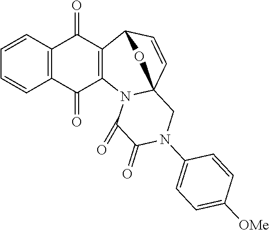 [SMILES: COc1ccc(N2C[C@]34C=C[C@H](O3)C3=C(C(=O)c5ccccc5C3=O)N4C(=O)C2=O)cc1] |
| 4n | 6n, 68% | 7n, 76% |
| | DMSO as a solvent. [3 + 2] | DCM as a solvent, [3 + 4] |
| 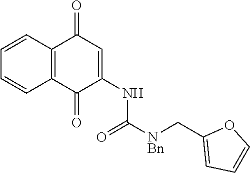 [SMILES: O=C1C=C(NC(=O)N(Cc2ccccc2)Cc2ccco2)C(=O)c2ccccc21] | 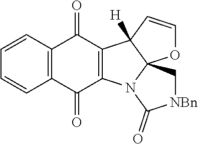 [SMILES: O=C1C2=C(C(=O)c3ccccc31)N1C(=O)N(Cc3ccccc3)C[C@@]13OC=C[C@@H]23] | 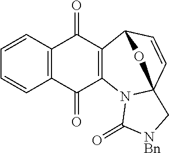 [SMILES: O=C1C2=C(C(=O)c3ccccc31)N1C(=O)N(Cc3ccccc3)C[C@@]13C=C[C@@H]2O3] |
| 4o | 6o, 78% | 7o, 76% |
| | DCM as a solvent [3 + 2] | DMSO as a solvent [3 + 4] |

| | |
|---|---|
| 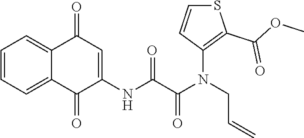 [SMILES: C=CCN(C(=O)C(=O)NC1=CC(=O)c2ccccc2C1=O)c1ccsc1C(=O)OC] | 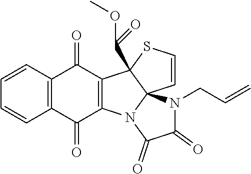 [SMILES: C=CCN1C(=O)C(=O)N2C3=C(C(=O)c4ccccc4C3=O)[C@]3(C(=O)OC)SC=C[C@]123] |
| 4p | 6p, 84% |

This approach may be adapted for polyheterocyclic scaffolds, some examples shown in TABLE 2. Similar to the complex quinone formation summarized in TABLE 1, this pathway may be utilized as a primary photoreaction leading to various quinones embedded in polyheterocyclic scaffolds, even with precursors lacking a reactive alkenyl. These scaffolds, in turn, may serve as synthons in subsequent complexity-building transformations, in certain embodiments.

As an example, photoprecursors 4 were synthesized, equipped with unreactive benzyl, or relatively less reactive 2-furyl-methyl. The N-benzyl photoprecursor 4m yielded a [3+2]cycloaddition product, featuring a reactive diene for further complexity-building transformations. In an embodiment, photoprecursor 4n exhibits solvent-dependent cycloaddition. Irradiation in DMSO may result in the [3+2] photoproduct 6n. Conversely, irradiation in DCM produced [3+4] photoproduct 7m, where the furan n-system was involved in the addition.

Similarly, solvent-dependent cycloaddition may be observed in the case of photoprecursor 4n. For instance, replacing the phenyl n-system with the 3-thiophene n-system results in a heterocyclic fused photoproduct 6p via simple [3+2] addition.

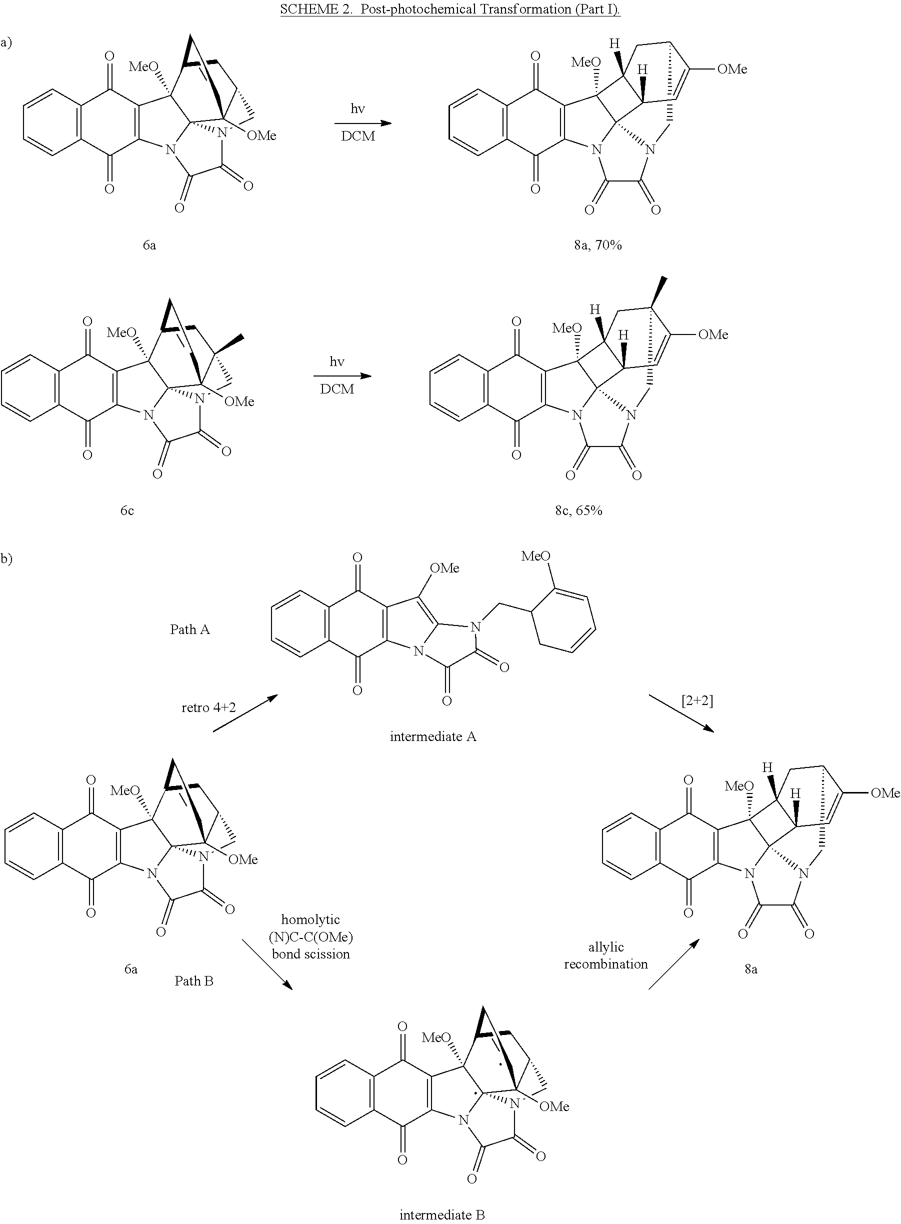

As discussed above, photoprecursor 4 retained its photo-active core after the oxidation of the primary photoproduct 5. Further irradiation of photoproduct 6 may result in further post-photochemical transformation, as illustrated in SCHEME 2. In an embodiment, photoproduct 4a, upon additional irradiation, resulted in a rearranged cyclobutane-fused photoproduct 8a. The homolytic cleavage of the (N)C—C(OMe) bond produces a biradical transition state, which may resonate into its allylic version, in certain embodiments. The recombination of biradicals may lead to the formation of the rearranged product 8a. This photo-rearrangement transformation also may extend to the primary photoproduct 6c, resulting in a rearranged product 8c.

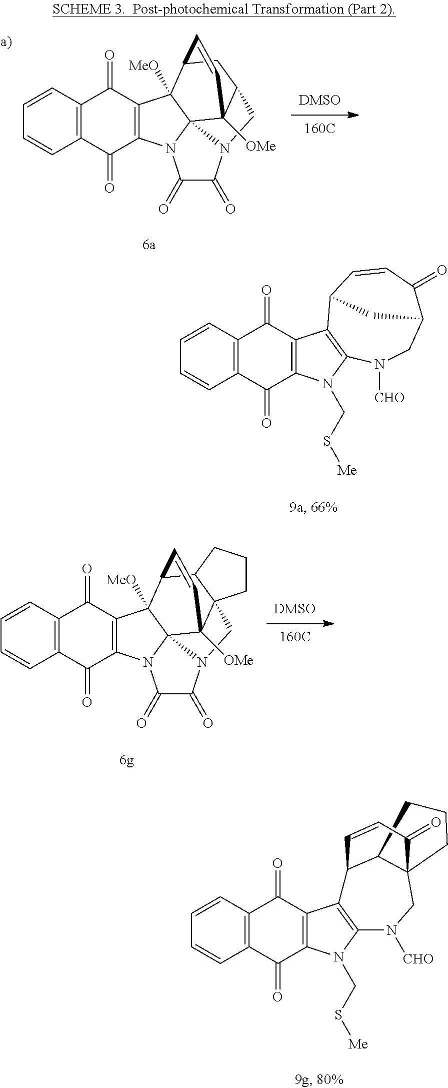

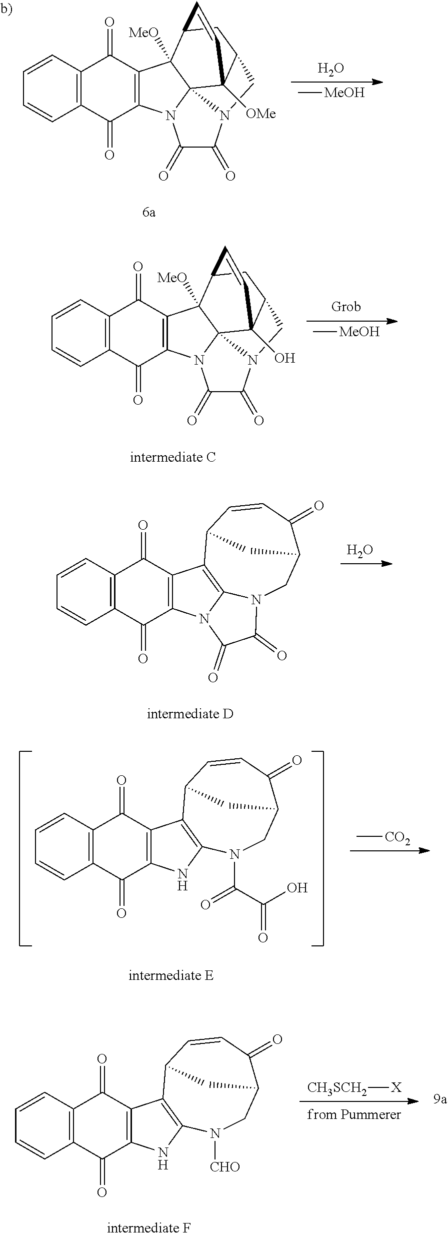

Further, in embodiments, the thermal reaction of photoproduct 6a in DMSO results in the formation of the benzo[f]indole fused product 9a. A mechanistic pathway for this transformation is illustrated in SCHEME 3, in an embodiment. Initially, the demethylation of photoproduct 6a led to an intermediate C This intermediate participated in a Grob-like fragmentation, giving rise to the benzo[f]indole fused azabicyclodecenone D. Subsequently, hydrolysis of the imidazolidinedione moiety, followed by decarboxylation, produces a N-formyl intermediate F. This intermediate may further participate in a Pummerer rearrangement, ultimately yielding the product 9a in certain embodiments.

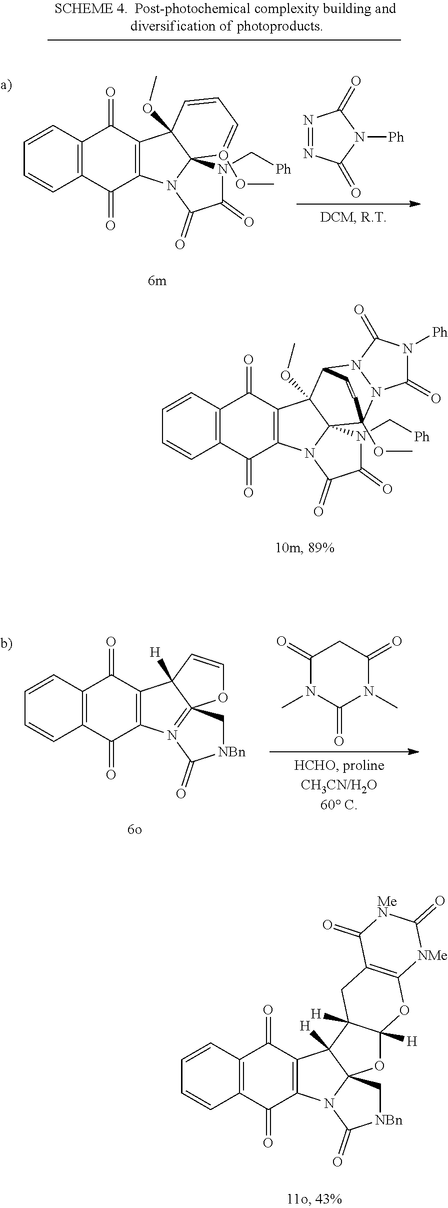

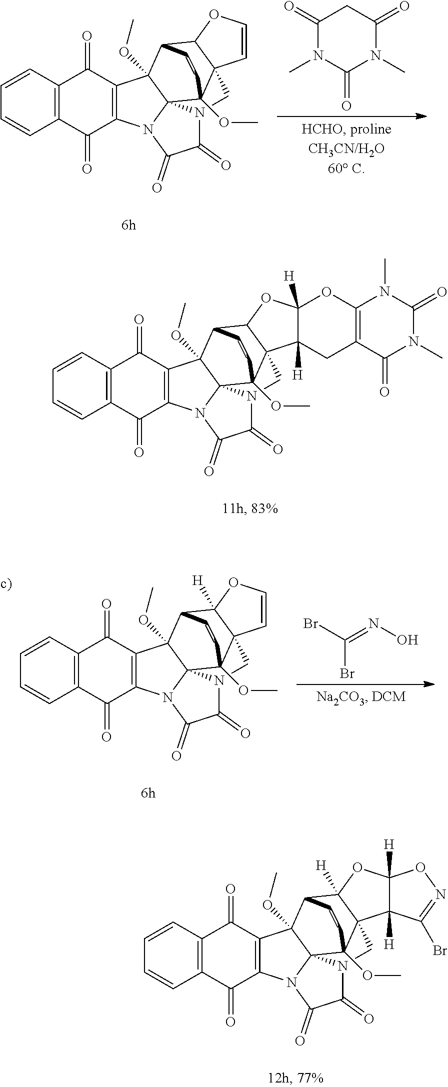

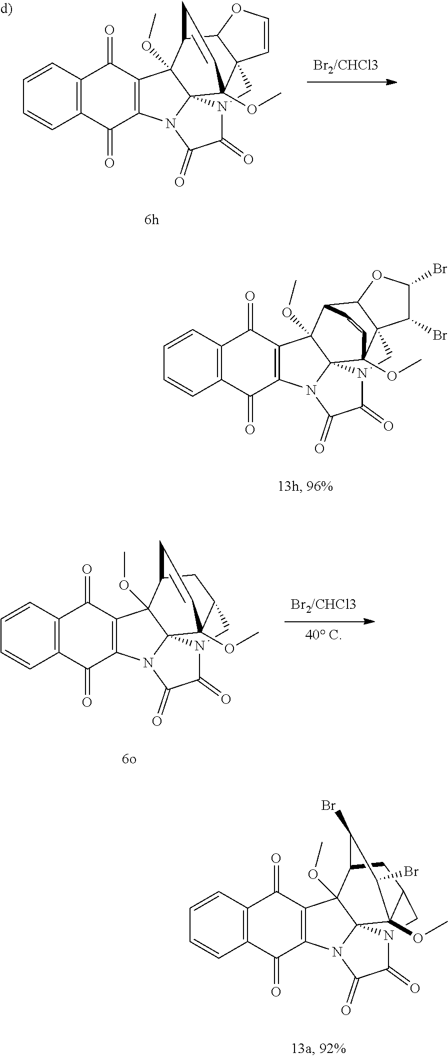

Referring to SCHEME 4, subjected to straightforward yet advantageous post-photochemical transformations, the primary photoproducts may enhance the complexity and diversity of the core scaffold or adorn the cores with various functionalities and (hetero)aromatic pendants. For example, a primary photoproduct 6m contains a reactive cyclohexadiene fragment that can readily participate in [4+2]cycloaddition reactions, including hetero-Diels-Alder reactions with 1,2,4-triazole (a) of SCHEME 4). Subsequently, the dihydrofuran photoproducts (6o and 6h) readily reacted with oxabutadiene, generated in situ from 1,3-dicarbonyl compound, resulted more complexe oxa-Diels-Alder compounds 11o and 11h respectively (5b of Scheme 4).

5c of SCHEME 4 depicts the reaction of 6 h with a 1,3-dipole, bromonitrile oxide, generated in situ from dibromoformaldoxime, leading to the formation of a complex polyheterocycle 12h, in certain embodiments. Given its numerous reactive functionalities, this compound can be believed a valuable synthon for further scaffold diversification.

TABLE 3
Flow reactor photochemical reactions.

| Photoprecursor | Photoproduct(s) |
|---|---|
| 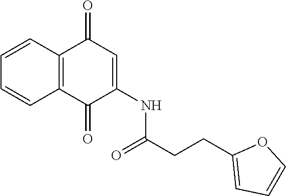 [SMILES: O=C(CCc1ccco1)NC1=CC(=O)c2ccccc2C1=O] | 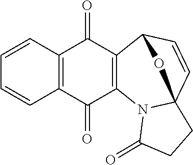 [SMILES: O=C1C2=C(C(=O)c3ccccc31)N1C(=O)CC[C@@]13C=C[C@@H]2O3] |
| 4r | 7r, 86% |
| 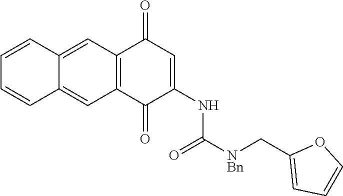 [SMILES: O=C1C=C(NC(=O)N(Cc2ccccc2)Cc2ccco2)C(=O)c2cc3ccccc3cc21] | 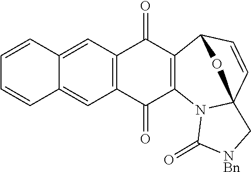 [SMILES: O=C1C2=C(C(=O)c3cc4ccccc4cc31)N1C(=O)N(Cc3ccccc3)C[C@@]13C=C[C@@H]2O3] |
| 4q | 7q, 47% |
| 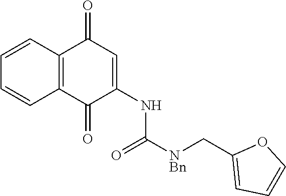 [SMILES: O=C1C=C(NC(=O)N(Cc2ccccc2)Cc2ccco2)C(=O)c2ccccc21] | 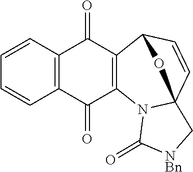 [SMILES: O=C1C2=C(C(=O)c3ccccc31)N1C(=O)N(Cc3ccccc3)C[C@@]13C=C[C@@H]2O3] |
| 4o | 7o, 58% |

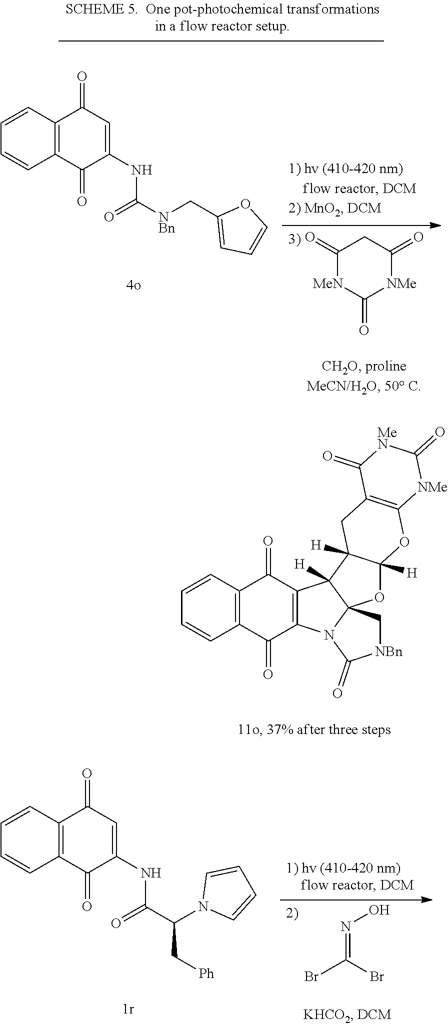

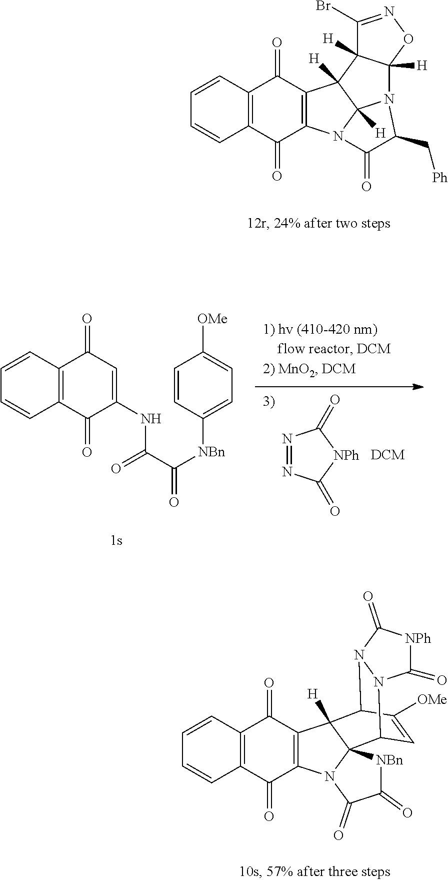

The processes described above may be used for the synthesis of complex molecules particularly suitable for cancer treatment. For example, results of National Cancer Institute (NCI) 5-dose testing of compounds are shown in Table 4.

As generally understood among experts of anticancer research, the one-dose mean graph shows the percent growth of treated cells, in a manner similar to mean graphs from a 5-dose assay (see https://dtp.cancer.gov/discovery_development/nci-60/methodology.htm). The number reported for the one-dose assay is growth relative to a no-drug control, and relative to the time zero number of cells. This approach allows detection of both growth inhibition (values between 0 and 100) and lethality (values less than 0), which is the same approach as the visualization of results for a 5-dose assay. For example, a value of 100 means no growth inhibition. A value of 40 would mean 60% growth inhibition. A value of 0 means no net growth over the course of the experiment. A value of −40 would mean 40% lethality. A value of −100 means all cells are dead.

TABLE 4
5-dose NCI test results
below shows results of NCI five-dose testing. Compounds that showed GI50
(growth inhibition-50%) of at least a 1.5 uM (i.e. micromolar) concentration on various cell
lines are listed, along with the concentration for −50%.

| Compound | Cancer | Cell line | GI50 | TGI | LC50 |
|---|---|---|---|---|---|
| 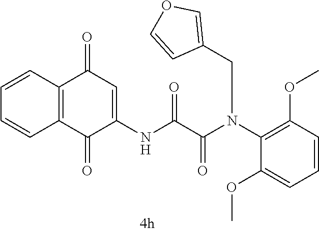 [SMILES: COc1cccc(OC)c1N(Cc1ccoc1)C(=O)C(=O)NC1=CC(=O)c2ccccc2C1=O] | Leukemia | CCRF-CEM HL-60(TB) K-562 MOLT-4 RPMI-8226 | 4.37 × 10⁻⁷ 2.82 × 10⁻⁷ 1.46 × 10⁻⁶ 4.62 × 10⁻⁷ 1.30 × 10⁻⁶ | 1.48 × 10⁻⁶ 5.69 × 10⁻⁷ 2.80 × 10⁻⁶ 1.57 × 10⁻⁶ 2.57 × 10⁻⁶ | 3.89 × 10⁻⁶ 1.48 × 10⁻⁶ 5.37 × 10⁻⁶ 3.98 × 10⁻⁶ 5.12 × 10⁻⁶ |
| | Non-Small Cell | NCI-H522 | 1.33 × 10⁻⁶ | 2.61 × 10⁻⁶ | 5.14 × 10⁻⁶ |
| | Lung Cancer | | | | |
| | Colon Cancer | KM12 | 1.25 × 10⁻⁶ | 3.51 × 10⁻⁶ | 9.84 × 10⁻⁶ |
| | Ovarian Cancer | OVCAR-3 | 1.18 × 10⁻⁶ | 2.42 × 10⁻⁶ | 4.96 × 10⁻⁶ |
| | Breast Cancer | T-47D | 1.48 × 10⁻⁶ | 2.81 × 10⁻⁶ | 5.34 × 10⁻⁶ |
| | | MDA-MB-468 | 2.51 × 10⁻⁷ | 8.99 × 10⁻⁷ | 3.02 × 10⁻⁶ |
| 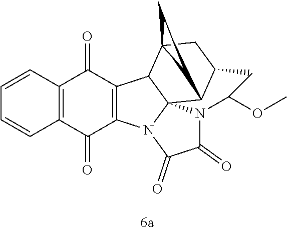 | Ovarian Cancer Breast Cancer | OVCAR-3 MDA-MB-468 | 1.49 × 10⁻⁶ 1.31 × 10⁻⁶ | 4.93 × 10⁻⁶ 3.18 × 10⁻⁶ | 1.96 × 10⁻⁵ 7.73 × 10⁻⁶ |
| 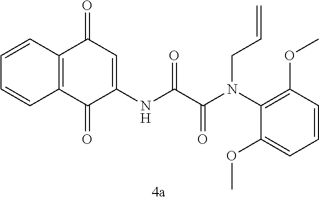 [SMILES: C=CCN(C(=O)C(=O)NC1=CC(=O)c2ccccc2C1=O)c1c(OC)cccc1OC] | Leukemia | CCRF-CEM HL-60(TB) MOLT-4 RPMI-8226 | 7.81 × 10⁻⁷ 2.49 × 10⁻⁷ 4.41 × 10⁻⁷ 1.04 × 10⁻⁶ | 2.04 × 10⁻⁶ 5.42 × 10⁻⁷ 1.62 × 10⁻⁶ 2.22 × 10⁻⁶ | 4.55 × 10⁻⁶ 1.52 × 10⁻⁶ 4.04 × 10⁻⁶ 4.74 × 10⁻⁶ |
| | Non-Small Cell | NCI-H23 | 1.44 × 10⁻⁶ | 2.79 × 10⁻⁶ | 5.39 × 10⁻⁶ |
| | Lung Cancer | NCI-H522 | 1.25 × 10⁻⁶ | 2.51 × 10⁻⁶ | 5.03 × 10⁻⁶ |
| | Colon Cancer | KM12 | 6.26 × 10⁻⁷ | 2.28 × 10⁻⁶ | 6.39 × 10⁻⁶ |
| | CNS Cancer | SNB-75 | 1.43 × 10⁻⁶ | 2.93 × 10⁻⁶ | 5.99 × 10⁻⁶ |
| | Melanoma | UACC-62 | 1.48 × 10⁻⁶ | 2.81 × 10⁻⁶ | 5.35 × 10⁻⁶ |
| | Ovarian Cancer | OVCAR-3 | 1.03 × 10⁻⁶ | 2.21 × 10⁻⁶ | 4.73 × 10⁻⁶ |
| | | OVCAR-4 | 1.22 × 10⁻⁶ | 2.46 × 10⁻⁶ | 4.98 × 10⁻⁶ |
| | | OVCAR-8 | 1.36 × 10⁻⁶ | 2.70 × 10⁻⁶ | 5.36 × 10⁻⁶ |
| | Renal Cancer | ACHN | 1.35 × 10⁻⁶ | 2.66 × 10⁻⁶ | 5.21 × 10⁻⁶ |
| | | UO-31 | 1.44 × 10⁻⁶ | 2.76 × 10⁻⁶ | 5.31 × 10⁻⁶ |
| | Breast Cancer | MCF7 | 1.45 × 10⁻⁶ | 2.78 × 10⁻⁶ | 5.32 × 10⁻⁶ |
| | | T-47D | 1.48 × 10⁻⁶ | 2.81 × 10⁻⁶ | 5.33 × 10⁻⁶ |
| | | MDA-MB-468 | 2.75 × 10⁻⁷ | 8.97 × 10⁻⁷ | 3.00 × 10⁻⁶ |
| 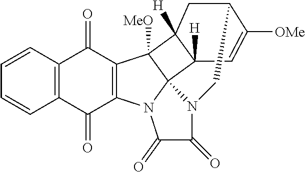 [SMILES: COC1=C[C@H]2[C@H]3C[C@@H]1CN1C(=O)C(=O)N4C5=C(C(=O)c6ccccc6C5=O)[C@]3(OC)[C@]214] | Leukemia | RPMI-8226 SR | 8.03 × 10⁻⁷ 1.40 × 10⁻⁶ | 2.05 × 10⁻⁶ 2.72 × 10⁻⁶ | 4.71 × 10⁻⁶ 5.31 × 10⁻⁶ |
| | Breast Cancer | MCF7 | 9.21 × 10⁻⁷ | 2.94 × 10⁻⁶ | 8.87 × 10⁻⁶ |
| 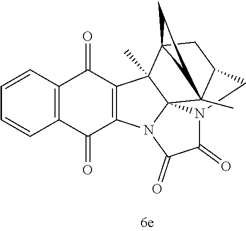 | Leukemia | CCRF-CEM HL-60(TB) | 1.23 × 10⁻⁶ 2.67 × 10⁻⁷ | 2.48 × 10⁻⁶ 1.06 × 10⁻⁶ | 5.00 × 10⁻⁶ 3.28 × 10⁻⁶ |
| | Melanoma | SK-MEL-2 | 1.27 × 10⁻⁶ | 2.57 × 10⁻⁶ | 5.20 × 10⁻⁶ |
| | | SK-MEL-5 | 1.30 × 10⁻⁶ | 3.08 × 10⁻⁶ | 7.32 × 10⁻⁶ |
| | Ovarian Cancer | OVCAR-4 | 1.36 × 10⁻⁶ | 2.65 × 10⁻⁶ | 5.17 × 10⁻⁶ |
| | Breast Cancer | MDA-MB-468 | 3.84 × 10⁻⁷ | 1.55 × 10⁻⁶ | 4.03 × 10⁻⁶ |
| 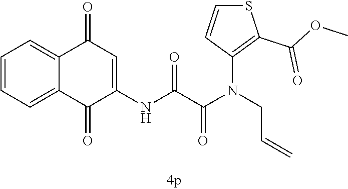 [SMILES: C=CCN(C(=O)C(=O)NC1=CC(=O)c2ccccc2C1=O)c1ccsc1C(=O)OC] | Leukemia | CCRF-CEM HL-60(TB) K-562 MOLT-4 RPMI-8226 | 1.30 × 10⁻⁶ 1.08 × 10⁻⁶ 1.45 × 10⁻⁶ 1.11 × 10⁻⁶ 1.47 × 10⁻⁶ | 2.57 × 10⁻⁶ 2.27 × 10⁻⁶ 2.77 × 10⁻⁶ 2.31 × 10⁻⁶ 2.79 × 10⁻⁶ | 5.07 × 10⁻⁶ 4.79 × 10⁻⁶ 5.28 × 10⁻⁶ 4.81 × 10⁻⁶ 5.28 × 10⁻⁶ |
| | Non-Small Cell | NCI-H522 | 1.45 × 10⁻⁶ | 2.77 × 10⁻⁶ | 5.28 × 10⁻⁶ |
| | Lung Cancer | | | | |
| | Breast Cancer | T-47D | 1.32 × 10⁻⁶ | 2.61 × 10⁻⁶ | 5.14 × 10⁻⁶ |
| | | MDA-MB-468 | 2.26 × 10⁻⁷ | 9.41 × 10⁻⁷ | 3.09 × 10⁻⁶ |
| 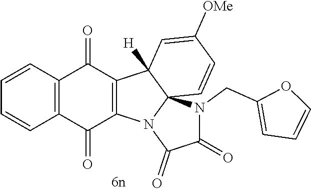 [SMILES: COC1=C[C@H]2C3=C(C(=O)c4ccccc4C3=O)N3C(=O)C(=O)N(Cc4ccco4)[C@]23C=C1] | Melanoma Ovarian Cancer Breast Cancer | SK-MEL-5 IGROV1 MDA-MB-468 | 1.13 × 10⁻⁶ 1.38 × 10⁻⁶ 1.77 × 10⁻⁷ | 3.21 × 10⁻⁶ 6.38 × 10⁻⁶ 1.35 × 10⁻⁶ | 9.08 × 10⁻⁶ 1.00 × 10⁻⁴ 7.96 × 10⁻⁶ |
| 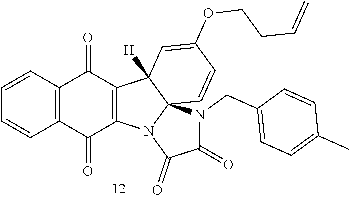 [SMILES: C=CCCOC1=C[C@H]2C3=C(C(=O)c4ccccc4C3=O)N3C(=O)C(=O)N(Cc4ccc(C)cc4)[C@]23C=C1] | Leukemia Ovarian Cancer Prostate Cancer | SR OVCAR-4 MDA-MB-468 | 8.62 × 10⁻⁷ 8.75 × 10⁻⁷ 2.09 × 10⁻⁸ | 1.00 × 10⁻⁴ 7.04 × 10⁻⁶ 6.86 × 10⁻⁷ | 1.00 × 10⁻⁴ 1.00 × 10⁻⁴ 1.00 × 10⁻⁴ |
| 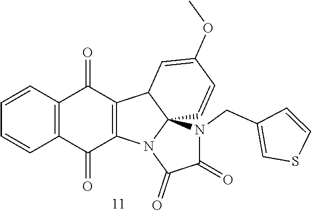 | Non-Small Cell Lung Cancer | EKVX | 1.11 × 10⁻⁶ | 5.75 × 10⁻⁶ | 2.57 × 10⁻⁵ |
| | Melanoma | SK-MEL-5 | 1.00 × 10⁻⁸ | 2.16 × 10⁻⁶ | 6.04 × 10⁻⁶ |
| | Ovarian Cancer | IGROV1 | 1.27 × 10⁻⁶ | 1.40 × 10⁻⁵ | 7.05 × 10⁻⁵ |
| | Renal Cancer | RXF 393 | 1.43 × 10⁻⁶ | 3.76 × 10⁻⁶ | 9.92 × 10⁻⁶ |
| | Breast Cancer | MCF7 | 1.26 × 10⁻⁶ | 5.88 × 10⁻⁶ | 2.52 × 10⁻⁵ |
| | | MDA-MB-468 | 1.13 × 10⁻⁷ | 8.16 × 10⁻⁷ | 9.32 × 10⁻⁶ |
| 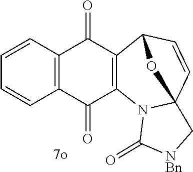 [SMILES: O=C1C2=C(C(=O)c3ccccc31)N1C(=O)N(Cc3ccccc3)C[C@@]13C=C[C@@H]2O3] | Breast Cancer | T-47D MDA-MB-468 | 1.39 × 10⁻⁶ 4.72 × 10⁻⁷ | 3.26 × 10⁻⁶ 1.59 × 10⁻⁶ | 7.64 × 10⁻⁶ 4.08 × 10⁻⁶ |
| 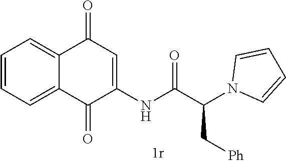 [SMILES: O=C1C=C(NC(=O)[C@H](Cc2ccccc2)n2cccc2)C(=O)c2ccccc21] | Leukemia | CCRF-CEM MOLT-4 | 1.11 × 10⁻⁶ 6.85 × 10⁻⁷ | 2.32 × 10⁻⁶ 1.84 × 10⁻⁶ | 4.84 × 10⁻⁶ 4.29 × 10⁻⁶ |
| | Breast Cancer | T-47D | 1.42 × 10⁻⁶ | 2.74 × 10⁻⁶ | 5.26 × 10⁻⁶ |

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms-even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding”-whether explicitly discussed or not-and, conversely, were there only disclosure of the act of “protruding”, such a disclosure should be understood to encompass disclosure of a “protrusion”. Such changes and alternative terms are to be understood to be explicitly included in the description.

### EXAMPLES

Experimental procedures for compounds synthesized are shown below:

Common solvents were purchased from Fisher Scientific and used as is. Common reagents, aldehydes, and alkyl halides were purchased from Sigma-Aldrich, TCI America, AK Scientific, Oakwood Chemical or AstaTech and used without additional purification. NMR spectra were recorded at 25° C. on a Bruker Avance III 500 MHz instrument in CDCl₃ (unless noted otherwise) using residual solvent peaks as an internal standard (δ_{H} 7.26 ppm, δ_{C} 77.16 ppm for CDCl₃; δ_{H} 2.50 ppm, δ_{C} 39.52 ppm for DMSO-d₆). The description of signals includes s=singlet, d=doublet, dd=doublet of doublets, t=triplet, dt=doublet of triplets, td=triplet of doublets, q=quartet, m=multiplet, br.s=broad singlet. The structural assignments were made with additional information from gCOSY, gHSQC, and gHMBC experiments. X-ray structures were obtained with a Bruker APEX II instrument. High-resolution mass spectra were obtained on a Waters Synapt G2 HDMS Quadrupole/ToF mass spectrometer with electrospray ionization (Central Analytical Laboratory, University of Colorado Boulder). Flash column chromatography was performed using Teledyne Ultra-Pure Silica Gel (230-400 mesh) on a Teledyne Isco Combiflash Rf. The light-promoted reactions were carried out using An irradiator containing two visible light LEDs (High Power LED Star #A008-UV410-48; λₘₐₓ=410-420 nm; board configuration: 3-Up A008) installed on a heat sink with fan and driven by a RACD 30-700 power supply (output: 700 mA, 10-43 VDC), was placed ca. 2-5 mm away from the irradiation window. Borosilicate glass reaction vessels were typically distanced from a light source in a 5-7 cm range.

### Precursors

### General Procedure a for the Preparation of 2-amino-1,4-naphthalenedione (3)

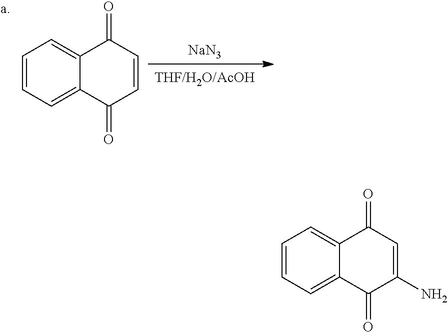 [SMILES: O=C1C=CC(=O)c2ccccc21 ; NC1=CC(=O)c2ccccc2C1=O ; "NaN3"]

Prepared according to a modified literature protocol.^{1,2} 1,4-Naphthoquinone (6.39 mmol) was suspended in THF-water 4:1 (20 mL). Solution of sodium azide (19.2 mmol) in water-AcOH 3:1 (4 mL) was added, and the mixture was stirred at ambient temperature for 12 h. After the removal of volatiles by evaporation, water (30 mL) was added, and then the product was extracted with DCM (4×150 mL). The combined organic layers were washed with sat. aq. NaHCO₃ (30 mL), dried over anhydrous Na₂SO₄, filtered, and the solvent was removed under reduced pressure to afford a title compound (3). ¹H NMR (500 MHz, CDCl₃) δ 8.07 (ddd, J=10.8, 7.7, 0.9 Hz, 2H), 7.73 (td, J=7.6, 1.3 Hz, 1H), 7.64 (td, J=7.6, 1.3 Hz, 1H), 6.00 (s, 1H), 5.13 (br s, 2H).

### General Procedure B for the Preparation of N-Alkylation Reactions

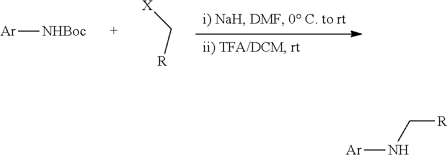

To a stirred solution of Boc-protected aniline (1.0 mmol) in DMF (10 mL) were sequentially added NaH (60 mg, 1.5 mmol, 60% in mineral oil) and alkyl bromide/chloride (1.25 mmol) at 0° C. The resulting solution was stirred at ambient temperature for 15 h. After the completion of the reaction (the progress of the reaction was monitored by ¹H NMR), water (20 mL) was added at 0° C., and organics were extracted with ethyl acetate (3×30 mL). The combined organic extracts were washed with water (2×50 mL), and dried over anhydrous Na₂SO₄, then concentrated under reduced pressure to give a crude yellow oil. The crude product was dissolved in DCM and TFA (10 mL, 5:1 v/v ratio), and stirred at ambient temperature for 3 h. The progress of the reaction was monitored by ¹H NMR. After completion of the reaction, the volatiles were removed under vacuum, and a saturated solution of NaHCO₃ (10 mL) was added. Organics were extracted with DCM (3×30 mL), and the combined organic extracts were dried over anhydrous Na₂SO₄, and the solvent was removed under reduced pressure to afford secondary anilines. The crude compound was purified by flash chromatography on silica gel to give the desired products.

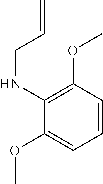

N-AIlyl-2,6-dimethoxyaniline (1a): General procedure B was followed on 0.915 g (3.61 mmol) scale of tert-butyl (2,6-dimethoxyphenyl)carbamate. The crude product was purified by flash chromatography (SiO₂, 0-15% of ethyl acetate in hexanes) which afforded 0.58 g (83%) of the title compound (1a) as a light brown liquid. ¹H NMR (500 MHz, CDCl₃) δ 6.82 (t, J=8.2 Hz, 1H), 6.55 (d, J=8.3 Hz, 2H), 5.94 (ddt, J=16.5, 10.2, 5.9 Hz, 1H), 5.17 (dq, J=17.1, 1.8 Hz, 1H), 5.04 (dq, J=10.2, 1.5 Hz, 1H), 3.88-3.76 (m, 8H); ¹³_{C}{¹H}NMR (126 MHz, CDCl₃) δ 151.20, 137.51, 127.14, 120.16, 115.25, 104.82, 56.03, 49.81. HRMS (ESI) m/z: [M+H]⁺ Calcd for C₁₁H₁₆NO₂ 194.1181; found 194.1187.

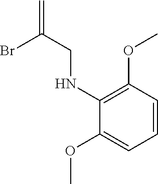

N-(2-Bromoallyl)-2,6-dimethoxyaniline (1b): General procedure B was followed on 0.680 g (2.68 mmol) scale of tert-butyl (2,6-dimethoxyphenyl)carbamate. The crude product was purified by flash chromatography (SiO₂, 0-15% of ethyl acetate in hexanes) which afforded 0.68 g (93%) of the title compound (1b) as a light brown liquid. ¹H NMR (500 MHz, CDCl₃) δ 6.85 (t, J=8.3 Hz, 1H), 6.55 (d, J=8.4 Hz, 2H), 5.74-5.69 (m, 1H), 5.43 (d, J=2.0 Hz, 1H), 4.72 (s, 1H), 4.20-4.16 (m, 2H), 3.85 (s, 6H); ¹³C {¹H}NMR (126 MHz, CDCl₃) δ 150.95, 132.55, 124.52, 121.00, 117.25, 104.97, 56.19, 54.88. HRMS (ESI) m/z: [M+H]⁺ calcd for C₁₁H₁₅BrNO₂ 272.0286; found 272.0294.

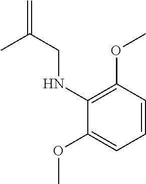

2,6-Dimethoxy-N-(2-methylallyl)aniline (1c): General procedure B was followed on 0.915 g (3.61 mmol) scale of tert-butyl (2,6-dimethoxyphenyl)carbamate. The crude product was purified by flash chromatography (SiO₂, 0-15% of ethyl acetate in hexanes) which afforded 0.58 g (77%) of the title compound (1c) as a light brown liquid. ¹H NMR (500 MHz, CDCl₃) δ 6.79 (t, J=8.3 Hz, 1H), 6.54 (d, J=8.2 Hz, 2H), 4.89-4.85 (m, 1H), 4.79-4.75 (m, 1H), 4.11 (s, 1H), 3.85-3.80 (m, 8H), 1.74 (s, 3H); ¹³C {¹H}NMR (126 MHz, CDCl₃) δ 150.88, 144.84, 127.35, 119.75, 110.48, 104.93, 56.04, 52.90, 20.74. HRMS (ESI) m/z: [M+H]⁺ calcd for C₁₂H₁₈NO₂ 208.1338; found 208.1342.

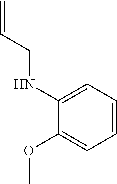

N-Allyl-2-methoxyaniline (1d): General procedure B was followed on 1.0 g (4.48 mmol) scale of tert-butyl (2-methoxyphenyl)carbamate The crude product was purified by flash chromatography (SiO₂, 0-15% of ethyl acetate in hexanes) which afforded 0.72 g (98%) of the title compound (1d) as a light brown liquid. ¹H NMR (500 MHz, CDCl₃) δ 6.89 (td, J=7.6, 1.4 Hz, 1H), 6.80 (dd, J=7.9, 1.4 Hz, 1H), 6.70 (td, J=7.7, 1.6 Hz, 1H), 6.64 (dd, J=7.8, 1.6 Hz, 1H), 6.01 (ddt, J=17.2, 10.5, 5.4 Hz, 1H), 5.32 (dq, J=17.1, 1.7 Hz, 1H), 5.19 (dq, J=10.3, 1.5 Hz, 1H), 4.40 (s, 1H), 3.88 (s, 3H), 3.83 (dt, J=5.3, 1.7 Hz, 2H); ¹³C {¹H}NMR (126 MHz, CDCl₃) δ 147.01, 138.17, 135.74, 121.38, 116.72, 116.17, 110.31, 109.59, 55.57, 46.44. HRMS (ESI) m/z: [M+H]⁺ calcd for C₁₀H₁₄NO 164.1075; found 164.1076.

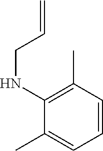

N-Allyl-2,6-dimethylaniline (1e): General procedure B was followed on 1.5 g (6.78 mmol) scale of tert-butyl (2,6-dimethylphenyl)carbamate. The crude product was purified by flash chromatography (SiO₂, 0-15% of ethyl acetate in hexanes) which afforded 0.95 g (87%) of the title compound (1e) as a light brown liquid. ¹H NMR (500 MHz, CDCl₃) δ 7.00 (d, J=7.5 Hz, 2H), 6.83 (t, J=7.5 Hz, 1H), 5.99 (ddt, J=16.4, 10.1, 6.0 Hz, 1H), 5.28 (dq, J=17.1, 1.7 Hz, 1H), 5.12 (dq, J=10.2, 1.4 Hz, 1H), 3.60 (dt, J=6.1, 1.4 Hz, 2H), 2.96 (s, 1H), 2.30 (s, 6H); ¹³C {¹H}NMR (126 MHz, CDCl₃) δ 146.01, 136.87, 129.64, 128.93, 122.10, 116.08, 51.36, 18.65. HRMS (ESI) m/z: [M+H]⁺ calcd for C₁₁H₁₆N 162.1283; found 162.1289.

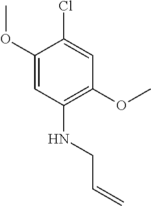

N-Allyl-4-chloro-2,5-dimethoxyaniline (1f): General procedure B was followed on 1.2 g (4.17 mmol) scale of 1,1-dimethylethyl N-(4-chloro-2,5-dimethoxyphenyl)carbamate. The crude product was purified by flash chromatography (SiO₂, 0-15% of ethyl acetate in hexanes) which afforded 0.817 g (86%) of the title compound (1f) as a light brown liquid. ¹H NMR (500 MHz, CDCl₃) δ 6.75 (s, 1H), 6.26 (s, 1H), 5.95 (ddt, J=17.3, 10.5, 5.3 Hz, 1H), 5.29 (dq, J=17.2, 1.8 Hz, 1H), 5.19 (dq, J=10.2, 1.6 Hz, 1H), 4.39 (s, 1H), 3.85-3.77 (m, 8H). ¹³C {¹H}NMR (126 MHz, CDCl₃) δ 149.91, 141.30, 137.82, 135.23, 116.53, 111.97, 108.26, 97.10, 57.10, 56.31, 46.47. HRMS (ESI) m/z: [M+H]⁺ calcd for C₁₁H₁₅ClNO₂ 228.0791; found 228.0807.

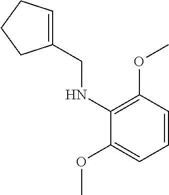 [SMILES: COc1cccc(OC)c1NCC1=CCCC1]

N-(Cyclopent-1-en-1-ylmethyl)-2,6-dimethoxyaniline (1g): General procedure B was followed on 1.56 g (6.16 mmol) scale of tert-butyl (2,6-dimethoxyphenyl)carbamate. The crude product was purified by flash chromatography (SiO₂, 0-15% of ethyl acetate in hexanes) which afforded 0.5 g (35%) of the title compound (1g) as a light brown liquid. ¹H NMR (500 MHz, CDCl3) δ 6.80 (t, J=8.2 Hz, 1H), 6.54 (d, J=8.3 Hz, 2H), 5.53 (dd, J=3.6, 1.8 Hz, 1H), 3.91-3.87 (m, 2H), 3.86-3.81 (m, 7H), 2.41-2.17 (m, 4H), 1.85 (p, J=7.5 Hz, 2H); 13C {¹H}NMR (126 MHz, CDCl3) δ 151.04, 144.02, 127.78, 124.48, 119.84, 104.88, 56.03, 47.65, 33.80, 32.48, 23.58. HRMS (ESI) m/z: [M+H]+ calcd for C₁₄H₂₀NO₂ 234.1494; found 234.1518.

### General Procedure C for the Preparation of Secondary Amines Via Reductive Amination

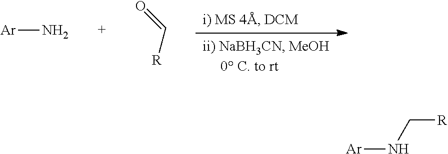

Primary aniline (4.2 mmol) was dissolved in anhydrous DCM (20 mL). Aldehyde (4.3 mmol) and molecular sieves 4 Å were added, and the mixture was stirred at ambient temperature till completion (monitored by ¹H NMR). Solids were filtered off and washed with DCM. The filtrate was concentrated in vacuo. The residue was dissolved in absolute methanol (20 mL), sodium cyanoborohydride (4.6 mmol) was added at 0° C., and the mixture was stirred overnight. Upon completion (as indicated by TLC), methanol was evaporated, the residue was partitioned between DCM (20 mL) and water (15 mL). Organic layer was separated, washed with water (2×15 mL) and brine (10 mL), dried over anhydrous Na₂SO₄, and the solvent was removed under reduced pressure to afford secondary anilines. The majority of products were used for the next steps without chromatographical purification.

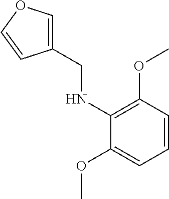 [SMILES: COc1cccc(OC)c1NCc1ccoc1]

N-(Furan-3-ylmethyl)-2,6-dimethoxyaniline (1h): General procedure C was followed on 1.6 g (10.44 mmol) scale of 2,6-dimethoxyaniline. The crude product was purified by flash chromatography (SiO₂, 0-15% of ethyl acetate in hexanes) which afforded 1.7 g (70%) of the title compound (1h) as a light brown liquid. ¹H NMR (500 MHz, CDCl₃) δ 7.35-7.29 (m, 2H), 6.82 (t, J=8.3 Hz, 1H), 6.57-6.52 (m, 2H), 6.34 (t, J=1.2 Hz, 1H), 4.27 (s, 2H), 3.83 (s, 6H); ¹³C {¹H}NMR (126 MHz, CDCl₃) δ 151.24, 142.93, 139.68, 127.15, 124.92, 120.37, 110.49, 104.86, 56.05, 41.98. HRMS (ESI) m/z: [M+H]⁺ calcd for C₁₃H₁₆NO₃ 234.1130; found 234.1117.

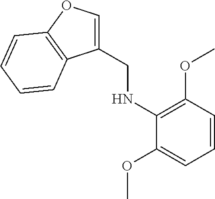 [SMILES: COc1cccc(OC)c1NCc1coc2ccccc12]

N-(Benzofuran-3-ylmethyl)-2,6-dimethoxyaniline (1i): General procedure C was followed on 1.05 g (6.85 mmol) scale of 2,6-dimethoxyaniline. The crude product was purified by flash chromatography (SiO₂, 0-15% of ethyl acetate in hexanes) which afforded 1.46 g (75%) of the title compound (ii) as a light brown liquid. ¹H NMR (500 MHz, CDCl₃) δ 7.67-7.62 (m, 1H), 7.55 (s, 1H), 7.50-7.45 (m, 1H), 7.34-7.18 (m, 2H), 6.90-6.80 (m, 1H), 6.61-6.53 (m, 2H), 4.56 (s, 2H), 3.85-3.81 (m, 7H); ¹³C {¹H}NMR (126 MHz, CDCl₃) δ 155.59, 151.38, 142.24, 127.65, 127.20, 124.31, 122.47, 120.62, 120.17, 120.10, 111.50, 104.93, 56.04, 41.16. HRMS (ESI) m/z: [M+H]⁺ calcd for C₁₇H₁₈NO₃ 284.1287; found 284.1323.

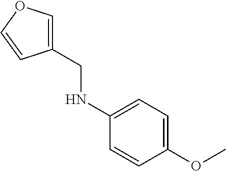

N-(Furan-3-ylmethyl)-4-methoxyaniline (1j): General procedure C was followed on 1.6 g (10.44 mmol) scale of 2,6-dimethoxyaniline. The crude product was purified by flash chromatography (SiO₂, 0-15% of ethyl acetate in hexanes) which afforded 1.8 g (85%) of the title compound (1j) as a light brown liquid. ¹H NMR (500 MHz, CDCl₃) δ 7.36 (d, J=1.9, 0.8 Hz, 1H), 6.82-6.75 (m, 2H), 6.69-6.62 (m, 2H), 6.32 (dd, J=3.2, 1.9 Hz, 1H), 6.24-6.20 (m, 1H), 4.28 (s, 2H), 3.85 (s, 1H), 3.75 (s, 3H); ¹³C {¹H}NMR (126 MHz, CDCl₃) δ 153.12, 152.77, 142.02, 141.86, 114.99, 114.85, 110.44, 107.09, 55.90, 42.62; HRMS (ESI) m/z: [M+H]⁺ calcd for C₁₂H₁₃NO₂ 203.0941, found 203.0947.

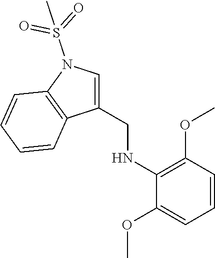 [SMILES: COc1cccc(OC)c1NCc1cn(S(C)(=O)=O)c2ccccc12]

2,6-Dimethoxy-N-((1-(methylsulfonyl)-1H-indol-3-yl)methyl)aniline (6f):^{s2} Following the general procedure d, compound 1m (0.31 g, 54%) was obtained from 1-(methylsulfonyl)-1H-indole-3-carbaldehyde (250 mg; 1.64 mmol). ¹H NMR (500 MHz, CDCl₃) δ 7.91 (dt, J=8.2, 1.0 Hz, 1H), 7.73 (dt, J=7.5, 1.0 Hz, 1H), 7.41-7.31 (m, 3H), 6.86 (t, J=8.3 Hz, 1H), 6.56 (d, J=8.3 Hz, 2H), 4.58 (d, J=1.1 Hz, 2H), 3.82 (s, 6H), 2.94 (s, 3H); ¹³C {¹H}NMR (126 MHz, CDCl₃) δ 151.36 (2_{C}), 135.60, 130.34, 126.63, 124.94, 123.70, 123.36, 122.26, 120.75, 120.12, 113.26, 104.69, 55.90, 42.04, 40.11. HRMS (ESI) m/z: [M+H]+ Calcd for C₁₈H₂₀N₂O₄S 361.1217; found 361.1274.

### General Procedure D for the Preparation of N-Allyl-2,6-Dialkylated-Aniline

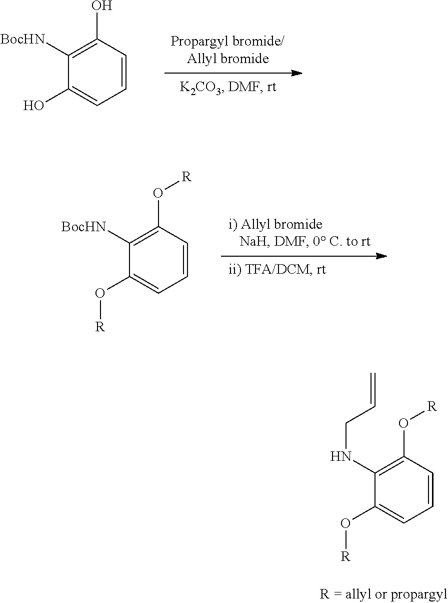

To a stirred solution of tert-butyl (2,6-dihydroxyphenyl)carbamate (1 eq) and K₂CO₃ (3 eq) in DMF (1 mL/mmol), propargyl bromide or allyl bromide (2.3 eq) was added. The reaction was left with stirring overnight at rt. The mixture was filtered and added water to the filtrate. The aqueous phase was extracted with Et₂O twice. The combined organic phase was washed with brine and dried over Na₂SO₄. After removal of the solvent under vacuum, crude oil was purified by flash chromatography on silica gel to give the desired N-protected, 2,6-dialkylated-aniline product. For the next step general procedure A was followed for preparation of N-allyl-2,6-dialkylated-aniline.

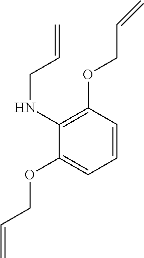 [SMILES: C=CCNc1c(OCC=C)cccc1OCC=C]

N-Allyl-2,6-bis(allyloxy)aniline (1k): General procedure D was followed on 0.676 g (3.0 mmol) scale of tert-butyl (2,6-dihydroxyphenyl)carbamate. The crude product was purified by flash chromatography (SiO₂, 0-15% of ethyl acetate in hexanes) which afforded 0.4 g (54%) of the title compound (1k) as a light brown liquid. ¹H NMR (500 MHz, CDCl₃) δ 6.75 (t, J=8.2 Hz, 1H), 6.54 (d, J=8.2 Hz, 2H), 6.13-6.02 (m, 2H), 6.00-5.89 (m, 1H), 5.44-5.36 (m, 2H), 5.30-5.23 (m, 2H), 5.23-5.14 (m, 1H), 5.04 (dd, J=10.3, 1.6 Hz, 1H), 4.60-4.53 (m, 4H), 4.06 (s, 1H), 3.95-3.88 (m, 2H); ¹³C {¹H}NMR (126 MHz, CDCl₃) δ 150.04, 137.56, 133.75, 128.08, 119.72, 117.41, 115.25, 106.70, 69.84, 49.68. HRMS (ESI) m/z: [M+H]⁺ calcd for C₁₅H₂₀NO₂ 246.1494; found 246.1495.

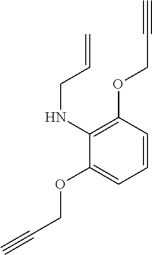 [SMILES: C#CCOc1cccc(OCC#C)c1NCC=C]

N-Allyl-2,6-bis(prop-2-yn-1-yloxy)aniline (1l): General procedure D was followed on 2.07 g (9.19 mmol) of tert-butyl (2,6-dihydroxyphenyl)carbamate. The crude product was purified by flash chromatography (SiO₂, 0-25% of ethyl acetate in hexanes) which afforded 1.3 g (59%) of the title compound (1l) as a light brown liquid. ¹H NMR (500 MHz, CDCl₃) δ 6.83-6.76 (m, 1H), 6.72-6.67 (m, 2H), 5.94 (ddt, J=16.3, 10.3, 5.9 Hz, 1H), 5.20 (dq, J=17.0, 1.6 Hz, 1H), 5.05 (dt, J=10.5, 1.5 Hz, 1H), 4.71 (d, J=2.3 Hz, 4H), 3.93-3.88 (m, 2H), 2.50 (t, J=2.4 Hz, 2H); ¹³C {¹H}NMR (126 MHz, CDCl₃) δ 149.10, 137.25, 128.95, 119.59, 115.57, 108.09, 78.94, 75.56, 56.91, 49.72. HRMS (ESI) m/z: [M+Na]⁺ calcd for C₁₅H₁₅NO₂Na 264.1000; found 264.0997.

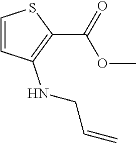

Methyl 3-(allylamino)thiophene-2-carboxylate (10): To a stirred solution of methyl 3-aminothiophene-2-carboxylate (0.5 g, 3.1 mmol) in acetone (15 mL) was added allyl bromide (0.44 mL, 4.7 mmol), and K₂CO₃ (01.2 g, 9.3 mmol). The mixture was refluxed for 16 h. Observed the formation of mono alkylated product, bis alkylated product and unreacted methyl 3-aminothiophene-2-carboxylate (5:3:2). Acetone was evaporated, the residue was partitioned between ethyl acetate (10 mL) and water (15 mL). The organic layer was separated, washed with water (2×10 mL) and brine (10 mL), dried over anhydrous Na₂SO₄, and the solvent was removed under reduced pressure and purified by flash chromatography on silica gel to give a mixture of mono alkylated product, bis alkylated product products in 0.45 g. This mixture is used for the next reaction without further purification.

### Preparation of Photoprecursors

### General Procedure E for Coupling of Secondary Anilines with 2-aminonaphthalene-1,4-dione Via Oxalyl Linker

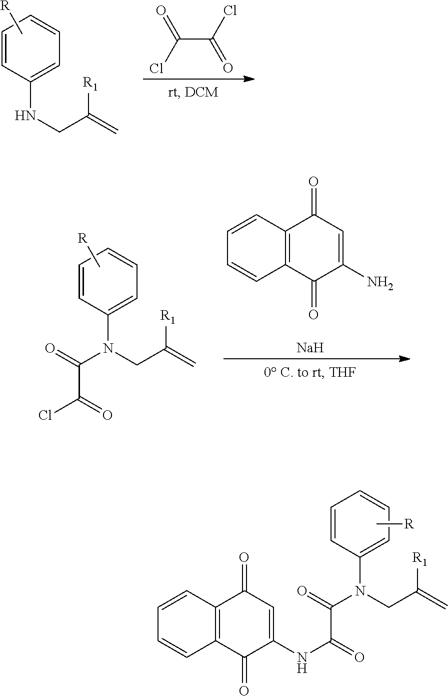

A solution of secondary amine (5.31 mmol, 1.0 eq.) in anhydrous DCM (10 mL) was quickly poured into a solution of oxalyl chloride (26.58 mmol, 5 eq., note: slow addition may lead to the formation of bis-oxalyl amide)³ in anhydrous DCM (10 mL). The reaction mixture was stirred at ambient temperature for 2 h. Upon completion of the reaction, as indicated by ¹H NMR, the volatiles was evaporated under vacuum, and the resulting residue was re-dissolved in anhydrous THF (10 mL), Under nitrogen atmosphere, a solution of 2-aminonaphthalene-1,4-dione (0.919 g, 5.31 mmol) in anhydrous THF (26 mL) sodium hydride (5.50 mmol) was added at 0° C., then allowed to stir at ambient temperature for 30 min, followed by the dropwise addition of acyl solution in THF (5.31 mmol in 10 mL of THF) at ambient temperature. The mixture was stirred at ambient temperature and monitored reaction by ¹H NMR, after that it was partitioned between ethyl acetate (50 mL) and sat. aq. NH₄Cl (10 mL). The product was extracted from an aqueous layer with ethyl acetate (3×30 mL). The combined organic layer was washed with water (3×10 mL) and brine (10 mL), and dried over anhydrous Na₂SO₄. After concentration crude product was purified by flash chromatography on silica gel to give desired photo-precursors.

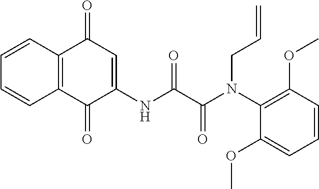 [SMILES: C=CCN(C(=O)C(=O)NC1=CC(=O)c2ccccc2C1=O)c1c(OC)cccc1OC]

N1-Allyl-N1-(2,6-dimethoxyphenyl)-N2-(1,4-dioxo-1,4-dihydronaphthalen-2-yl)oxalamide (4a): General procedure E was followed on 0.613 g (3.176 mmol) scale of secondary amine 1a. The crude product was purified by flash chromatography (SiO₂, 0-35% of ethyl acetate in hexanes) which afforded 0.92 g (69%) of the title compound 4a as a light yellow amorphous solid. ¹H NMR (500 MHz, CDCl₃) δ 9.86 (s, 1H), 8.15-8.10 (m, 1H), 8.05 (d, J=7.8 Hz, 1H), 7.78-7.69 (m, 2H), 7.65 (s, 1H), 7.28-7.23 (m, 1H), 6.59 (d, J=8.3 Hz, 2H), 5.86 (ddt, J=16.8, 9.6, 6.6 Hz, 1H), 5.15-5.04 (m, 2H), 4.31 (d, J=6.6 Hz, 2H), 3.80 (s, 6H); ¹³C {¹H}NMR (126 MHz, CDCl₃) δ 185.18, 180.46, 161.85, 159.32, 155.67, 139.75, 134.96, 133.44, 132.37, 132.32, 130.28, 129.35, 126.90, 126.45, 118.95, 118.43, 117.84, 104.62, 56.16, 52.23. HRMS (ESI) m/z: [M+Na]⁺ calcd for C₂₃H₂₀N₂O₆Na 443.1219; found 443.1249.

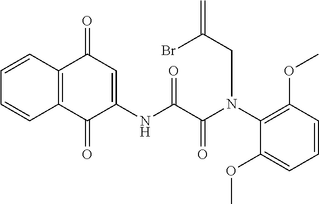 [SMILES: C=C(Br)CN(C(=O)C(=O)NC1=CC(=O)c2ccccc2C1=O)c1c(OC)cccc1OC]

N1-(2-Bromoallyl)-N1-(2,6-dimethoxyphenyl)-N2-(1,4-dioxo-1,4-dihydronaphthalen-2-yl)oxalamide (4b): General procedure E was followed on 0.680 g (2.5 mmol) scale of secondary amine 1b. The crude product was purified by flash chromatography (SiO₂, 0-35% of ethyl acetate in hexanes) which afforded 0.71 g (57%) of the title compound 4b as a light yellow amorphous solid. ¹H NMR (500 MHz, CDCl₃) δ 9.83 (s, 1H), 8.16-8.11 (m, 1H), 8.09-8.04 (m, 1H), 7.81-7.68 (m, 2H), 7.65 (s, 1H), 7.29-7.24 (m, 1H), 6.60 (d, J=8.5 Hz, 2H), 5.90 (d, J=2.0 Hz, 1H), 5.53 (d, J=1.9 Hz, 1H), 4.61 (s, 2H), 3.82 (s, 6H); ¹³C {¹H}NMR (126 MHz, CDCl₃) δ 185.15, 180.42, 162.01, 158.90, 155.38, 139.71, 135.02, 133.50, 132.31, 130.25, 129.53, 126.94, 126.48, 126.28, 119.51, 118.59, 117.97, 104.70, 56.68, 56.11. HRMS (ESI) m/z: [M+Na]⁺ calcd for C₂₃H₁₉BrN₂O₆Na 523.0307; found 523.0316.

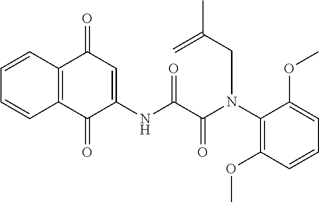 [SMILES: C=C(C)CN(C(=O)C(=O)NC1=CC(=O)c2ccccc2C1=O)c1c(OC)cccc1OC]

N1-(2,6-Dimethoxyphenyl)-N2-(1,4-dioxo-1,4-dihydronaphthalen-2-yl)-N1-(2-methylallyl)oxalamide (4c): General procedure E was followed on 0.580 g (2.8 mmol) scale of secondary amine 1c. The crude product was purified by flash chromatography (SiO₂, 0-35% of ethyl acetate in hexanes) which afforded 0.78 g (64%) of the title compound 4c as a light yellow amorphous solid. ¹H NMR (500 MHz, CDCl₃) δ 9.85 (s, 1H), 8.15-8.10 (m, 1H), 8.08-8.03 (m, 1H), 7.79-7.68 (m, 2H), 7.66 (s, 1H), 7.23 (d, J=8.4 Hz, 1H), 6.58 (d, J=8.4 Hz, 2H), 4.77-4.73 (m, 1H), 4.68 (s, 1H), 4.32 (s, 2H), 3.80 (s, 6H), 1.81 (s, 3H); ¹³C {¹H}NMR (126 MHz, CDCl₃) δ 185.19, 180.49, 162.00, 159.47, 155.70, 140.64, 139.78, 134.96, 133.44, 132.33, 130.28, 129.23, 126.90, 126.45, 118.80, 117.82, 114.61, 104.62, 56.06, 54.88, 20.82. HRMS (ESI) m/z: [M+Na]⁺ calcd for C₂₄H₂₂N₂O₆Na 457.1375; found 457.1347.

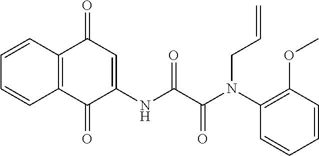 [SMILES: C=CCN(C(=O)C(=O)NC1=CC(=O)c2ccccc2C1=O)c1ccccc1OC]

N1-Allyl-N2-(1,4-dioxo-1,4-dihydronaphthalen-2-yl)-N1-(2-methoxyphenyl)oxalamide (4d): General procedure E was followed on 0.8 g (4.9 mmol) scale of secondary amine 1d. The crude product was purified by flash chromatography (SiO₂, 0-35% of ethyl acetate in hexanes) which afforded 1.24 g (64%) of the title compound 4d as a light yellow amorphous solid. ¹H NMR (500 MHz, CDCl₃) δ 9.90 (s, 1H), 8.13 (d, J=7.6 Hz, 1H), 8.08-8.03 (m, 1H), 7.80-7.69 (m, 2H), 7.63 (d, J=2.4 Hz, 1H), 7.35-7.25 (m, 1H), 7.19 (dd, J=7.8, 1.7 Hz, 1H), 7.00 (t, J=7.6 Hz, 1H), 6.92 (d, J=8.3 Hz, 1H), 5.88 (ddt, J=16.6, 10.0, 6.4 Hz, 1H), 5.21-5.14 (m, 2H), 4.49 (dd, J=14.7, 6.2 Hz, 1H), 4.23 (dd, J=14.8, 6.5 Hz, 1H), 3.77 (s, 3H); ¹³C {¹H}NMR (126 MHz, CDCl₃) δ 185.10, 180.41, 161.26, 159.08, 154.30, 139.65, 135.01, 133.49, 132.30, 131.76, 130.67, 130.23, 129.36, 127.60, 126.92, 126.48, 121.22, 118.88, 117.94, 111.86, 55.79, 53.84. HRMS (ESI) m/z: [M+Na]⁺ calcd for C₂₂H₁₆N₂O_{S}Na 413.1113; found 413.1111.

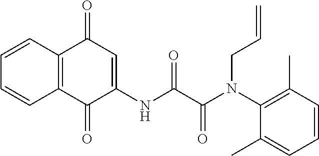 [SMILES: C=CCN(C(=O)C(=O)NC1=CC(=O)c2ccccc2C1=O)c1c(C)cccc1C]

N1-Allyl-N1-(2,6-dimethylphenyl)-N2-(1,4-dioxo-1,4-dihydronaphthalen-2-yl)oxalamide (4e): General procedure E was followed on 0.575 g (3.57 mmol) scale of secondary amine 1e. The crude product was purified by flash chromatography (SiO₂, 0-35% of ethyl acetate in hexanes) which afforded 1.2 g (86%) of the title compound 4e as a light yellow amorphous solid. ¹H NMR (500 MHz, CDCl₃) δ 10.12 (s, 1H), 8.15-8.11 (m, 1H), 8.07-8.02 (m, 1H), 7.79-7.69 (m, 2H), 7.63 (s, 1H), 7.18 (m, 1H), 7.11 (d, J=7.6 Hz, 2H), 5.98 (ddt, J=16.9, 9.7, 7.0 Hz, 1H), 5.25-5.17 (m, 2H), 4.29 (d, J=7.0 Hz, 2H), 2.22 (s, 6H); ¹³C {¹H}NMR (126 MHz, CDCl₃) δ 185.00, 180.37, 160.29, 158.31, 139.74, 139.45, 135.15, 135.03, 133.52, 132.27, 131.19, 130.21, 128.86, 128.36, 126.93, 126.48, 120.35, 118.19, 54.32, 18.72. HRMS (ESI) m/z: [M+Na]⁺ calcd for C₂₃H₂₀N₂O₄Na 411.1321; found 411.1358.

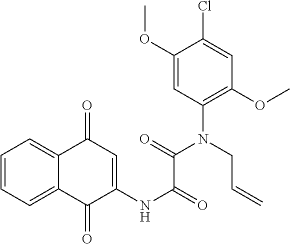 [SMILES: C=CCN(C(=O)C(=O)NC1=CC(=O)c2ccccc2C1=O)c1cc(OC)c(Cl)cc1OC]

N1-Allyl-N1-(4-chloro-2,5-dimethoxyphenyl)-N2-(1,4-dioxo-1,4-dihydronaphthalen-2-yl)oxalamide (4f): General procedure E was followed on 0.6 g (2.63 mmol) scale of secondary amine 1f. The crude product was purified by flash chromatography (SiO₂, 0-35% of ethyl acetate in hexanes) which afforded 514 mg (51%) of the title compound 4f as a light yellow amorphous solid. ¹H NMR (500 MHz, CDCl₃) δ 9.88 (s, 1H), 8.14 (d, J=7.6 Hz, 1H), 8.07 (d, J=7.6 Hz, 1H), 7.81-7.70 (m, 2H), 7.63 (s, 1H), 6.95 (s, 1H), 6.82 (s, 1H), 5.88 (ddt, J=16.9, 12.1, 6.4 Hz, 1H), 5.22-5.15 (m, 2H), 4.45 (dd, J=14.9, 6.1 Hz, 1H), 4.22 (dd, J=14.8, 6.6 Hz, 1H), 3.85 (s, 3H), 3.71 (s, 3H); ¹³C {¹H}NMR (126 MHz, CDCl₃) δ 185.01, 180.41, 161.28, 158.90, 149.52, 148.48, 139.57, 135.10, 133.55, 132.31, 131.66, 130.21, 129.59, 126.95, 126.55, 122.45, 119.22, 118.11, 114.33, 112.02, 57.02, 56.52, 54.00. HRMS (ESI) m/z: [M+Na]⁺ calcd for C₂₃H₁₉ClN₂O₆Na 477.0829; found 477.0842.

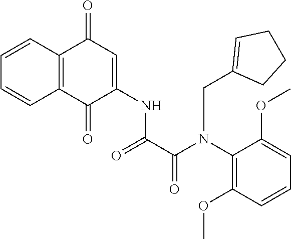 [SMILES: COc1cccc(OC)c1N(CC1=CCCC1)C(=O)C(=O)NC1=CC(=O)c2ccccc2C1=O]

N1-(Cyclopent-1-en-1-ylmethyl)-N1-(2,6-dimethoxyphenyl)-N2-(1,4-dioxo-1,4-dihydronaphthalen-2-yl)oxalamide (4g): General procedure E was followed on 0.7 g (3.0 mmol) scale of secondary amine 1g. The crude product was purified by flash chromatography (SiO₂, 0-35% of ethyl acetate in hexanes) which afforded 650 mg (47%) of the title compound 4g as a light yellow amorphous solid. ¹H NMR (500 MHz, CDCl₃) δ 9.86 (s, 1H), 8.13 (d, J=7.5 Hz, 1H), 8.05 (d, J=7.5 Hz, 1H), 7.79-7.68 (m, 2H), 7.65 (s, 1H), 7.23 (t, J=8.3 Hz, 1H), 6.57 (d, J=8.4 Hz, 2H), 5.41 (s, 1H), 4.39 (s, 2H), 3.79 (s, 6H), 2.33 (d, J=7.8 Hz, 2H), 2.20 (d, J=7.9 Hz, 2H), 1.82 (p, J=7.6 Hz, 2H); ¹³C {¹H}NMR (126 MHz, CDCl₃) δ 185.20, 180.50, 161.83, 159.49, 155.68, 139.80, 139.26, 134.94, 133.43, 132.33, 130.28, 129.36, 129.15, 126.89, 126.43, 119.06, 117.77, 104.50, 56.04, 49.15, 34.06, 32.48, 23.87. HRMS (ESI) m/z: [M+Na]⁺ calcd for C₂₆H₂₄N₂O₆Na 483.1532; found 483.1569.

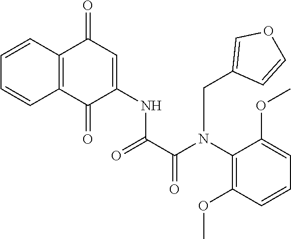 [SMILES: COc1cccc(OC)c1N(Cc1ccoc1)C(=O)C(=O)NC1=CC(=O)c2ccccc2C1=O]

N1-(2,6-Dimethoxyphenyl)-N2-(1,4-dioxo-1,4-dihydronaphthalen-2-yl)-N1-(furan-3-ylmethyl)oxalamide (4h): General procedure E was followed on 1.0 g (4.28 mmol) scale of secondary amine 1 h. The crude product was purified by flash chromatography (SiO₂, 0-35% of ethyl acetate in hexanes) which afforded 1.24 g (63%) of the title compound 4h as a light yellow amorphous solid. ¹H NMR (500 MHz, CDCl₃) δ 9.88 (s, 1H), 8.13 (d, J=7.5 Hz, 1H), 8.08-8.03 (m, 1H), 7.79-7.68 (m, 2H), 7.65 (s, 1H), 7.29-7.19 (m, 3H), 6.54 (d, J=8.4 Hz, 2H), 6.30 (d, J=1.9 Hz, 1H), 4.72 (s, 2H), 3.70 (s, 6H); ¹³C {¹H}NMR (126 MHz, CDCl₃) δ 185.16, 180.50, 162.03, 159.30, 155.60, 142.53, 141.69, 139.74, 134.97, 133.44, 132.32, 130.27, 129.37, 126.89, 126.45, 119.85, 118.75, 117.87, 111.76, 104.67, 56.07, 43.26. HRMS (ESI) m/z: [M+Na]⁺ calcd for C₂₅H₂₀N₂O₇Na 483.1168; found 483.1185.

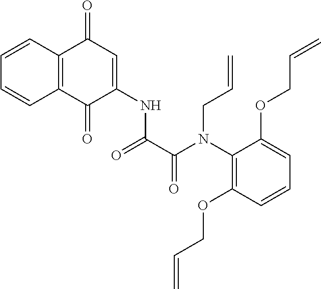 [SMILES: C=CCOc1cccc(OCC=C)c1N(CC=C)C(=O)C(=O)NC1=CC(=O)c2ccccc2C1=O]

N1-Allyl-N1-(2,6-bis(allyloxy)phenyl)-N2-(1,4-dioxo-1,4-dihydronaphthalen-2-yl)oxalamide (4i): General procedure E was followed on 580 mg (2.36 mmol) scale of secondary amine 1k. The crude product was purified by flash chromatography (SiO₂, 0-35% of ethyl acetate in hexanes) which afforded 725 mg (65%) of the title compound 4i as a light yellow amorphous solid. ¹H NMR (500 MHz, CDCl₃) δ 9.89 (s, 1H), 8.13 (dd, J=7.5, 1.5 Hz, 1H), 8.05 (dd, J=7.5, 1.5 Hz, 1H), 7.81-7.68 (m, 2H), 7.64 (s, 1H), 7.20 (t, J=8.4 Hz, 1H), 6.57 (d, J=8.4 Hz, 2H), 6.01-5.84 (m, 3H), 5.35 (q, J=1.6 Hz, 1H), 5.32 (q, J=1.7 Hz, 1H), 5.23 (q, J=1.5 Hz, 1H), 5.21 (q, J=1.4 Hz, 1H), 5.13 (dq, J=17.1, 1.5 Hz, 1H), 5.07 (dq, J=10.1, 1.2 Hz, 1H), 4.53 (dq, J=4.9, 1.5 Hz, 4H), 4.36 (dt, J=6.6, 1.3 Hz, 2H); ¹³C {¹H}NMR (126 MHz, CDCl₃) δ 185.21, 180.33, 161.85, 159.13, 154.53, 139.79, 134.92, 133.44, 132.73, 132.33, 132.30, 130.26, 129.03, 126.90, 126.43, 119.62, 118.58, 117.68, 117.64, 105.85, 69.51, 52.29. HRMS (ESI) m/z: [M+Na]⁺ calcd for C₂₇H₂₄N₂O₆Na 495.1532; found 495.1553.

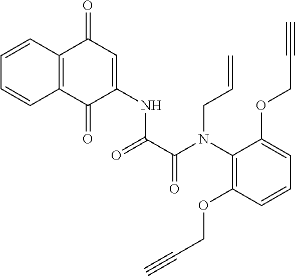 [SMILES: C#CCOc1cccc(OCC#C)c1N(CC=C)C(=O)C(=O)NC1=CC(=O)c2ccccc2C1=O]

N1-Allyl-N1-(2,6-bis(prop-2-yn-1-yloxy)phenyl)-N2-(1,4-dioxo-1,4-dihydronaphthalen-2-yl)oxalamide (4j): General procedure E was followed on 500 mg (2.07 mmol) scale of secondary amine 1l. The crude product was purified by flash chromatography (SiO₂, 0-35% of ethyl acetate in hexanes) which afforded 544 mg (56%) of the title compound 4j as a light yellow amorphous solid. ¹H NMR (500 MHz, CDCl₃) δ 9.91 (s, 1H), 8.13 (dd, J=7.5, 1.5 Hz, 1H), 8.05 (dd, J=7.5, 1.5 Hz, 1H), 7.81-7.68 (m, 2H), 7.63 (s, 1H), 7.27 (t, J=8.4 Hz, 1H), 6.74 (d, J=8.5 Hz, 2H), 5.89 (ddt, J=16.8, 10.1, 6.6 Hz, 1H), 5.14 (dq, J=17.1, 1.4 Hz, 1H), 5.09 (dq, J=10.1, 1.2 Hz, 1H), 4.70 (d, J=2.4 Hz, 4H), 4.36 (dd, J=6.7, 1.3 Hz, 2H), 2.44 (t, J=2.4 Hz, 2H); ¹³C {¹H}NMR (126 MHz, CDCl₃) δ 185.22, 180.40, 161.73, 159.03, 153.54, 139.81, 134.94, 133.47, 132.28, 132.08, 130.28, 128.92, 126.91, 126.43, 120.30, 118.96, 117.86, 106.71, 77.99, 76.14, 56.52, 52.42. HRMS (ESI) m/z: [M+Na]⁺ calcd for C₂₇H₂₀N₂O₆Na 491.1219; found 491.1234.

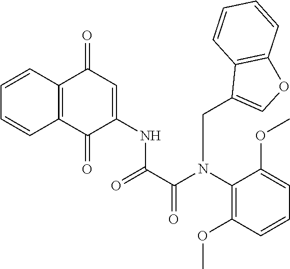 [SMILES: COc1cccc(OC)c1N(Cc1coc2ccccc12)C(=O)C(=O)NC1=CC(=O)c2ccccc2C1=O]

N1-(Benzofuran-3-ylmethyl)-N1-(2,6-dimethoxyphenyl)-N2-(1,4-dioxo-1,4-dihydronaphthalen-2-yl)oxalamide (4k): General procedure E was followed on 600 mg (2.12 mmol) scale of secondary amine 1i. The crude product was purified by flash chromatography (SiO₂, 0-35% of ethyl acetate in hexanes) which afforded 512 mg (48%) of the title compound 4k as a light yellow amorphous solid. ¹H NMR (500 MHz, CDCl₃) δ 9.91 (s, 1H), 8.15-8.10 (m, 1H), 8.07-8.02 (m, 1H), 7.80-7.68 (m, 2H), 7.64 (s, 1H), 7.42-7.38 (m, 3H), 7.26-7.15 (m, 3H), 6.47 (d, J=8.5 Hz, 2H), 5.00 (d, J=0.8 Hz, 2H), 3.45 (s, 6H); ¹³C {¹H}NMR (126 MHz, CDCl₃) δ 185.13, 180.50, 161.91, 159.24, 155.79, 155.21, 144.63, 139.68, 134.97, 133.44, 132.30, 130.24, 129.55, 127.62, 126.87, 126.44, 124.27, 122.64, 120.54, 118.47, 117.92, 115.68, 111.18, 104.59, 55.83, 41.47; HRMS (ESI) m/z: [M+Na]⁺ calcd for C₂₉H₂₂N₂O₇Na 533.1324; found 533.1313.

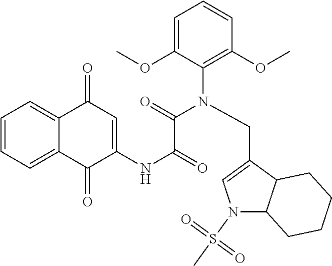 [SMILES: COc1cccc(OC)c1N(CC1=CN(S(C)(=O)=O)C2CCCCC12)C(=O)C(=O)NC1=CC(=O)c2ccccc2C1=O]

N1-(2,6-Dimethoxyphenyl)-N2-(1,4-dioxo-1,4-dihydronaphthalen-2-yl)-N1-((1-(methyl sulfonyl)-1H-indol-3-yl)methyl)oxalamide (41): General procedure E was followed on 600 mg (1.66 mmol) scale of secondary amine 1m. The crude product was purified by flash chromatography (SiO₂, 0-35% of ethyl acetate in hexanes) which afforded 665 mg (68%) of the title compound 41 as a light yellow amorphous solid. ¹H NMR (500 MHz, CDCl₃) δ 9.92 (s, 1H), 8.16-8.10 (m, 1H), 8.05 (d, J=7.4 Hz, 1H), 7.83 (d, J=8.0 Hz, 1H), 7.79-7.69 (m, 2H), 7.62 (d, J=14.3 Hz, 2H), 7.32 (dt, J=17.4, 7.2 Hz, 2H), 7.21-7.14 (m, 2H), 6.45 (d, J=8.4 Hz, 2H), 5.07 (s, 2H), 3.49 (s, 6H), 2.94 (s, 3H); ¹³C {¹H}NMR (126 MHz, CDCl₃) δ 185.14, 180.50, 161.85, 159.13, 155.70, 139.65, 135.08, 135.01, 133.48, 132.28, 130.47, 130.22, 129.61, 126.89, 126.47, 125.99, 125.08, 123.63, 120.79, 118.10, 117.97, 117.62, 112.90, 104.45, 55.86, 42.44, 40.45. HRMS (ESI) m/z: [M+Na]⁺ calcd for C₃₀H₂₅N₃O₈SNa 610.0627, found 610.0627.

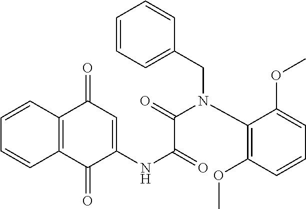 [SMILES: COc1cccc(OC)c1N(Cc1ccccc1)C(=O)C(=O)NC1=CC(=O)c2ccccc2C1=O]

N1-Benzyl-N1-(2,6-dimethoxyphenyl)-N2-(1,4-dioxo-1,4-dihydronaphthalen-2-yl)oxalamide (4m): General procedure E was followed on 750 mg (3.08 mmol) scale of secondary amine in. The crude product was purified by flash chromatography (SiO₂, 0-35% of ethyl acetate in hexanes) which afforded 565 mg (71%) of the title compound 4m as a light yellow amorphous solid. ¹H NMR (500 MHz, CDCl₃) δ 9.93 (s, 1H), 8.18 (d, J=7.6 Hz, 1H), 8.10 (d, J=7.5 Hz, 1H), 7.84-7.73 (m, 2H), 7.70 (s, 1H), 7.33-7.30 (m, 3H), 7.29-7.22 (m, 3H), 6.54 (d, J=8.3 Hz, 2H), 4.93 (s, 2H), 3.66 (s, 6H); ¹³C {¹H}NMR (126 MHz, CDCl₃) δ 185.17, 180.49, 162.00, 159.47, 155.62, 139.75, 136.16, 134.95, 133.43, 132.32, 130.26, 129.79, 129.38, 127.93, 127.63, 126.88, 126.43, 118.69, 117.87, 104.62, 56.04, 52.45. HRMS (ESI) m/z: [M+Na]⁺ calcd for C₂₇H₂₂N₂O₆Na 493.1375; found 493.1373.

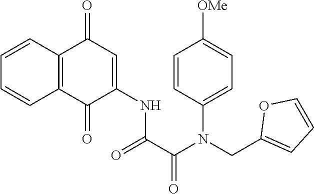 [SMILES: COc1ccc(N(Cc2ccco2)C(=O)C(=O)NC2=CC(=O)c3ccccc3C2=O)cc1]

N1-(1,4-Dioxo-1,4-dihydronaphthalen-2-yl)-N2-(furan-2-ylmethyl)-N2-(4-methoxyphenyl) oxalamide (4n): General procedure E was followed on 550 mg (2.7 mmol) scale of N-(Furan-2-ylmethyl)-4-methoxyaniline. The crude product was purified by flash chromatography (SiO₂, 0-35% of ethyl acetate in hexanes) which afforded 565 mg (49%) of the title compound 4n as a light yellow amorphous solid. ¹H NMR (500 MHz, CDCl₃) δ 10.01 (s, 1H), 8.15 (dd, J=7.5, 1.6 Hz, 1H), 8.08 (dd, J=7.6, 1.5 Hz, 1H), 7.83-7.71 (m, 2H), 7.64 (s, 1H), 7.41 (dd, J=1.9, 0.8 Hz, 1H), 7.02-6.98 (m, 2H), 6.92-6.85 (m, 2H), 6.33 (dd, J=3.2, 1.9 Hz, 1H), 6.24 (d, J=3.3 Hz, 1H), 4.94 (s, 2H), 3.84 (s, 3H); ¹³C {¹H}NMR (126 MHz, CDCl₃) δ 185.03, 180.35, 160.31, 159.44, 158.76, 149.13, 142.85, 139.45, 135.05, 133.87, 133.54, 132.27, 130.18, 128.10, 126.95, 126.50, 118.17, 114.60, 110.64, 110.24, 55.51, 48.07. HRMS (ESI) m/z: [M+Na]⁺ calcd for C₂₄H₁₈N₂O₆Na 453.1063; found 453.1030.

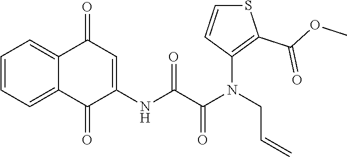 [SMILES: C=CCN(C(=O)C(=O)NC1=CC(=O)c2ccccc2C1=O)c1ccsc1C(=O)OC]

Methyl 3-(N-allyl-2-((1,4-dioxo-1,4-dihydronaphthalen-2-yl)amino)-2-oxoacetamido) thiophene-2-carboxylate (4p): General procedure E was followed on 1.2 g (3.04 mmol) scale of secondary amine 1o The crude product was purified by flash chromatography (SiO₂, 0-35% of ethyl acetate in hexanes) which afforded 650 mg (50%) of the title compound 4p as a light yellow amorphous solid. ¹H NMR (500 MHz, CDCl₃) δ 10.02 (s, 1H), 8.14 (dd, J=7.6, 1.5 Hz, 1H), 8.06 (dd, J=7.5, 1.5 Hz, 1H), 7.80-7.69 (m, 2H), 7.61 (s, 1H), 7.55 (d, J=5.3 Hz, 1H), 7.06 (d, J=5.3 Hz, 1H), 5.88 (ddt, J=16.8, 10.3, 6.5 Hz, 1H), 5.21-5.12 (m, 2H), 4.42 (dd, J=14.7, 6.7 Hz, 1H), 4.30 (dd, J=14.8, 6.3 Hz, 1H), 3.78 (s, 3H); ¹³C {¹H}NMR (126 MHz, CDCl₃) δ 185.13, 180.23, 161.79, 160.61, 158.77, 144.36, 139.63, 134.99, 133.53, 132.27, 131.26, 130.58, 130.24, 127.45, 126.96, 126.47, 124.87, 119.56, 118.06, 53.96, 52.38. HRMS (ESI) m/z: [M+Na]⁺ calcd for C₂₁H₁₆N₂O₆SNa 447.0627; found 447.0659.

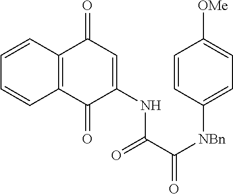 [SMILES: COc1ccc(N(Cc2ccccc2)C(=O)C(=O)NC2=CC(=O)c3ccccc3C2=O)cc1]

N′-Benzyl-N-(1,4-dioxo-1,4-dihydronaphthalen-2-yl)-N′-(4-methoxyphenyl)ethanediamide (1s): General procedure F was followed on a 0.685 mmol scale of 2-amino-1,4-naphthalenedione 3 with 1.37 mmol of [benzyl(4-methoxyphenyl)carbamoyl]carbonyl chloride⁷, which after flash chromatography on silica gel (gradient elution: hexanes/hexane-ethyl acetate+1% v/v Et₃N 20:80) afforded 0.156 g (52%) of the title compound. ¹H NMR (500 MHz, CDCl₃) δ 9.97 (br s, 1H), 8.13 (dd, J=7.5, 1.2 Hz, 1H), 8.05 (dd, J=7.6, 1.1 Hz, 1H), 7.77 (td, J=7.5, 1.4 Hz, 1H), 7.72 (td, J=7.5, 1.4 Hz, 1H), 7.62 (s, 1H), 7.33-7.27 (m, 3H), 7.25-7.20 (m, 2H), 6.94-6.88 (m, 2H), 6.85-6.79 (m, 2H), 4.94 (s, 2H), 3.79 (s, 3H). ¹³_{C} NMR (126 MHz, CDCl₃) δ 185.0, 180.4, 160.5, 159.3, 159.1, 139.4, 135.8, 135.1, 134.0, 133.5, 132.2, 130.1, 129.2, 128.7, 128.2, 128.1, 126.9, 126.5, 118.1, 114.5, 55.6, 55.5. HRMS (ESI) m/z: [M+H]⁺ calcd for C₂₆H₂₁N₂O₅441.1445, found 441.1442.

### General Procedure F for Acylation of 2-Amino-1,4-naphthoquinone

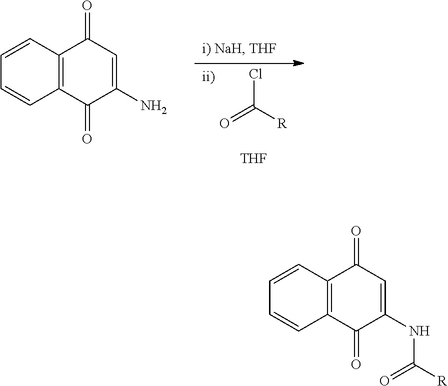

To a solution of 2-amino-1,4-naphthalenedione (0.115 g, 0.667 mmol) in distilled THF (2 mL), sodium hydride (80 mg of 60% suspension in oil, 48 mg, 2.0 mmol) was added, and the mixture was stirred at ambient temperature for 1 h. After that, a solution of acyl chloride (1.00 mmol) in distilled THF (4 mL) was added dropwise, and the mixture was refluxed overnight. The reaction was then carefully quenched with water (5 mL), and the product was extracted with DCM (3×20 mL). The combined organic layers were dried over anhydrous Na₂SO₄, filtered, and the solvent was removed under reduced pressure. Further purification by flash chromatography on silica gel yielded the desired photoprecursor.

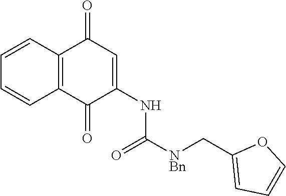 [SMILES: O=C1C=C(NC(=O)N(Cc2ccccc2)Cc2ccco2)C(=O)c2ccccc21]

1-Benzyl-3-(1,4-dioxo-1,4-dihydronaphthalen-2-yl)-1-(furan-2-ylmethyl)urea (40): General procedure F was followed on a 0.515 mmol scale of 2-amino-1,4-naphthalenedione with 1.03 mmol of N-benzyl-N-[(furan-2-yl)methyl]carbamoyl chloride, which after flash chromatography (SiO2, 0-35% of ethyl acetate in hexanes+1% v/v Et3N 75:25) afforded 0.153 g (77%) of the title compound 4o. 1H NMR (500 MHz, CDCl3) δ 8.19 (s, 1H), 8.09 (d, J=7.7 Hz, 1H), 8.04 (d, J=7.7 Hz, 1H), 7.75 (t, J=7.5 Hz, 1H), 7.71-7.64 (m, 2H), 7.46-7.42 (m, 1H), 7.41-7.35 (m, 2H), 7.34-7.29 (m, 3H), 6.37 (d, J=1.4 Hz, 2H), 4.67 (s, 2H), 4.54 (s, 2H); 13C{1H}NMR (126 MHz, CDCl3) δ 185.01, 181.58, 153.79, 149.9, 143.18, 141.73, 134.98, 133.02, 132.47, 130.19, 129.14, 128.16, 127.68, 126.65, 126.48, 115.26, 110.69, 109.22, 50.72, 43.72. HRMS (ESI) m/z: [M+H]+ calcd for C23H19N2O4 387.1339; found 387.1343.

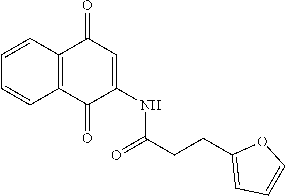 [SMILES: O=C(CCc1ccco1)NC1=CC(=O)c2ccccc2C1=O]

N-(1,4-Dioxo-1,4-dihydronaphthalen-2-yl)-3-(furan-2-yl)propenamide (4r): General procedure F was followed on a 0.667 mmol scale of 2-amino-1,4-naphthalenedione with 1.00 mmol of 3-(furan-2-yl)propanoyl chloride4, which after flash chromatography on silica gel (eluent: chloroform+1% v/v Et3N) afforded 0.114 g (58%) of the title compound 4r. ¹H NMR (500 MHz, CDCl3) δ 8.35 (br s, 1H), 8.10 (dd, J=7.7, 1.1 Hz, 2H), 7.86 (s, 1H), 7.79 (td, J=7.5, 1.4 Hz, 1H), 7.72 (td, J=7.3, 1.1 Hz, 1H), 7.33 (d, J=1.9 Hz, 1H), 6.29 (dd, J=3.2, 2.0 Hz, 1H), 6.08 (d, J=2.9 Hz, 1H), 3.08 (t, J=7.3 Hz, 2H), 2.84 (t, J=7.4 Hz, 2H). 13C NMR (126 MHz, CDCl3) δ 185.4, 181.1, 171.3, 153.4, 141.6, 139.9, 135.1, 133.4, 132.2, 130.0, 126.8, 126.5, 117.4, 110.5, 106.1, 36.3, 23.4. HRMS (ESI) m/z: [M+H]+ calcd for C17H14NO4296.0917; found 296.0923.

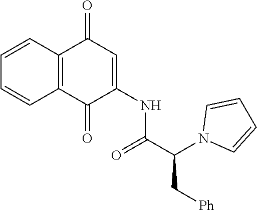 [SMILES: O=C1C=C(NC(=O)[C@H](Cc2ccccc2)n2cccc2)C(=O)c2ccccc21]

(2S)—N-(1,4-Dioxo-1,4-dihydronaphthalen-2-yl)-3-phenyl-2-(1H-pyrrol-1-yl)propenamide (1r): Under argon, (2S)-3-phenyl-2-(pyrrol-1-yl)propanoic acid6 (0.215 g, 1.00 mmol) was dissolved in anhydrous DMF (0.83 mL), the solution was treated with HOAt (0.163 g, 1.20 mmol), HATU (0.456 g, 1.20 mmol) and DIPEA (0.43 mL, 0.32 g, 2.5 mmol) and then stirred at ambient temperature for 10 min. Subsequently, 2-amino-1,4-naphthalenedione 57440 (0.208 g, 1.20 mmol) was added, and the mixture was stirred at ambient temperature for 1 day. Further removal of DMF in vacuo followed by flash chromatography on silica gel (eluent: DCM) afforded 0.244 g (66%) of the title compound 1r. ¹H NMR (500 MHz, CDCl3) δ 8.34 (s, 1H), 8.08 (ddd, J=7.7, 1.3, 0.5 Hz, 1H), 8.02 (ddd, J=7.7, 1.4, 0.5 Hz, 1H), 7.88 (s, 1H), 7.76 (td, J=7.6, 1.4 Hz, 1H), 7.69 (td, J=7.6, 1.3 Hz, 1H), 7.26-7.19 (m, 3H), 7.10-7.04 (m, 2H), 6.76 (t, J=2.1 Hz, 2H), 6.37-6.33 (m, 2H), 4.89 (dd, J=10.5, 4.5 Hz, 1H), 3.72 (dd, J=14.5, 4.5 Hz, 1H), 3.33 (dd, J=14.5, 10.5 Hz, 1H). 13C NMR (126 MHz, CDCl3) δ 185.2, 180.6, 170.1, 139.6, 136.7, 135.2, 133.5, 132.2, 129.9, 128.9, 128.8, 127.2, 126.9, 126.5, 120.3, 117.8, 110.9, 66.1, 37.8. HRMS (ESI) m/z: [M+H]+ calcd for C23H19N2O3 371.1390; found 371.1391.

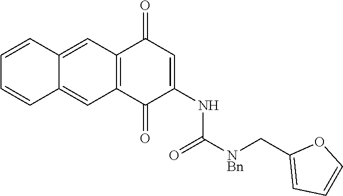 [SMILES: O=C1C=C(NC(=O)N(Cc2ccccc2)Cc2ccco2)C(=O)c2cc3ccccc3cc21]

1-Benzyl-3-(1,4-dioxo-1,4-dihydroanthracen-2-yl)-1-[(furan-2-yl)methyl]urea (4q): To a solution of 2-amino-1,4-anthracenedione (0.149 g, 0.668 mmol) in distilled THF (2 mL), sodium hydride (80 mg of 60% suspension in oil, 48 mg, 2.0 mmol) was added, and the mixture was stirred at ambient temperature for 40 min. After that, solution of N-benzyl-N-[(furan-2-yl)methyl]carbamoyl chloride (1.00 mmol) in distilled THF (3 mL) was added dropwise, followed by addition of anhydrous DMF (1 mL) and a second portion of sodium hydride (80 mg of 60% suspension in oil, 48 mg, 2.0 mmol). The mixture was refluxed overnight, and then the reaction was carefully quenched with water (5 mL). Removal of solvents in vacuo and subsequent purification by flash chromatography on silica gel (gradient elution: hexanes/ethyl acetate) afforded 0.134 g (46%) of the title compound 4q (combined fractions were washed with 1N HCl to remove benzyl(furan-2-ylmethyl)amine). ¹H NMR (500 MHz, CDCl₃) δ 8.63-8.56 (m, 2H), 8.32 (br s, 1H), 8.05 (d, J=7.9 Hz, 1H), 8.02 (d, J=7.6 Hz, 1H), 7.80 (s, 1H), 7.72-7.62 (m, 2H), 7.45 (dd, J=1.8, 0.9 Hz, 1H), 7.43-7.36 (m, 2H), 7.36-7.24 (m, 3H), 6.39 (dd, J=3.3, 0.8 Hz, 1H), 6.38 (dd, J=3.3, 1.8 Hz, 1H), 4.69 (s, 2H), 4.57 (br s, 2H). ¹³_{C} NMR (126 MHz, CDCl₃) δ 184.7, 181.1, 153.8, 150.0, 143.2, 143.0, 135.7, 134.5, 130.4, 130.3, 130.0, 129.6, 129.4, 129.2, 128.6, 128.5, 128.2, 127.9, 127.7, 126.9, 117.0, 110.7, 109.3, 50.7, 43.7. HRMS (ESI) m/z: [M−H]⁺ calcd for C₂₇H₁₉N₂O₄435.1350; found 435.1342.

### Photoreactions

Irradiation procedure (G): photoprecursor (0.4 mmol) was dissolved in DMSO (200 mL), and the solution was degassed by bubbling nitrogen or argon for 1 h. The mixture was irradiated with two visible light LEDs, (High Power LED Star #A008-UV410-48; λₘₓ=410-420 nm), with reaction progress monitored by 1H NMR. DMSO was evaporated under vacuum (60° C./0.15 Torr). Crude photoproduct (0.4 mmol) was dissolved in anhydrous DCM (20 mL), and manganese (IV) oxide (174 mg, 2.0 mmol, 5eq) was added. The mixture was stirred at ambient temperature and the progress of the reaction was monitored by 1H NMR. Upon completion, the mixture was filtered through celite bed and washed bed with DCM (2×10 mL). The resulted filtrate was washed with water (2×20 mL). Combined organic layers were dried over anhydrous Na2SO4 and concentrated in vacuo. The crude residue was dissolved in minimum amount of DCM followed by the addition Hexanes, resulted solid was filtered and solid was washed with 5% DCM in Hexanes solution.

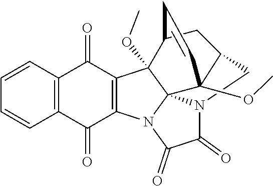 [SMILES: CO[C@@]12C=C[C@H]3C[C@@H]1CN1C(=O)C(=O)N4C5=C(C(=O)c6ccccc6C5=O)[C@]3(OC)[C@]142]

(2R,2aS,2a1R,5R,5aS)-2a,5a-Dimethoxy-2,2a,5,5a-tetrahydro-1H-11b,13a-diaza-2,5-methanobenzo[b]pentaleno[1,6-jk]fluorene-6,11,12,13-tetraone (6a): General procedure G was followed on 202 mg (0.48 mmol) scale of the photoprecursor 4a. After the photochemical reaction afford 175 mg (88%) of product 6a. ¹H NMR (500 MHz, CDCl3) δ 8.20-8.15 (m, 1H), 8.10-8.05 (m, 1H), 7.81-7.75 (m, 2H), 6.26 (t, J=7.9 Hz, 1H), 6.20 (d, J=8.9 Hz, 1H), 3.90 (dd, J=11.3, 5.6 Hz, 1H), 3.78-3.73 (m, 1H), 3.41-3.35 (m, 4H), 3.33 (s, 3H), 2.68-2.59 (m, 1H), 2.15 (d, J=13.4 Hz, 1H), 1.94 (t, J=12.3 Hz, 1H); 13C {¹H}NMR (126 MHz, CDCl3) δ 182.39, 176.53, 160.79, 160.09, 147.24, 134.66, 134.47, 134.37, 134.35, 132.08, 132.03, 127.34, 127.32, 126.64, 88.51, 86.07, 82.38, 53.39, 52.86, 49.94, 37.56, 32.92, 32.26. HRMS (ESI) m/z: [M+Na]+ calcd for C23H18N2O6Na 441.1063; found 441.1059.

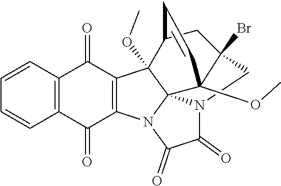 [SMILES: CO[C@@]12C=C[C@H]3C[C@]1(Br)CN1C(=O)C(=O)N4C5=C(C(=O)c6ccccc6C5=O)[C@]3(OC)[C@]142]

(2S,2aR,2a1S,5R,5aS)-2-Bromo-2a,5a-dimethoxy-2,2a,5,5a-tetrahydro-1H-11b,13a-diaza-2,5-methanobenzo[b]pentaleno[1,6-jk]fluorene-6,11,12,13-tetraone (6b): General procedure G was followed on 200 mg (0.40 mmol) scale of the photoprecursor 4b. After the photochemical reaction afford 168 mg (84%) of product 6b. 1H NMR (500 MHz, CDCl3) δ 8.22-8.15 (m, 1H), 8.13-8.05 (m, 1H), 7.85-7.76 (m, 2H), 6.44 (dd, J=9.0, 7.0 Hz, 1H), 6.37 (dd, J=9.0, 1.4 Hz, 1H), 4.18 (d, J=11.0 Hz, 1H), 3.83 (d, J=11.1 Hz, 1H), 3.71 (dtd, J=7.0, 2.8, 1.3 Hz, 1H), 3.65 (s, 3H), 3.32 (s, 3H), 2.86 (dd, J=14.5, 2.7 Hz, 1H), 2.46 (ddd, J=14.5, 2.8, 1.3 Hz, 1H); 13C{1H}NMR (126 MHz, CDCl3) δ 182.16, 176.38, 160.08, 158.84, 147.02, 134.88, 134.57, 134.34, 134.19, 132.03, 132.01, 127.41, 126.81, 124.37, 87.89, 85.19, 84.25, 61.44, 58.04, 56.67, 53.42, 44.40, 33.96. HRMS (ESI) m/z: [M+Na]+ calcd for C23H17BrN2O6Na 521.0151; found 521.0172.

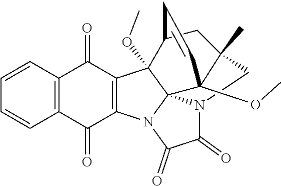 [SMILES: CO[C@@]12C=C[C@H]3C[C@]1(C)CN1C(=O)C(=O)N4C5=C(C(=O)c6ccccc6C5=O)[C@]3(OC)[C@]142]

(2R,2aS,2a1R,5R,5aS)-2a,5a-Dimethoxy-2-methyl-2,2a,5,5a-tetrahydro-1H-11b,13a-diaza-2,5-methanobenzo[b]pentaleno[1,6-jk]fluorene-6,11,12,13-tetraone (6c): General procedure G was followed on 200 mg (0.46 mmol) scale of the photoprecursor 4c. After the photochemical reaction afford 170 mg (85%) of product 6c. 1H NMR (500 MHz, CDCl3) δ 8.19-8.13 (m, 1H), 8.08 (dd, J=6.5, 3.0 Hz, 1H), 7.79 (dd, J=9.1, 5.0 Hz, 2H), 6.31-6.26 (m, 2H), 3.72-3.67 (m, 1H), 3.64 (d, J=11.0 Hz, 1H), 3.51 (s, 3H), 3.37 (d, J=10.8 Hz, 1H), 3.32 (s, 3H), 2.31 (dd, J=13.4, 2.9 Hz, 1H), 1.54-1.48 (m, 1H), 1.02 (s, 3H); 13C{1H}NMR (126 MHz, CDCl3) δ 182.34, 176.69, 160.31, 160.00, 146.93, 135.23, 134.71, 134.37, 133.78, 132.13, 132.01, 127.26, 126.71, 125.35, 89.86, 85.88, 85.02, 56.27, 55.52, 53.17, 47.73, 40.04, 33.05, 19.92. HRMS (ESI) m/z: [M+Na]+ calcd for C24H20N2O6Na 455.1219; found 455.1213.

General procedure G was followed on 200 mg (0.51 mmol) scale of the photoprecursor 4d. After the photochemical reaction afford 120 mg (60%) of major product 6d and 30 mg (15%) of minor product 6da.

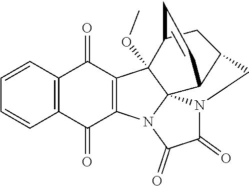 [SMILES: CO[C@]12C3=C(C(=O)c4ccccc4C3=O)N3C(=O)C(=O)N4C[C@H]5C[C@@H]1C=C[C@H]5[C@@]432]

(2S,2aR,2a1R,5R,5aS)-5a-Methoxy-2,2a,5,5a-tetrahydro-1H-11b,13a-diaza-2,5-methanobenzo[b]pentaleno[1,6-jk]fluorene-6,11,12,13-tetraone (6d): 1H NMR (500 MHz, CDCl3) δ 8.20-8.14 (m, 1H), 8.10-8.05 (m, 1H), 7.82-7.75 (m, 2H), 6.25 (t, J=7.7 Hz, 1H), 6.08 (t, J=7.2 Hz, 1H), 3.91 (dd, J=11.7, 5.5 Hz, 1H), 3.86-3.81 (m, 1H), 3.51 (d, J=11.6 Hz, 1H), 3.34 (s, 3H), 2.73 (t, J=5.3 Hz, 1H), 2.59 (dq, J=11.0, 5.3 Hz, 1H), 2.06-1.97 (m, 1H), 1.89-1.81 (m, 1H); 13C{1H}NMR (126 MHz, CDCl3) δ 182.63, 176.95, 161.18, 158.53, 147.16, 136.07, 135.28, 134.67, 134.35, 132.09, 131.95, 127.81, 127.28, 126.69, 86.80, 85.83, 53.38, 50.93, 46.33, 35.37, 32.74, 30.70. HRMS (ESI) m/z: [M+Na]+ calcd for C22H16N2O5Na 411.0957; found 411.0955.

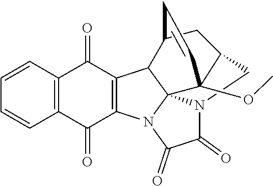 [SMILES: CO[C@@]12C=C[C@H]3C[C@@H]1CN1C(=O)C(=O)N4C5=C(C(=O)c6ccccc6C5=O)C3[C@]142]

(2R,2aS,2a1S,5R)-2a-Methoxy-2,2a,5,5a-tetrahydro-1H-11b,13a-diaza-2,5-methanobenzo[b]pentaleno[1,6-jk]fluorene-6,11,12,13-tetraone (6da): 1H NMR (500 MHz, CDCl3) δ 8.15 (dd, J=6.1, 3.1 Hz, 1H), 8.06-8.00 (m, 1H), 7.75 (dd, J=5.9, 3.2 Hz, 2H), 6.31 (d, J=8.8 Hz, 1H), 6.24 (dd, J=8.9, 6.5 Hz, 1H), 3.87 (dd, J=11.5, 5.5 Hz, 1H), 3.83 (d, J=4.2 Hz, 1H), 3.69-3.63 (m, 1H), 3.43 (s, 3H), 3.26 (d, J=11.5 Hz, 1H), 2.70-2.60 (m, 1H), 2.12 (ddd, J=13.5, 10.9, 2.6 Hz, 1H), 1.88 (dd, J=13.5, 2.8 Hz, 1H); 13C{1H}NMR (126 MHz, CDCl3) δ 182.20, 176.04, 162.15, 159.14, 145.84, 136.26, 135.17, 134.39, 134.22, 132.09, 131.98, 127.28, 126.31, 126.23, 89.15, 80.85, 52.90, 51.98, 49.83, 36.84, 36.06, 29.81. HRMS (ESI) calcd for C24H20N2O6Na+ (MNa+) 455.1219, found 455.1213; HRMS (ESI) m/z: [M+Na]+ calcd for C22H16N2O5Na 411.0957; found 411.0916.

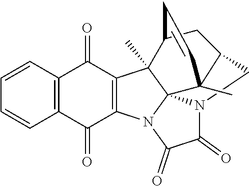 [SMILES: C[C@]12C3=C(C(=O)c4ccccc4C3=O)N3C(=O)C(=O)N4C[C@H]5C[C@@H]1C=C[C@@]5(C)[C@@]432]

(2S,2aR,2a1R,5R,5aR)-2a,5a-Dimethyl-2,2a,5,5a-tetrahydro-1H-11b,13a-diaza-2,5-methanobenzo[b]pentaleno[1,6-jk]fluorene-6,11,12,13-tetraone (6e): General procedure G was followed on 200 mg (0.515 mmol) scale of the photoprecursor 4e. After the photochemical reaction afford 152 mg (76%) of product 6e. 1H NMR (500 MHz, CDCl3) δ 8.20-8.15 (m, 1H), 8.08-8.03 (m, 1H), 7.79-7.76 (m, 2H), 6.34 (dd, J=8.2, 6.8 Hz, 1H), 5.83 (dd, J=8.2, 1.5 Hz, 1H), 3.88 (dd, J=12.2, 5.6 Hz, 1H), 3.40 (dt, J=7.8, 2.7 Hz, 1H), 3.35 (d, J=12.2 Hz, 1H), 2.24 (m, 1H), 2.02-1.90 (m, 2H), 1.30 (s, 3H), 1.10 (s, 3H); 13C{1H}NMR (126 MHz, CDCl3) δ 181.95, 176.41, 160.50, 160.17, 143.38, 143.30, 137.32, 134.35, 134.19, 132.27, 131.88, 131.83, 127.15, 126.39, 90.41, 53.44, 49.85, 45.77, 40.74, 35.87, 33.82, 18.88, 15.74. HRMS (ESI) m/z: [M+H]+ calcd for C23H19N2O4 387.1339; found 387.1335.

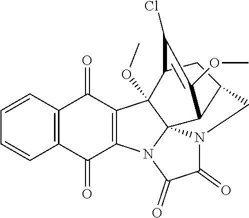 [SMILES: COC1=C(Cl)[C@H]2C[C@@H]3CN4C(=O)C(=O)N5C6=C(C(=O)c7ccccc7C6=O)[C@]2(OC)[C@]45[C@H]13]

(2S,2aR,2a1R,5S,5aS)-4-Chloro-3,5a-dimethoxy-2,2a,5,5a-tetrahydro-1H-11b,13a-diaza-2,5-methanobenzo[b]pentaleno[1,6-jk]fluorene-6,11,12,13-tetraone (6f): General procedure G was followed on 200 mg (0.44 mmol) scale of the photoprecursor 4f. After the photochemical reaction afford 132 mg (66%) of product 6f. ¹H NMR (500 MHz, CDCl3) δ 8.24-8.20 (m, 1H), 8.19-8.14 (m, 1H), 7.88-7.80 (m, 2H), 3.97 (dd, J=11.8, 5.3 Hz, 1H), 3.85 (t, J=2.8 Hz, 1H), 3.68 (s, 3H), 3.54 (d, J=11.7 Hz, 1H), 3.40 (s, 3H), 2.88-2.84 (m, 1H), 2.89-2.80 (m, 1H), 2.24-2.15 (m, 1H), 2.13-2.05 (m, 1H); 13C{1H}NMR (126 MHz, CDCl3) δ 182.10, 176.69, 160.77, 158.04, 147.48, 147.36, 135.04, 134.89, 134.45, 132.10, 131.87, 127.32, 126.99, 105.73, 86.74, 86.68, 58.35, 54.08, 50.55, 49.51, 41.24, 36.13, 32.05. HRMS (ESI) m/z: [M+Na]+ calcd for C23H17ClN2O6Na 475.0673; found 475.0653.

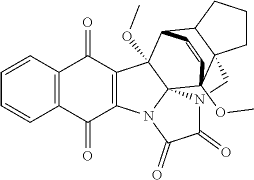 [SMILES: CO[C@@]12C=C[C@H]3C4CCC[C@]41CN1C(=O)C(=O)N4C5=C(C(=O)c6ccccc6C5=O)[C@]3(OC)[C@]142]

(2a1R,3aR,7S,7aS,16S)-7a,16-Dimethoxy-4,5,6,6a,7,7a-hexahydro-3H-2a,13b-diaza-2a1,3a,7-(epiprop[2]ene[1,1,3]triyl)dicyclopenta[cd,g]naphtho[2,3-a]azulene-1,2,8,13-tetraone (6g): General procedure G was followed on 200 mg (0.434 mmol) scale of the photoprecursor 4g. After the photochemical reaction afford 166 mg (83%) of product 6g. ¹H NMR (500 MHz, CDCl3) δ 8.20-8.13 (m, 1H), 8.12-8.04 (m, 1H), 7.84-7.76 (m, 2H), 6.31 (d, J=9.2 Hz, 1H), 6.21 (dd, J=9.0, 6.9 Hz, 1H), 3.80 (q, J=5.7 Hz, 2H), 3.51 (s, 3H), 3.42 (d, J=10.7 Hz, 1H), 3.33 (s, 3H), 2.70 (td, J=8.8, 2.9 Hz, 1H), 2.07-1.98 (m, 1H), 1.80-1.55 (m, 3H), 1.52-1.42 (m, 1H), 1.34-1.22 (m, 1H); 13C{1H}NMR (126 MHz, CDCl3) δ 182.38, 176.74, 160.58, 159.91, 146.71, 135.33, 134.70, 134.38, 133.64, 132.13, 132.01, 127.27, 126.73, 126.71, 89.95, 86.52, 85.35, 59.66, 55.93, 55.06, 53.17, 51.43, 37.39, 32.28, 32.19, 26.27. HRMS (ESI) m/z: [M+Na]+ calcd for C26H22N2O6Na 481.1375; found 481.1348.

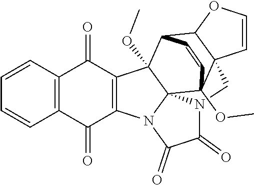 [SMILES: CO[C@@]12C=C[C@H]3C4OC=C[C@]41CN1C(=O)C(=O)N4C5=C(C(=O)c6ccccc6C5=O)[C@]3(OC)[C@]142]

(2a1R,3aS,7S,7aS,16S)-7a,16-Dimethoxy-7,7a-dihydro-3H,8H-6-oxa-2a,13b-diaza-2a1,3a,7-(epiprop[2]ene[1,1,3]triyl)dicyclopenta[cd,g]naphtho[2,3-a]azulene-1,2,8,13(6aH)-tetraone (6h): General procedure G was followed on 200 mg (0.434 mmol) scale of the photoprecursor 4h. After the photochemical reaction afford 168 mg (84%) of product 6h. 1H NMR (500 MHz, CDCl3) δ 8.20-8.14 (m, 1H), 8.10 (dd, J=6.4, 2.6 Hz, 1H), 7.85-7.77 (m, 2H), 6.37 (d, J=2.7 Hz, 1H), 6.32 (d, J=8.9 Hz, 1H), 6.09 (t, J=7.9 Hz, 1H), 5.07 (d, J=3.8 Hz, 1H), 4.89 (d, J=2.8 Hz, 1H), 4.40 (dd, J=6.9, 3.7 Hz, 1H), 4.03 (d, J=11.2 Hz, 1H), 3.52 (s, 3H), 3.42 (d, J=11.3 Hz, 1H), 3.38 (s, 3H); 13C{1H}NMR (126 MHz, CDCl3) δ 182.12, 176.54, 160.08, 159.18, 150.71, 147.17, 134.87, 134.52, 133.89, 132.03, 131.97, 129.98, 127.37, 126.83, 126.67, 98.35, 89.40, 88.72, 84.59, 84.42, 64.24, 55.93, 53.50, 51.88, 37.22. HRMS (ESI) m/z: [M+Na]+ calcd for C25H18N2O7Na 481.1012; found 481.0965.

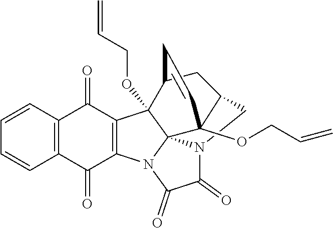 [SMILES: C=CCO[C@@]12C=C[C@H]3C[C@@H]1CN1C(=O)C(=O)N4C5=C(C(=O)c6ccccc6C5=O)[C@]3(OCC=C)[C@]142]

(2R,2aS,2a1R,5R,5aS)-2a,5a-Bis(allyloxy)-2,2a,5,5a-tetrahydro-1H-11b,13a-diaza-2,5-methanobenzo[b]pentaleno[1,6-jk]fluorene-6,11,12,13-tetraone (6i): General procedure G was followed on 200 mg (0.423 mmol) scale of the photoprecursor 4i. After the photochemical reaction afford 150 mg (75%) of product 6i. 1H NMR (500 MHz, CDCl3) δ 8.21-8.14 (m, 1H), 8.11-8.04 (m, 1H), 7.83-7.75 (m, 2H), 6.29-6.19 (m, 2H), 5.89-5.68 (m, 2H), 5.26 (dq, J=17.2, 1.7 Hz, 1H), 5.18 (dt, J=10.5, 1.5 Hz, 1H), 5.14 (dq, J=17.3, 1.8 Hz, 1H), 5.04 (dq, J=10.5, 1.5 Hz, 1H), 4.12 (ddt, J=11.7, 5.1, 1.7 Hz, 3H), 4.03-3.91 (m, 2H), 3.76 (dq, J=6.9, 2.2 Hz, 1H), 3.40 (d, J=11.3 Hz, 1H), 2.60 (ddd, J=11.1, 5.5, 1.8 Hz, 1H), 2.19 (dt, J=13.4, 2.7 Hz, 1H), 1.95 (ddd, J=13.4, 11.0, 2.4 Hz, 1H); 13C{1H}NMR (126 MHz, CDCl3) δ 182.28, 176.57, 160.62, 160.17, 147.12, 134.69, 134.39, 134.37, 133.86, 133.72, 132.08, 132.02, 127.74, 127.33, 126.66, 117.24, 117.02, 88.76, 85.69, 82.81, 67.20, 66.53, 50.12, 39.17, 33.77, 32.67. HRMS (ESI) m/z: [M+Na]+ calcd for C27H22N2O6Na 493.1375; found 493.1373.

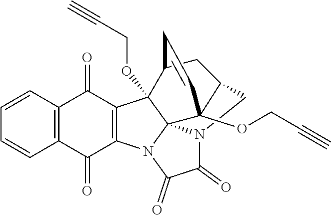 [SMILES: C#CCO[C@@]12C=C[C@H]3C[C@@H]1CN1C(=O)C(=O)N4C5=C(C(=O)c6ccccc6C5=O)[C@]3(OCC#C)[C@]142]

(2R,2aS,2a1R,5R,5aS)-2a,5a-Bis(prop-2-yn-1-yloxy)-2,2a,5,5a-tetrahydro-1H-11b,13a-diaza-2,5-methanobenzo[b]pentaleno[1,6-jk]fluorene-6,11,12,13-tetraone (6j): General procedure G was followed on 200 mg (0.427 mmol) scale of the photoprecursor 4j. After the photochemical reaction afford 172 mg (86%) of product 6j. 1H NMR (500 MHz, CDCl3) δ 8.24-8.17 (m, 1H), 8.17-8.07 (m, 1H), 7.87-7.78 (m, 2H), 6.34 (dd, J=8.9, 1.5 Hz, 1H), 6.30 (dd, J=8.9, 6.8 Hz, 1H), 4.40-4.23 (m, 4H), 4.11 (dd, J=11.3, 5.6 Hz, 1H), 3.83 (ddt, J=6.9, 3.4, 2.1 Hz, 1H), 3.45 (d, J=11.4 Hz, 1H), 2.78 (ddd, J=11.2, 5.5, 1.8 Hz, 1H), 2.51 (t, J=2.4 Hz, 1H), 2.29 (dt, J=15.9, 2.5 Hz, 2H), 2.00 (ddd, J=13.4, 11.1, 2.3 Hz, 1H); 13C{1H}NMR (126 MHz, CDCl3) δ 182.48, 176.45, 160.37, 160.08, 147.35, 134.75, 134.55, 134.42, 133.77, 132.05, 132.01, 127.41, 126.68, 88.75, 86.38, 83.53, 79.90, 79.51, 75.99, 75.87, 54.52, 54.21, 50.07, 39.10, 33.96, 32.62. HRMS (ESI) m/z: [M+Na]+ calcd for C27H18N2O6Na 489.1063; found 489.1039.

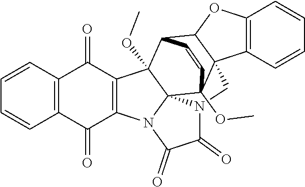 [SMILES: CO[C@@]12C=C[C@H]3C4Oc5ccccc5[C@]41CN1C(=O)C(=O)N4C5=C(C(=O)c6ccccc6C5=O)[C@]3(OC)[C@]142]

(2a1R,3aS,95,9aS,18S)-9a,18-Dimethoxy-9,9a-dihydro-3H,10H-8-oxa-2a,15b-diaza-2a1,3a,9-(epiprop[2]ene[1,1,3]triyl)cyclopenta[cd]indeno[1,2-g]naphtho[2,3-a]azulene-1,2,10,15 (8aH)-tetraone (6k): General procedure G was followed on 200 mg (0.39 mmol) scale of the photoprecursor 4k. After the photochemical reaction afford 180 mg (90%) of product 6k. 1H NMR (500 MHz, CDCl3) δ 8.22-8.15 (m, 1H), 8.15-8.08 (m, 1H), 7.86-7.77 (m, 2H), 7.32 (dd, J=7.5, 1.4 Hz, 1H), 7.24-7.17 (m, 1H), 6.88 (td, J=7.5, 1.0 Hz, 1H), 6.74 (d, J=8.0 Hz, 1H), 6.21-6.15 (m, 1H), 6.15-6.08 (m, 1H), 5.31 (dd, J=4.0, 0.9 Hz, 1H), 4.56-4.50 (m, 1H), 4.32 (d, J=11.4 Hz, 1H), 3.66 (d, J=11.4 Hz, 1H), 3.53 (s, 3H), 3.43 (s, 3H). 13C{1H}NMR (126 MHz, CDCl3) δ 182.07, 176.52, 161.27, 160.22, 159.11, 147.18, 134.93, 134.57, 133.79, 132.04, 131.96, 130.68, 130.56, 127.39, 126.89, 126.07, 125.79, 122.78, 121.05, 109.68, 90.62, 89.25, 84.77, 84.41, 62.19, 56.46, 53.60, 52.35, 37.70. HRMS (ESI) m/z: [M+Na]+ calcd for C29H20N2O7Na 531.1168; found 531.1166.

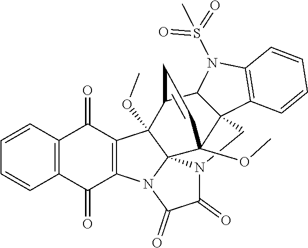 [SMILES: CO[C@@]12C=C[C@H]3C4N(S(C)(=O)=O)c5ccccc5[C@]41CN1C(=O)C(=O)N4C5=C(C(=O)c6ccccc6C5=O)[C@]3(OC)[C@]142]

(2a1R,3aS,9S,9aS,18S)-9a,18-Dimethoxy-9,9a-dihydro-3H,10H-8-amino(methanesulfonamide)-2a,15b-diaza-2a1,3a,9-(epiprop[2]ene[1,1,3]triyl)cyclopenta[cd]indeno[1,2-g]naphtho[2,3-a]azulene-1,2,10,15 (8aH)-tetraone (6l): General procedure G was followed on 200 mg (0.39 mmol) scale of the photoprecursor 41. After the photochemical reaction afford 182 mg (91%) of product 6l. 1H NMR (500 MHz, DMSO-d6) δ 8.11-8.02 (m, 2H), 7.93 (dd, J=5.8, 3.3 Hz, 2H), 7.45 (d, J=7.6 Hz, 1H), 7.36-7.29 (m, 1H), 7.25 (d, J=8.1 Hz, 1H), 7.09 (t, J=7.5 Hz, 1H), 6.32 (dd, J=9.0, 1.4 Hz, 1H), 6.16 (dd, J=9.0, 6.8 Hz, 1H), 4.97 (d, J=3.4 Hz, 1H), 4.46 (dd, J=7.2, 3.5 Hz, 1H), 4.23 (d, J=11.2 Hz, 1H), 4.02 (d, J=11.2 Hz, 1H), 3.50-3.40 (m, 6H), 3.11 (s, 3H); 13C{1H}NMR (126 MHz, DMSO-d6) δ 181.78, 175.86, 160.07, 158.24, 146.69, 143.77, 134.75, 134.52, 133.16, 131.79, 131.60, 130.71, 130.16, 126.80, 126.69, 126.50, 126.30, 126.21, 123.85, 113.23, 88.66, 84.24, 83.87, 70.64, 60.53, 56.18, 53.03, 52.05, 38.03, 34.31.

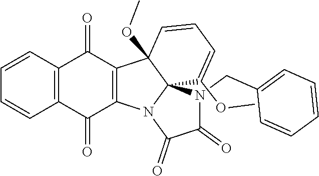 [SMILES: COC1=CC=C[C@@]2(OC)C3=C(C(=O)c4ccccc4C3=O)N3C(=O)C(=O)N(Cc4ccccc4)[C@@]132]

(3aS,7aR)-3-Benzyl-4,7a-dimethoxy-3,7a-dihydro-1H,8H-benzo[b]imidazo[2,1-k]carbazole-1,2,8,13-tetraone (6m): General procedure G was followed on 200 mg (0.425 mmol) scale of the photoprecursor 4m. After the photochemical reaction afford 190 mg (95%) of product 6m. 1H NMR (500 MHz, CDCl3) δ 8.21 (dd, J=6.1, 3.1 Hz, 1H), 8.10 (dd, J=6.0, 3.2 Hz, 1H), 7.84-7.76 (m, 2H), 7.40-7.20 (m, 5H), 6.45 (d, J=10.1 Hz, 1H), 6.06 (dd, J=10.1, 6.5 Hz, 1H), 5.09-5.01 (m, 2H), 4.25 (d, J=15.3 Hz, 1H), 3.35 (s, 3H), 2.96 (s, 3H); 13C{1H}NMR (126 MHz, CDCl3) δ 181.77, 176.69, 158.30, 157.71, 148.89, 145.79, 135.61, 135.31, 134.74, 134.28, 132.31, 131.77, 129.02, 128.18, 127.96, 127.30, 126.85, 122.38, 116.73, 100.19, 84.47, 83.66, 55.18, 53.00, 46.22. HRMS (ESI) m/z: [M+Na]+ calcd for C27H20N2O6Na 491.1219; found 491.1234.

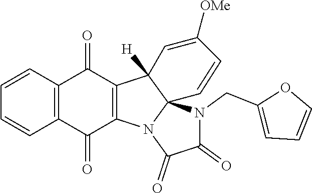 [SMILES: COC1=C[C@H]2C3=C(C(=O)c4ccccc4C3=O)N3C(=O)C(=O)N(Cc4ccco4)[C@]23C=C1]

(3aS,7aS)-3-(Furan-2-ylmethyl)-6-methoxy-3,7a-dihydro-1H,8H-benzo[b]imidazo[2,1-k]carbazole-1,2,8,13-tetraone (6n): General procedure G was followed on 180 mg (0.465 mmol) scale of the photoprecursor 4n. After the reaction afford 135 mg (68%) of product 6n. 1H NMR (500 MHz, CDCl3) δ 8.16 (dd, J=6.0, 3.1 Hz, 1H), 8.03 (dd, J=6.0, 3.2 Hz, 1H), 7.79-7.72 (m, 2H), 7.38 (d, J=1.9 Hz, 1H), 6.32 (dt, J=15.5, 2.9 Hz, 3H), 5.39 (d, J=10.0 Hz, 1H), 5.17 (dd, J=6.5, 2.3 Hz, 1H), 5.09 (d, J=15.6 Hz, 1H), 4.16 (d, J=15.6 Hz, 1H), 4.04 (d, J=6.5 Hz, 1H), 3.63 (s, 3H); 13C{1H}NMR (126 MHz, CDCl3) δ 181.82, 176.29, 157.69, 156.63, 150.54, 147.34, 143.14, 142.92, 137.48, 134.49, 134.23, 132.46, 132.14, 131.67, 127.27, 126.46, 123.66, 110.85, 110.66, 88.91, 81.48, 55.13, 45.82, 37.79. HRMS (ESI) m/z: [M+Na]+ calcd for C24H16N2O6Na 451.0906; found 451.0892.

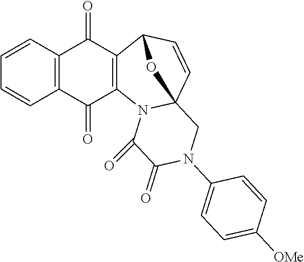 [SMILES: COc1ccc(N2C[C@]34C=C[C@H](O3)C3=C(C(=O)c5ccccc5C3=O)N4C(=O)C2=O)cc1]

(4aS,75)-3-(4-Methoxyphenyl)-3,4-dihydro-7H-4a,7-epoxynaphtho[2,3-f]pyrazino[1,2-a]azepine-1,2,8,13-tetraone (7n): General procedure G was followed with some modifications: DCM (168 mL) was used as a solvent for photo reaction, used 145 mg (0.337 mmol) of the photoprecursor 4n. After the reaction and afford 110 mg (76%) of product 7n. 1H NMR (500 MHz, CDCl3) δ 8.10-8.03 (m, 2H), 7.78-7.71 (m, 2H), 7.34-7.28 (m, 2H), 6.99 (d, J=8.7 Hz, 2H), 6.64-6.59 (m, 1H), 6.39 (d, J=5.8 Hz, 1H), 5.91 (d, J=1.9 Hz, 1H), 4.69 (d, J=12.8 Hz, 1H), 4.00 (d, J=12.9 Hz, 1H), 3.84 (s, 3H); 13C{1H}NMR (126 MHz, CDCl3) δ 181.00, 177.67, 159.21, 154.78, 153.61, 139.79, 134.79, 134.32, 133.99, 133.30, 133.11, 130.71, 129.62, 127.42, 126.38, 126.26, 125.49, 115.03, 95.04, 76.06, 55.73, 53.56. HRMS (ESI) m/z: [M+Na]+ calcd for C24H16N2O6Na 451.0906; found 451.0892.

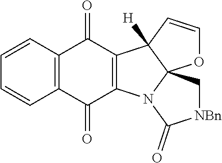 [SMILES: O=C1C2=C(C(=O)c3ccccc31)N1C(=O)N(Cc3ccccc3)C[C@@]13OC=C[C@@H]23]

(3aR,6aS)-2-Benzyl-2,3-dihydro-1H,7H-benzo[f]furo[2,3-b]imidazo[1,5-a]indole-1,7,12(6aH)-trione (6o): General procedure G was followed with some modifications: DCM (168 mL) was used as a solvent for photo reaction, used 130 mg (0.337 mmol) of the photoprecursor 4o. After the reaction afford 101 mg (78%) of product 6o. 1H NMR (500 MHz, CDCl3) δ 8.16 (dd, J=5.9, 3.1 Hz, 1H), 8.07-8.01 (m, 1H), 7.76-7.69 (m, 2H), 7.38-7.27 (m, 5H), 6.39 (t, J=2.5 Hz, 1H), 5.34 (t, J=3.0 Hz, 1H), 4.59 (d, J=14.9 Hz, 1H), 4.43 (d, J=15.0 Hz, 1H), 4.41 (t, J=2.6 Hz, 1H), 3.73 (d, J=10.9 Hz, 1H), 3.62 (d, J=11.0 Hz, 1H); 13C{1H}NMR (126 MHz, CDCl3) δ 182.35, 177.31, 154.83, 146.27, 146.03, 135.43, 134.15, 133.94, 133.79, 132.73, 132.32, 129.10, 128.48, 128.25, 127.42, 125.87, 104.54, 99.57, 55.19, 53.30, 47.90. HRMS (ESI) m/z: [M+Na]+ calcd for C23H16N2O4Na 407.1008; found 407.1014.

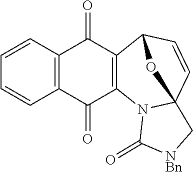 [SMILES: O=C1C2=C(C(=O)c3ccccc31)N1C(=O)N(Cc3ccccc3)C[C@@]13C=C[C@@H]2O3]

(3aS,6S)-2-Benzyl-2,3-dihydro-1H,6H-3a,6-epoxyimidazo[1,5-a]naphtho[2,3-f]azepine-1,7,12-trione (7o): General procedure G was followed on 100 mg (0.26 mmol) scale of the photoprecursor 4o. After the reaction afford 76 mg (76%) of product 70. 1H NMR (500 MHz, CDCl3) δ 8.10-8.08 (m, 1H), 8.06-8.03 (m, 1H), 7.72-7.69 (m, 2H), 7.38-7.34 (m, 2H), 7.33-7.30 (m, 3H), 6.74 (dd, J=5.6, 1.9 Hz, 1H), 5.82 (dd, J=5.6, 0.5 Hz, 1H), 5.70 (d, J=1.9 Hz, 1H), 4.62 (d, J=15.0 Hz, 1H), 4.52 (d, J=15.0 Hz, 1H), 3.86 (d, J=11.3 Hz, 1H), 3.61 (d, J=11.2 Hz, 1H); 13C{1H}NMR (126 MHz, CDCl3) δ 181.86, 179.36, 153.95, 139.43, 138.60, 135.44, 133.83, 133.70, 132.98, 131.10, 130.05, 129.06, 128.33, 128.16, 127.21, 125.97, 123.67, 96.10, 74.92, 49.02, 47.62. HRMS (ESI) m/z: [M+Na]+ calcd for C23H16N2O4Na 407.1008; found 407.1014.

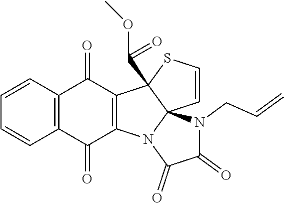 [SMILES: C=CCN1C(=O)C(=O)N2C3=C(C(=O)c4ccccc4C3=O)[C@]3(C(=O)OC)SC=C[C@]123]

Methyl (3aS,13bS)-4-Allyl-5,6,8,13-tetraoxo-5,6,8,13-tetrahydro-4H,13bH-benzo[f]imidazo [1,2-a]thieno[3,2-b]indole-13b-carboxylate (6p): General procedure G was followed on 200 mg (0.473 mmol) scale of the photoprecursor 4p. After the photochemical reaction afford 168 mg (84%) of product 6p. 1H NMR (500 MHz, CDCl3) δ 8.27-8.17 (m, 1H), 8.09 (dd, J=5.9, d, J=6.1 Hz, 1H), 5.36-5.24 (m, 2H), 4.07 (dd, J=15.3, 6.3 Hz, 1H), 3.95 (dd, J=15.3, 6.5 Hz, 1H), 3.75 (s, 3H); 13C{1H}NMR (126 MHz, CDCl3) δ 180.12, 176.38, 164.87, 158.21, 156.04, 144.68, 136.64, 135.63, 134.88, 134.61, 132.11, 131.86, 130.46, 127.56, 126.89, 120.82, 117.84, 99.60, 68.87, 54.79, 45.18; HRMS (ESI) m/z: [M+Na]+ calcd for C21H14N2O6SNa 445.0470; found 445.0472. 3.0 Hz, 1H), 7.88-7.74 (m, 2H), 6.82 (d, J=6.2 Hz, 1H), 5.82 (ddt, J=16.5, 9.6, 6.2 Hz, 1H), 5.44(

### Post Photochemical Modifications

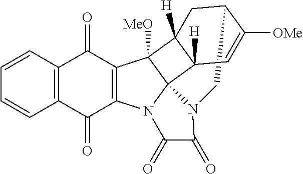 [SMILES: COC1=C[C@H]2[C@H]3C[C@@H]1CN1C(=O)C(=O)N4C5=C(C(=O)c6ccccc6C5=O)[C@]3(OC)[C@]214]

(3aR,3bS,6R,7aR,7bS)-5,7b-Dimethoxy-3b,6,7a,7b-tetrahydro-7H-3,6-methanobenzo[f]benzo[3,4]cyclobuta[1,2-b]imidazo[1,2-a]indole-1,2,8,13-tetraone (8a): Photoproduct 6a (100 mg, 0.239 mmol) was dissolved in DCM (80 mL), and the solution was degassed by bubbling nitrogen or argon for 30 min. The mixture was irradiated with two visible light LEDs, (High Power LED Star #A008-UV410-48; Xmax=410-420 nm), with reaction progress monitored by 1H NMR. DCM was evaporated under a vacuum. The crude residue was dissolved in a minimum amount of DCM followed by the addition of Hexanes, resulted solid was filtered and the solid was washed with 5% DCM in Hexanes solution. The solid was dried under vacuum to offer 70 mg (70%) of the photo-rearranged product 8a. 1H NMR (500 MHz, CDCl3) δ 8.25-8.19 (m, 1H), 8.16 (dt, J=7.0, 3.3 Hz, 1H), 7.83 (dq, J=7.8, 3.9 Hz, 2H), 4.52 (d, J=7.2 Hz, 1H), 4.24 (dd, J=13.2, 4.5 Hz, 1H), 3.59-3.51 (m, 4H), 3.32-3.20 (m, 5H), 2.90 (dq, J=4.9, 2.5 Hz, 1H), 2.44 (dd, J=14.2, 2.5 Hz, 1H), 1.93 (ddd, J=13.5, 7.8, 4.5 Hz, 1H); 13C{1H}NMR (126 MHz, CDCl3) δ 182.41, 177.06, 162.81, 157.51, 156.81, 146.57, 136.06, 134.93, 134.41, 132.49, 132.15, 127.48, 126.94, 86.18, 84.86, 82.65, 55.23, 54.49, 43.81, 39.82, 38.07, 37.31, 24.97. HRMS (ESI) m/z: [M+Na]+ calcd for C23H18N2O6Na 441.1063; found 441.1059.

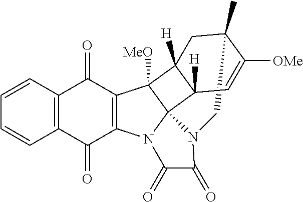 [SMILES: COC1=C[C@H]2[C@H]3C[C@]1(C)CN1C(=O)C(=O)N4C5=C(C(=O)c6ccccc6C5=O)[C@]3(OC)[C@]214]

(3aR,3bS,6R,7aR,7bS)-5,7b-Dimethoxy-6-methyl-3b,6,7a,7b-tetrahydro-7H-3,6-methanobenzo[f]benzo[3,4]cyclobuta[1,2-b]imidazo[1,2-alindole-1,2,8,13-tetraone (8c): Photoproduct 6c (100 mg, 0.231 mmol) was dissolved in DCM (80 mL), and the solution was degassed by bubbling nitrogen or argon for 30 min. The mixture was irradiated with two visible light LEDs, (High Power LED Star #A008-UV410-48; λmax=410-420 nm), with reaction progress monitored by 1H NMR. DCM was evaporated under a vacuum. The crude residue was dissolved in a minimum amount of Methanol, resulted solid was filtered and solid was washed with pre-cooled Methanol. The solid was dried under vacuum to offer 65 mg (65%) of the photo-rearranged product 8c. 1H NMR (500 MHz, CDCl3) δ 8.25-8.20 (m, 1H), 8.20-8.14 (m, 1H), 7.83 (p, J=7.5 Hz, 2H), 4.52-4.47 (m, 1H), 4.03 (d, J=13.1 Hz, 1H), 3.53 (s, 3H), 3.35 (d, J=13.2 Hz, 1H), 3.29-3.20 (m, 5H), 2.30 (d, J=14.2 Hz, 1H), 1.79 (dd, J=14.4, 7.3 Hz, 1H), 1.31 (s, 3H); 13C{1H}NMR (126 MHz, CDCl3) δ 182.42, 177.04, 163.61, 157.28, 156.57, 146.57, 136.14, 134.94, 134.41, 132.49, 132.13, 127.48, 126.93, 85.91, 84.47, 82.63, 55.30, 54.49, 49.58, 39.94, 39.48, 38.07, 34.02, 21.60. HRMS (ESI) m/z: [M+Na]+ calcd for C₂₄H20N2O6Na 455.1219; found 455.1213.

### Thermal Reaction of Photoproduct 4 (General Procedure H):

An oven-dried heavy-wall glass tube was charged with photoproduct (0.12 mmol) in DMSO (8 mL) and stirred at 160° C. for 36 to 48 h. After completion of the reaction (indicated by 1H NMR), the reaction mixture was concentrated and separated by flash chromatography on silica gel.

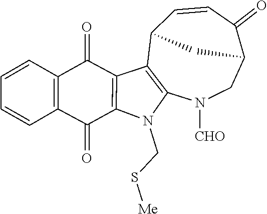 [SMILES: CSCn1c2c(c3c1N(C=O)C[C@H]1C[C@@H]3C=CC1=O)C(=O)c1ccccc1C2=O]

(3R,7R)-14-((Methylthio)methyl)-4,8,13-trioxo-3,4,7,8,13,14-hexahydro-3,7-methanoazonino[2,3-b]benzo[f]indole-1(2H)-carbaldehyde (9a): General procedure H was followed on 50 mg (0.119 mmol) scale of the photoproduct 6a. After the reaction, the crude product was purified by flash chromatography on silica gel (75% ethyl acetate in hexanes) to afford 32 mg (66%) of the title compound 9a. 1H NMR (500 MHz, CDCl₃) δ 8.16 (tq, J=7.3, 3.3 Hz, 2H), 7.99 (s, 1H), 7.72 (dp, J=7.4, 4.4 Hz, 2H), 7.02 (m, 1H), 6.46 (d, J=14.5 Hz, 1H), 6.07 (d, J=9.9 Hz, 1H), 5.10 (t, J=5.6 Hz, 1H), 4.76 (d, J=14.4 Hz, 1H), 4.35 (dd, J=14.4, 4.2 Hz, 1H), 3.57 (dd, J=14.3, 1.9 Hz, 1H), 2.89 (d, J=5.4 Hz, 1H), 2.70 (dt, J=13.5, 5.6 Hz, 1H), 2.15 (q, J=2.8 Hz, 1H), 2.11 (s, 3H); 13C {1H}NMR (126 MHz, CDCl₃) δ 200.10, 182.19, 176.14, 161.99, 149.86, 136.07, 133.77, 133.65, 133.63, 133.53, 128.74, 128.59, 126.86, 126.56, 122.90, 118.31, 53.28, 48.33, 45.73, 30.63, 28.46, 15.04. HRMS (ESI) m/z: [M+Na]⁺ calcd for C22H16N2O4SNa 429.0885; found 429.0890.

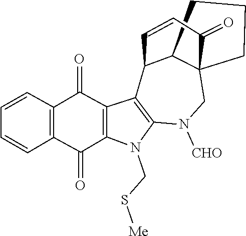

(3aR,13S,13aR)-6-((Methylthio)methyl)-7,12,16-trioxo-1,2,3,6,7,12,13,13a-octahydro-13,3a-prop[1]enobenzo[f]cyclopenta[5,6]azepino[2,3-b]indole-5(4H)-carbaldehyde (9g): General procedure H was followed on 50 mg (0.109 mmol) scale of the photoproduct 6g. After the reaction, the crude product was purified by flash chromatography on silica gel (75% ethyl acetate in hexanes) to afford 39 mg (80%) of the title compound 9g. ¹H NMR (500 MHz, CDCl₃) δ 8.21-8.12 (m, 2H), 7.93 (s, 1H), 7.77-7.68 (m, 2H), 6.89 (m, 1H), 6.46 (d, J=14.3 Hz, 1H), 6.04 (d, J=9.9 Hz, 1H), 5.07 (d, J=6.0 Hz, 1H), 4.74 (d, J=14.4 Hz, 1H), 4.26 (d, J=13.9 Hz, 1H), 3.28 (d, J=14.0 Hz, 1H), 2.54 (m, 1H), 2.36-2.23 (m, 1H), 2.13 (q, J=5.6 Hz, 1H), 2.10 (s, 3H), 1.81-1.54 (m, 3H), 1.46-1.36 (m, 1H); ¹³C {¹H}NMR (126 MHz, CDCl₃) δ 202.75, 182.18, 176.13, 162.17, 147.16, 135.29, 133.83, 133.63, 133.56, 128.34, 127.73, 126.88, 126.55, 122.69, 119.24, 58.38, 57.46, 48.18, 48.09, 33.70, 31.80, 30.26, 20.70, 15.02. HRMS (ESI) m/z: [M+Na]⁺ calcd for C₂₅H₂₂N₂O₄SNa 469.1198; found 469.1194.

### Hetero Diels-Alder Cycloaddition (General Procedure I)

1 eq of photoproduct and 1 eq of 1,3-dicarbonyl compound were dissolved in 0.7 mL of dry acetonitrile. To this was added 0.08 eq of L-proline and 1.3 eq of 37% aq. formaldehyde solution. The reaction stirred at 70° C. until full consumption of the photoproduct, as determined by ¹H NMR. The reaction was diluted with water and extracted with EtOAc. The organic layer was separated, dried over Na₂SO₄, and concentrated in vacuo. The mixture was then purified by flash chromatography.

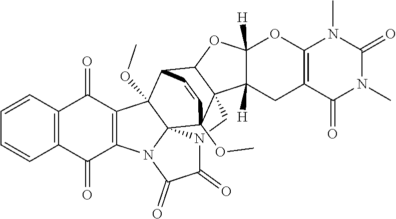

(2a1R,8bS,9S,10aS,16aS,16bS,18S)-8b,18-Dimethoxy-12,14-dimethyl-9,9a,10a,12,16,16a-hexahydro-8H,13H,17H-10,11-dioxa-2a,12,14,17a-tetraaza-2a1,16b,9-(epiprop[2]ene[1,1,3]triyl)benzo[5,6]indeno[1,2-g]cyclopenta[cd]naphtho[2,3-a]azulene-1,2,3,8,13,15(8bH,14H)-hexaone (11h): General procedure I was followed, from 62.0 mg of 6h (0.135 mmol) and 21 mg of 1,3-dimethyl barbituric acid (0.135 mmol) was obtained 70 mg (83%) of the title compound 11h. 1H NMR (500 MHz, CDCl₃) δ 8.21-8.16 (m, 1H), 8.13-8.08 (m, 1H), 7.85-7.80 (m, 2H), 6.36 (d, J=8.9 Hz, 1H), 6.27 (t, J=7.9 Hz, 1H), 5.67 (d, J=3.7 Hz, 1H), 4.75 (d, J=3.3 Hz, 1H), 4.30-4.25 (m, 1H), 3.84 (d, J=11.1 Hz, 1H), 3.55 (d, J=1.6 Hz, 3H), 3.40-3.34 (m, 9H), 3.18 (d, J=11.0 Hz, 1H), 2.86 (d, J=17.8 Hz, 1H), 2.69 (dd, J=17.9, 6.8 Hz, 1H), 2.51 (d, J=6.2 Hz, 1H); ¹³C {¹H}NMR (126 MHz, CDCl₃) δ 182.14, 176.47, 162.26, 159.52, 159.33, 154.22, 151.04, 147.01, 134.98, 134.70, 133.92, 132.59, 131.96, 127.46, 126.89, 125.50, 104.45, 89.55, 89.05, 85.91, 84.69, 83.53, 58.12, 56.29, 53.48, 49.57, 39.98, 37.26, 28.87, 28.27, 18.12. HRMS (ESI) m/z: [M+Na]⁺ calcd for C₃₂H₂₆N₄O₁₀Na 649.1547; found 649.1570.

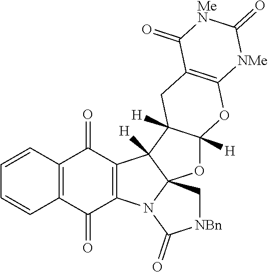 [SMILES: Cn1c2c(c(=O)n(C)c1=O)C[C@@H]1[C@H](O2)O[C@]23CN(Cc4ccccc4)C(=O)N2C2=C(C(=O)c4ccccc4C2=O)[C@H]13]

(10bS,10cR,16aS,17aR)-2-Benzyl-13,15-dimethyl-1,2,10c,11,15,16a-hexahydro-3H,10H,12H-benzo[f]imidazo[1,5-a]pyrimido[5″,4″:5′,6′]pyrano[3′,2′:4,5]furo[2,3-b]indole-3,5,10,12,14 (10bH,13H)-pentaone (110): General procedure I was followed, from 50.0 mg of 6o (0.13 mmol) and 20.3 mg of 1,3-dimethyl barbituric acid (0.13 mmol) was obtained 31 mg (43%) of the title compound 11o. ¹H NMR (500 MHz, CDCl₃) δ 8.19-8.12 (m, 1H), 8.08-8.01 (m, 1H), 7.79-7.72 (m, 2H), 7.40-7.32 (m, 3H), 7.30-7.25 (m, 2H), 5.81 (d, J=3.5 Hz, 1H), 4.60 (d, J=14.9 Hz, 1H), 4.39 (d, J=14.8 Hz, 1H), 3.66-3.57 (m, 3H), 3.34 (s, 3H), 3.29 (s, 3H), 2.97-2.84 (m, 3H); ¹³C {¹H}NMR (126 MHz, CDCl₃) δ 182.09, 176.91, 162.59, 154.27, 153.79, 150.90, 146.22, 135.03, 134.25, 134.01, 132.56, 132.15, 131.93, 129.16, 128.48, 128.37, 127.45, 126.07, 102.70, 101.71, 84.21, 54.88, 53.86, 47.87, 40.96, 28.83, 28.27, 19.39. HRMS (ESI) m/z: [M+Na]⁺ calcd for C₃₀H₂₄N₄O₇Na 575.1543; found 575.1555.

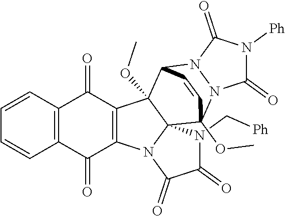

(3aS,4R,10R,10aR)-3-Benzyl-4,10a-Dimethoxy-7-phenyl-10,10a-dihydro-1H,4H,6H-4,10-etheno[1,2,4]triazolo[1′,2′:1,2]pyridazino[4,5-b]benzo[f]imidazo[1,2-a]indole-1,2,6,8,11,16(3H,7H)-hexaone (10m): To a stirred solution of the photoproduct 6m (47 mg, 0.1 mmol) in DCM (4 mL) was added 4-phenyl-3H-1,2,4-triazole-3,5(4H)-dione (19.3 mg, 0.11 mmol). The resulting mixture was stirred at room temperature for 1 h. After completion of the reaction (progress of the reaction was monitored by ¹H NMR). Diluted reaction mixture with Hexanes and filtered, washed solid with Hexanes to give 62 mg (97%) of the title compound 10m. ¹H NMR (500 MHz, CDCl₃) δ 8.16 (d, J=7.4 Hz, 2H), 7.83-7.73 (m, 2H), 7.41-7.31 (m, 5H), 7.31-7.21 (m, 3H), 7.18-7.11 (m, 2H), 6.89 (dd, J=8.7, 6.0 Hz, 1H), 6.15 (dd, J=8.7, 1.5 Hz, 1H), 5.80 (dd, J=5.9, 1.4 Hz, 1H), 5.12 (d, J=15.6 Hz, 1H), 4.68 (d, J=15.6 Hz, 1H), 3.49 (s, 3H), 3.28 (s, 3H); ¹³C {¹H}NMR (126 MHz, CDCl₃) δ 181.12, 175.69, 158.70, 157.85, 152.42, 151.78, 146.40, 135.39, 135.03, 134.50, 132.16, 132.12, 130.72, 129.54, 129.08, 128.84, 128.78, 128.46, 128.19, 127.59, 127.09, 125.02, 91.80, 89.44, 86.22, 56.25, 55.68, 52.60, 47.84, 29.88. HRMS (ESI) m/z: [M+Na]⁺ calcd for C₃₅H₂₅N₅O₈Na 666.1601; found 666.1584.

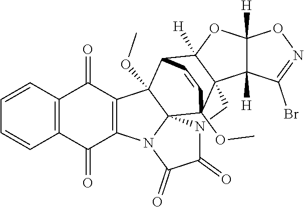

(2a1R,3aS,3bR,6aR,7aR,8S,8aS,17S)-4-Bromo-8a,17-dimethoxy-3b,6a,8,8a-tetrahydro-3H,9H-6,7-dioxa-2a,5,14b-triaza-2a1,3a,8-(epiprop[2]ene[1,1,3]triyl)cyclopenta[cd]naphtho[2,3-a]pentaleno[1,2-g]azulene-1,2,9,14(7aH)-tetraone (12h): To a stirred solution of the photoproduct 6h (100 mg, 0.218 mmol) in DCM (4 mL) was added dibromoformaldoxime (133 mg, 0.654 mmol) and Na₂CO₃ (139 mg, 1.308 mmol) at room temperature. The resulting mixture was stirred at room temperature for 3 days. After completion of the reaction (progress of the reaction was monitored by 1H NMR), water (10 mL) was added, and organics were extracted with DCM (3×10 mL). Combined organic extracts were dried over anhydrous Na₂SO₄ and concentrated under vacuum to give crude product which was purified by flash chromatography on silica gel to give 98 mg (77%) of the title compound 12h. ¹H NMR (500 MHz, CDCl3) δ 8.21-8.16 (m, 1H), 8.14-8.07 (m, 1H), 7.85-7.80 (m, 2H), 6.35 (dd, J=8.9, 1.5 Hz, 1H), 6.27 (dd, J=8.9, 6.8 Hz, 1H), 6.05 (d, J=5.9 Hz, 1H), 4.87 (d, J=3.9 Hz, 1H), 4.69 (d, J=11.4 Hz, 1H), 4.36-4.32 (m, 1H), 3.75 (d, J=5.8 Hz, 1H), 3.63 (s, 3H), 3.51 (d, J=11.5 Hz, 1H), 3.37 (s, 3H); ¹³_{C}{¹H}NMR (126 MHz, CDCl₃) δ 182.02, 176.53, 159.49, 159.18, 147.02, 137.94, 135.04, 134.70, 134.35, 133.84, 131.97, 131.92, 127.43, 126.95, 124.08, 111.36, 88.93, 88.59, 83.83, 83.75, 62.90, 58.18, 56.78, 53.59, 47.76, 38.76. HRMS (ESI) m/z: [M+Na]⁺ calcd for C₂₆H₁₈BrN₃O₈Na 604.0159; found 604.0107.

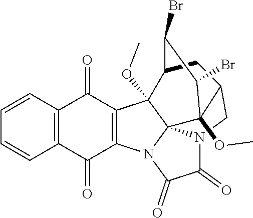 [SMILES: CO[C@]12C3=C(C(=O)c4ccccc4C3=O)N3C(=O)C(=O)N4CC5C[C@@H]1[C@@H](Br)[C@H](Br)[C@@]5(OC)[C@@]432]

(2aS,2a1S,3R,4R,5S)-3,4-Dibromo-2a,5a-dimethoxy-2,2a,3,4,5,5a-hexahydro-1H-11b,13a-diaza-2,5-methanobenzo[b]pentaleno[1,6-jk]fluorene-6,11,12,13-tetraone (13a): A solution of Br₂ (24 mg, 0.15 mmol) in CHCl₃ (1 mL) was slowly added to a solution of photo-product 6a (42 mg, 0.1 mmol) in CHCl₃ (4 mL). The reaction mixture was heated to 40° C. for overnight. After completion of the reaction (progress of the reaction was monitored by 1H NMR) cooled to rt. The reaction was quenched with a sat. solution of NaHSO₃ (5 mL). The layers were separated, and the aqueous layer was extracted with CH₂Cl₂ (10 mL). The combined organic layers were washed with brine (10 mL) and dried over Na₂SO₄. Filtration, evaporation of the solvent, and further purification by flash chromatography on silica gel (pentane) gave 52 mg (90%) of the title compound 13a. 1H NMR (500 MHz, CDCl₃) δ 8.25-8.19 (m, 1H), 8.19-8.13 (m, 1H), 7.89-7.80 (m, 2H), 4.54 (d, J=3.8 Hz, 1H), 4.40-4.34 (m, 1H), 3.87 (dd, J=11.2, 5.3 Hz, 1H), 3.35-3.26 (m, 4H), 3.22 (s, 3H), 3.16-3.11 (m, 1H), 2.98-2.84 (m, 2H), 2.15-2.09 (m, 1H); ¹³C {¹H}NMR (126 MHz, CDCl₃) δ 182.98, 175.99, 160.83, 159.22, 149.30, 134.95, 134.74, 132.05, 131.95, 127.69, 127.06, 87.60, 84.68, 78.17, 53.59, 53.56, 51.09, 50.07, 49.92, 37.54, 34.83, 27.39. HRMS (ESI) m/z: [M+Na]⁺ calcd for C₂₃H₁₈Br₂N₂O₆Na 600.9410; found 600.9362.

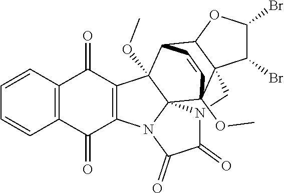

(2a1S,3aR,4R,5R,7S,7aS,16S)-4,5-Dibromo-7a,16-dimethoxy-4,5,7,7a-tetrahydro-3H,8H-6-oxa-2a,13b-diaza-2a1,3a,7-(epiprop[2]ene[1,1,3]triyl)dicyclopenta[cd,g]naphtho[2,3-a]azulene-1,2,8,13(6aH)-tetraone (13h): A solution of Br₂ (24 mg, 0.15 mmol) in CHCl₃ (1 mL) was slowly added to a solution of photoproduct 6h (46 mg, 0.1 mmol) in CHCl₃ (4 mL). The reaction mixture was stirred at room temperature. After completion of the reaction (progress of the reaction was monitored by 1H NMR) cooled to rt. The reaction was quenched with a sat. solution of NaHSO₃ (5 mL). The layers were separated, and the aqueous layer was extracted with CH₂Cl₂ (10 mL). The combined organic layers were washed with brine (10 mL) and dried over Na₂SO₄. Filtration, evaporation of the solvent, and further purification by flash chromatography on silica gel (Hexanes) gave 52 mg (84%) of the title compound 13h. ¹H NMR (500 MHz, CDCl₃) δ 8.21-8.15 (m, 1H), 8.12-8.07 (m, 1H), 7.86-7.79 (m, 2H), 6.51 (d, J=4.2 Hz, 1H), 6.35 (d, J=8.9 Hz, 1H), 6.26 (dd, J=9.0, 6.6 Hz, 1H), 4.99 (d, J=3.6 Hz, 1H), 4.51 (d, J=11.5 Hz, 1H), 4.33-4.27 (m, 1H), 4.20 (d, J=4.1 Hz, 1H), 3.80 (d, J=11.5 Hz, 1H), 3.55 (s, 3H), 3.39 (s, 3H); ¹³C {¹H}NMR (126 MHz, CDCl₃) δ 182.05, 176.41, 159.72, 159.09, 147.10, 135.01, 134.70, 133.57, 133.39, 131.93, 127.46, 126.89, 125.05, 93.76, 89.25, 89.12, 85.54, 84.22, 57.98, 56.31, 53.61, 52.03, 50.25, 36.85. HRMS (ESI) m/z: [M+Na]⁺ calcd for C₂₅H₁₈Br₂N₂O₇Na 638.9379, found 638.9478.

### Photochemical Reactions and Post-Photochemical Modifications

### Flow Reactor Setup

Large-scale photoirradiations were carried out using a flow reactor. A 100-mL syringe containing the photoprecursor solution was placed into a syringe pump. The syringe was equipped with a 14 Ga needle (50 mm length) interfaced to PFTE tubing (1.5 mm ID, 2.1 mm OD). The tubing was coiled around a glass rod and secured in place with epoxy glue. To prevent thermal degradation of the photoproducts due to heat generated by LED lamps, the glass rod with tubing was inserted into a reflux condenser connected to tap water flow. The condenser was covered with aluminum foil, leaving a 10 mm-wide window for irradiation (the internal volume exposed to irradiation is ca. 0.6 mL). An irradiator containing two visible light LEDs (High Power LED Star #A008-UV410-48; [symbol] max=410-420 nm; board configuration: 3-Up A008) installed on a heat sink with fan and driven by a RACD30-700 power supply (output: 700 mA, 10-43 VDC), was placed ca. 2-5 mm away from the irradiation window. A second end of the tubing was inserted into a rubber septum placed on top of the receiving flask. To release the pressure building up in the system during the experiment, a needle was injected into the same septum. The syringe and the receiving flask were covered with aluminum foil in order to prevent overirradiation of the starting material and the photoproducts.

### Effect of the Flow Rate on the Product Yield

5 mM solution of photoprecursor (0.064 g, 0.173 mmol) in DCM (34.5 mL) was degassed by bubbling argon for 40 min. 3.5 mL portions were run through the flow reactor at various flow rates. The resulting solutions were collected in 1-mL syringes (to measure volume), evaporated, and residues were redissolved in 0.50 mL of 0.012 M solution of 1,4-dimethoxybenzene (internal standard) in CDCl3 to determine yields by 1H NMR (if necessary, the yield was corrected to account for the difference in volumes of collected solutions).

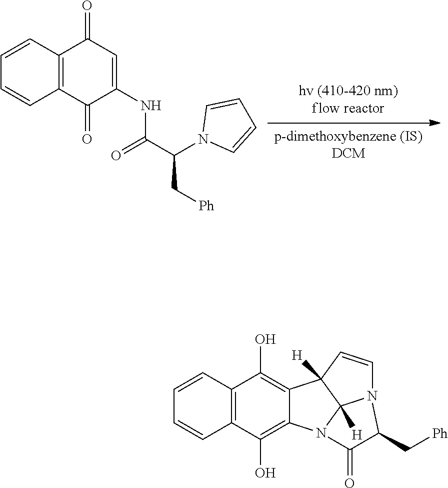

| Flow | Volume | Correction | ¹H NMR |
|---|---|---|---|
| rate (mL/h) | collected (mL) | coefficient | yield (%) |
| 6 (no | 0.60 | — | — |
| irradiation) | | | |
| 4 | 0.58 | 0.60/0.58 | 33 |
| 6 | 0.60 | 0.60/0.60 | 41 |
| 9 | 0.60 | 0.60/0.60 | 41 |
| 13.5 | 0.50 | 0.60/0.50 | 37 |
| 18 | 0.58 | 0.60/0.58 | 37 |

### General Procedure J1 (Photochemical Reaction)

5 mM solution of photo precursor was degassed by bubbling argon for 30-60 min, transferred to a 100-mL syringe under argon, and then irradiated using the photochemical flow reactor setup described above. Initial flow rate was estimated using the time it took for an NMR-scale photoreaction (ca. 0.5 mL of a solution) to come to completion:

$$Flow rate [ mL / h ] = 0.5 / NMR - scale time [ min ] \times 60$$

After ca. 0.5 mL of the irradiated solution was collected in the receiving flask, the solvent was removed in vacuo, the residue was redissolved in a deuterated solvent and checked by 1H NMR. If the spectrum revealed more than 15 mol % of the starting material in the mixture, the flow rate was decreased by 2 mL/h, and the irradiated solution was tested by 1H NMR again in the same manner to ensure adequate purity of the photoproduct.

With the optimized flow rate, all remaining photoprecursor was irradiated. The resulting solution of photoproduct was either used directly for post-photochemical steps (in case of DCM) or evaporated prior to further modifications (in case of DMSO).

### General Procedure J2 (Post-Photochemical Oxidation of Hydroquinone)

Solution of hydroquinone photoproduct (0.190 mmol) in DCM was treated with MnO2 (1.90 mmol) and stirred at ambient temperature for 1 h. The mixture was then filtered through celite (cake washed with 75 mL of DCM). The filtrate was evaporated, and the residue was redissolved in ethyl acetate (50 mL). The solution was washed with water (6×10 mL) followed by brine (10 mL), dried over anhydrous Na2SO4, filtered, and the solvent was removed in vacuo to yield the oxidized photoproduct (if necessary, it was additionally purified by flash chromatography on silica gel).

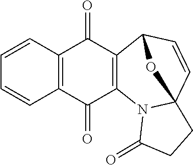 [SMILES: O=C1C2=C(C(=O)c3ccccc31)N1C(=O)CC[C@@]13C=C[C@@H]2O3]

(1R,16S)-19-Oxa-5-azapentacyclo[14.2.1.01,5.06,15.08,13]nonadeca-6(15),8(13),9,11,17-pentaene-4,7,14-trione (7r): General Procedure J1 was followed on a 0.185 mmol scale of 4r (solvent=DMSO; flow rate=8 mL/h). The primary photoproduct was then oxidized according to General Procedure J2 (in 2.7 mL of DCM), which afforded 0.046 g (85% after two steps) of the title compound 7r (flash chromatography was not required). 1H NMR (500 MHz, CDCl3) δ 8.09-8.06 (m, 1H), 8.06-8.03 (m, 1H), 7.75-7.69 (m, 2H), 6.73 (dd, J=5.6, 1.9 Hz, 1H), 5.87 (d, J=5.6 Hz, 1H), 5.71 (d, J=1.9 Hz, 1H), 2.89-2.79 (m, 1H), 2.77-2.67 (m, 2H), 2.56-2.47 (m, 1H). 13C NMR (126 MHz, CDCl3) δ 182.1, 178.9, 173.0, 138.9, 137.7, 134.0, 133.8, 132.9, 131.2, 131.0, 127.2, 126.5, 126.1, 101.3, 75.6, 29.3, 26.6. HRMS (ESI) m/z: [M+H]+ calcd for C17H12NO4 294.0761, found 294.0765.

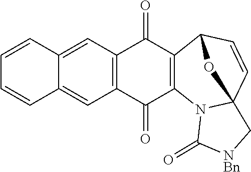 [SMILES: O=C1C2=C(C(=O)c3cc4ccccc4cc31)N1C(=O)N(Cc3ccccc3)C[C@@]13C=C[C@@H]2O3]

(1S,20S)-3-Benzyl-23-oxa-3,5-diazahexacyclo[18.2.1.01,5.06,19.08,17.010,15]tricosa-6(19),8(17),9,11,13,15,21-heptaene-4,7,18-trione (7q): General Procedure J1 was followed on a 0.229 mmol scale of 57452 (solvent=DMSO; flow rate=13 mL/h). The primary photoproduct was then oxidized according to General Procedure J2 (in 3.4 mL of DCM), which after flash chromatography on silica gel (eluent: chloroform) afforded 0.047 g (47% after two steps) of the title compound. 1H NMR (500 MHz, CDCl3) δ 8.63 (s, 1H), 8.59 (s, 1H), 8.08-8.00 (m, 2H), 7.71-7.65 (m, 2H), 7.42-7.28 (m, 5H), 6.79 (dd, J=5.6, 1.9 Hz, 1H), 5.85 (d, J=5.7 Hz, 1H), 5.77 (d, J=1.8 Hz, 1H), 4.65 (d, J=15.0 Hz, 1H), 4.53 (d, J=15.0 Hz, 1H), 3.88 (d, J=11.2 Hz, 1H), 3.63 (d, J=11.2 Hz, 1H). 13C NMR (126 MHz, CD2Cl2) δ 181.7, 179.3, 154.4, 140.2, 139.9, 136.3, 135.3, 135.2, 132.3, 130.6, 130.6, 130.0, 130.0, 129.8, 129.4, 129.4, 128.6, 128.5, 128.4, 128.4, 124.2, 96.6, 75.6, 49.5, 47.8. HRMS (ESI) m/z: [M+Na]+ calcd for C27H18N2O4Na 457.1164, found 457.1160.

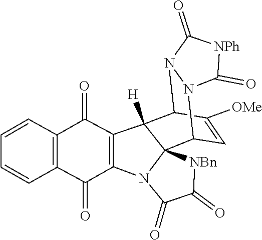

(2R,17R)-3-Benzyl-25-methoxy-21-phenyl-3,6,19,21,23 pentaazaheptacyclo[16.5.2.02,6.02,17.07,16.09,14.019,23]pentacosa-7(16),9(14),10,12,24-pentaene-4,5,8,15,20,22-hexone (10s): General Procedure J was followed on a 0.365 mmol scale of 1s (solvent=DMSO; flow rate=7 mL/h). The primary photoproduct was then redissolved in DCM (5.5 mL) and treated with MnO2 (0.318 g, 3.65 mmol). After 1 h stirring at ambient temperature, 4-phenyl-1,2,4-triazoline-3,5-dione (0.064 g, 0.37 mmol) was added, and the mixture was stirred for additional 5 h. The product was then subjected to work-up, as described in General Procedure C, and then purified by recrystallization as follows. Crude product was dissolved in ethyl acetate (4 mL). Hexane (1 mL) was added, after which some precipitation was observed. Additional portion of ethyl acetate (1 mL) was added to dissolve precipitate. The solution was kept for crystallization at room temperature. After 2 days, ca. ¾ of liquid evaporated, and dark yellow solid formed. Mother liquor was removed by decantation; the solid was washed with hexane-ethyl acetate 1:1 (3×1 mL) followed by ether (3×1 mL) and dried in vacuo, which afforded 0.128 g (57% after three steps) of the title compound. 1H NMR (500 MHz, CDCl3) δ 8.25-8.15 (m, 1H), 8.14-8.04 (m, 1H), 7.84-7.72 (m, 2H), 7.42-7.28 (m, 8H), 7.14 (d, J=7.4 Hz, 2H), 5.52 (t, J=3.2 Hz, 1H), 4.98 (d, J=6.4 Hz, 1H), 4.90 (d, J=15.7 Hz, 1H), 4.82 (d, J=15.6 Hz, 1H), 4.77 (dd, J=6.5, 2.8 Hz, 1H), 3.83 (d, J=3.8 Hz, 1H), 3.67 (s, 3H). 13C NMR (126 MHz, CDCl3) δ 181.5, 176.0, 158.6, 158.5, 157.2, 156.5, 155.9, 145.2, 134.6, 134.5, 134.5, 132.7, 132.3, 131.9, 130.8, 129.6, 129.2, 128.7, 128.7, 127.5, 126.7, 126.7, 125.3, 89.6, 84.7, 61.5, 56.4, 54.6, 47.3, 46.4. HRMS (ESI) m/z: [M+H]+ calcd for C34H24N5O7 614.1670, found 614.1680.

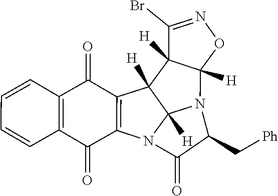

(2S,2a1R,2bS,5aS,5bS)-2-benzyl-5-bromo-2a1,2b,5a,5b-tetrahydro-3-oxa-2a,4,11b-triazapentaleno[1′,2′,3′:3,4]pentaleno[1,2-b]naphthalene-1,6,11(2H)-trione (12r): 5 mM solution of photoprecursor 1r (0.112 g, 0.302 mmol) was dissolved in anhydrous DCM (61 mL), and the solution was degassed by bubbling argon for 1 h. Under Ar, transferred to a 100-mL plastic syringe and irradiated using flow reactor with LED-412 (two 2.9 W@412 nm LED Engin chips) at 8 mL/h with reaction progress monitored by 1H NMR. Evaporated reaction solvent then re-dissolved residue in DCM (6 mL) followed by the addition of dibromoformaldoxime (0.307 g, 1.51 mmol) and potassium bicarbonate (0.302 mg, 3.02 mmol). The mixture was stirred at ambient temperature for 24 hours. The organic layer was washed with water (3×5 mL) and brine (2×5 mL), dried over anhydrous Na₂SO₄, and the solvent was removed in vacuo. Further purification by flash chromatography on silica gel (gradient elution: Hexanes-Ethyl acetate+1% TEA) afforded 36 mg (24% after two steps) of 12r as solid. 1H NMR (500 MHz, CDCl3) δ 8.11-8.05 (m, 1H), 8.03-7.96 (m, 1H), 7.72-7.65 (m, 2H), 7.31-7.16 (m, 5H), 5.81 (d, J=5.9 Hz, 1H), 5.72 (d, J=8.1 Hz, 1H), 4.30 (dd, J=9.5, 4.3 Hz, 1H), 4.03 (dd, J=10.1, 8.0 Hz, 1H), 3.92 (dd, J=10.1, 5.9 Hz, 1H), 3.16 (dd, J=14.3, 4.4 Hz, 1H), 3.06 (dd, J=14.3, 9.5 Hz, 1H); 13C{1H} NMR (126 MHz, Acetone-d6) δ 182.27, 176.80, 174.53, 145.93, 139.55, 137.03, 134.39, 134.00, 132.81, 132.34, 129.36, 128.86, 127.47, 127.34, 126.30, 106.47, 89.92, 69.03, 60.70, 44.14, 35.79. HRMS (ESI) m/z: [M+H]+ calcd for C24H17BrN3O4 490.0397; found 490.0403.

Proton NMR, Carbon ¹³NMR, and mass spec data for compounds synthesized are listed in Table 5.

TABLE 5
Spectroscopic Data for Synthesized compounds

| Compound | | |
|---|---|---|
| No. | Chemical Structure | Spectroscopic Data |
| 4a | 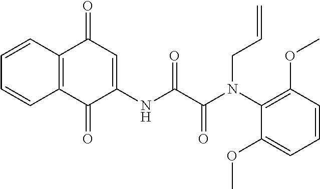 [SMILES: C=CCN(C(=O)C(=O)NC1=CC(=O)c2ccccc2C1=O)c1c(OC)cccc1OC] | ¹H NMR (500 MHz, CDCl₃) δ 9.86 (s, 1H), 8.15-8.10 (m, 1H), 8.05 (d, J = 7.8 Hz, 1H), 7.78-7.69 (m, 2H), 7.65 (s, 1H), 7.28-7.23 (m, 1H), 6.59 (d, J = 8.3 Hz, 2H), 5.86 (ddt, J = 16.8, 9.6, 6.6 Hz, 1H), 5.15-5.04 (m, 2H), 4.31 (d, J = 6.6 Hz, 2H), 3.80 (s, 6H); ¹³C{¹H} NMR (126 MHz, CDCl₃) δ 185.18, 180.46, 161.85, 159.32, 155.67, 139.75, 134.96, 133.44, 132.37, 132.32, 130.28, 129.35, 126.90, 126.45, 118.95, 118.43, 117.84, 104.62, 56.16, 52.23. HRMS (ESI) m/z: [M + Na]⁺ calcd for C₂₃H₂₀N₂O₆Na 443.1219; found 443.1249. |
| 4b | 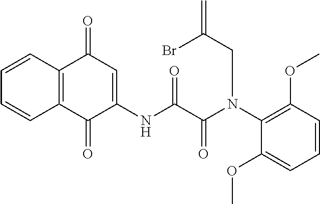 | ¹H NMR (500 MHz, CDCl₃) δ 9.83 (s, 1H), 8.16-8.11 (m, 1H), 8.09-8.04 (m, 1H), 7.81-7.68 (m, 2H), 7.65 (s, 1H), 7.29-7.24 (m, 1H), 6.60 (d, J = 8.5 Hz, 2H), 5.90 (d, J = 2.0 Hz, 1H), 5.53 (d, J = 1.9 Hz, 1H), 4.61 (s, 2H), 3.82 (s, 6H); ¹³C{¹H} NMR (126 MHz, CDCl₃) δ 185.15, 180.42, 162.01, 158.90, 155.38, 139.71, 135.02, 133.50, 132.31, 130.25, 129.53, 126.94, 126.48, 126.28, 119.51, 118.59, 117.97, 104.70, 56.68, 56.11. HRMS (ESI) m/z: [M + Na]⁺ calcd for C₂₃H₁₉BrN₂O₆Na 523.0307; found 523.0316. |
| 4c | 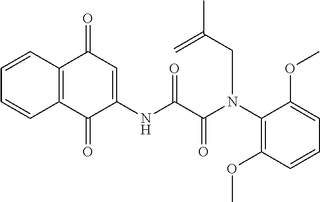 [SMILES: C=C(C)CN(C(=O)C(=O)NC1=CC(=O)c2ccccc2C1=O)c1c(OC)cccc1OC] | ¹H NMR (500 MHz, CDCl₃) δ 9.85 (s, 1H), 8.15-8.10 (m, 1H), 8.08-8.03 (m, 1H), 7.79-7.68 (m, 2H), 7.66 (s, 1H), 7.23 (d, J = 8.4 Hz, 1H), 6.58 (d, J = 8.4 Hz, 2H), 4.77-4.73 (m, 1H), 4.68 (s, 1H), 4.32 (s, 2H), 3.80 (s, 6H), 1.81 (s, 3H); ¹³C{¹H} NMR (126 MHz, CDCl₃) δ 185.19, 180.49, 162.00, 159.47, 155.70, 140.64, 139.78, 134.96, 133.44, 132.33, 130.28, 129.23, 126.90, 126.45, 118.80, 117.82, 114.61, 104.62, 56.06, 54.88, 20.82. HRMS (ESI) m/z: [M + Na]⁺ calcd for C₂₄H₂₂N₂O₆Na 457.1375; found 457.1347. |
| 4d | 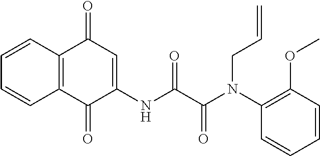 [SMILES: C=CCN(C(=O)C(=O)NC1=CC(=O)c2ccccc2C1=O)c1ccccc1OC] | ¹H NMR (500 MHz, CDCl₃) δ 9.90 (s, 1H), 8.13 (d, J = 7.6 Hz, 1H), 8.08-8.03 (m, 1H), 7.80-7.69 (m, 2H), 7.63 (d, J = 2.4 Hz, 1H), 7.35-7.25 (m, 1H), 7.19 (dd, J = 7.8, 1.7 Hz, 1H), 7.00 (t, J = 7.6 Hz, 1H), 6.92 (d, J = 8.3 Hz, 1H), 5.88 (ddt, J = 16.6, 10.0, 6.4 Hz, 1H), 5.21-5.14 (m, 2H), 4.49 (dd, J = 14.7, 6.2 Hz, 1H), 4.23 (dd, J = 14.8, 6.5 Hz, 1H), 3.77 (s, 3H); ¹³C{¹H} NMR (126 MHz, CDCl₃) δ 185.10, 180.41, 161.26, 159.08, 154.30, 139.65, 135.01, 133.49, 132.30, 131.76, 130.67, 130.23, 129.36, 127.60, 126.92, 126.48, 121.22, 118.88, 117.94, 111.86, 55.79, 53.84. HRMS (ESI) m/z: [M + Na]⁺ calcd for C₂₂H₁₈N₂O₅Na 413.1113; found 413.1111. |
| 4e | 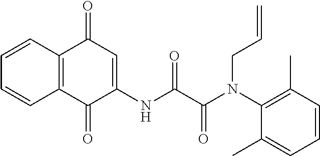 [SMILES: C=CCN(C(=O)C(=O)NC1=CC(=O)c2ccccc2C1=O)c1c(C)cccc1C] | ¹H NMR (500 MHz, CDCl₃) δ 10.12 (s, 1H), 8.15- 8.11 (m, 1H), 8.07-8.02 (m, 1H), 7.79-7.69 (m, 2H), 7.63 (s, 1H), 7.18 (m, 1H), 7.11 (d, J = 7.6 Hz, 2H), 5.98 (ddt, J = 16.9, 9.7, 7.0 Hz, 1H), 5.25-5.17 (m, 2H), 4.29 (d, J = 7.0 Hz, 2H), 2.22 (s, 6H); ¹³C{¹H} NMR (126 MHz, CDCl₃) δ 185.00, 180.37, 160.29, 158.31, 139.74, 139.45, 135.15, 135.03, 133.52, 132.27, 131.19, 130.21, 128.86, 128.36, 126.93, 126.48, 120.35, 118.19, 54.32, 18.72. HRMS (ESI) m/z: [M + Na]⁺ calcd for C₂₃H₂₀N₂O₄Na 411.1321; found 411.1358. |
| 4f | 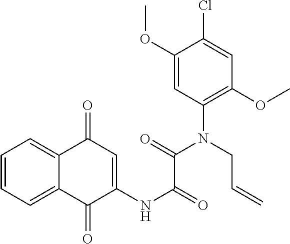 [SMILES: C=CCN(C(=O)C(=O)NC1=CC(=O)c2ccccc2C1=O)c1cc(OC)c(Cl)cc1OC] | ¹H NMR (500 MHz, CDCl₃) δ 9.88 (s, 1H), 8.14 (d, J = 7.6 Hz, 1H), 8.07 (d, J = 7.6 Hz, 1H), 7.81-7.70 (m, 2H), 7.63 (s, 1H), 6.95 (s, 1H), 6.82 (s, 1H), 5.88 (ddt, J = 16.9, 12.1, 6.4 Hz, 1H), 5.22-5.15 (m, 2H), 4.45 (dd, J = 14.9, 6.1 Hz, 1H), 4.22 (dd, J = 14.8, 6.6 Hz, 1H), 3.85 (s, 3H), 3.71 (s, 3H); ¹³C{¹H} NMR (126 MHz, CDCl₃) δ 185.01, 180.41, 161.28, 158.90, 149.52, 148.48, 139.57, 135.10, 133.55, 132.31, 131.66, 130.21, 129.59, 126.95, 126.55, 122.45, 119.22, 118.11, 114.33, 112.02, 57.02, 56.52, 54.00. HRMS (ESI) m/z: [M + Na]⁺ calcd for C₂₃H₁₉ClN₂O₆Na 477.0829; found 477.0842. |
| 4g | 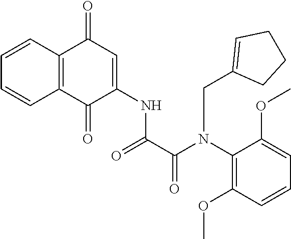 [SMILES: COc1cccc(OC)c1N(CC1=CCCC1)C(=O)C(=O)NC1=CC(=O)c2ccccc2C1=O] | ¹H NMR (500 MHz, CDCl₃) δ 9.86 (s, 1H), 8.13 (d, J = 7.5 Hz, 1H), 8.05 (d, J = 7.5 Hz, 1H), 7.79-7.68 (m, 2H), 7.65 (s, 1H), 7.23 (t, J = 8.3 Hz, 1H), 6.57 (d, J = 8.4 Hz, 2H), 5.41 (s, 1H), 4.39 (s, 2H), 3.79 (s, 6H), 2.33 (d, J = 7.8 Hz, 2H), 2.20 (d, J = 7.9 Hz, 2H), 1.82 (p, J = 7.6 Hz, 2H); ¹³C{¹H} NMR (126 MHz, CDCl₃) δ 185.20, 180.50, 161.83, 159.49, 155.68, 139.80, 139.26, 134.94, 133.43, 132.33, 130.28, 129.36, 129.15, 126.89, 126.43, 119.06, 117.77, 104.50, 56.04, 49.15, 34.06, 32.48, 23.87. HRMS (ESI) m/z: [M + Na]⁺ calcd for C₂₆H₂₄N₂O₆Na 483.1532; found 483.1569. |
| 4h | 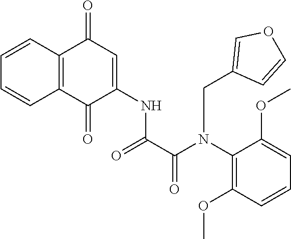 [SMILES: COc1cccc(OC)c1N(Cc1ccoc1)C(=O)C(=O)NC1=CC(=O)c2ccccc2C1=O] | ¹H NMR (500 MHz, CD 9.88 (s, 1H), 8.13 (d, J = 7.5 Hz, 1H), 8.08-8.03 (m, 1H), 7.79-7.68 (m, 2H), 7.65 (s, 1H), 7.29-7.19 (m, 3H), 6.54 (d, J = 8.4 Hz, 2H), 6.30 (d, J = 1.9 Hz, 1H), 4.72 (s, 2H), 3.70 (s, 6H); ¹³C{¹H} NMR (126 MHz, CDCl₃) δ 185.16, 180.50, 162.03, 159.30, 155.60, 142.53, 141.69, 139.74, 134.97, 133.44, 132.32, 130.27, 129.37, 126.89, 126.45, 119.85, 118.75, 117.87, 111.76, 104.67, 56.07, 43.26. HRMS (ESI) m/z: [M + Na]⁺ calcd for C₂₅H₂₀N₂O₇Na 483.1168; found 483.1185. |
| 4i | 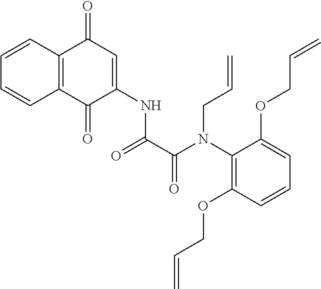 [SMILES: C=CCOc1cccc(OCC=C)c1N(CC=C)C(=O)C(=O)NC1=CC(=O)c2ccccc2C1=O] | ¹H NMR (500 MHz, CDCl₃) δ 9.89 (s, 1H), 8.13 (dd, J = 7.5, 1.5 Hz, 1H), 8.05 (dd, J = 7.5, 1.5 Hz, 1H), 7.81- 7.68 (m, 2H), 7.64 (s, 1H), 7.20 (t, J = 8.4 Hz, 1H), 6.57 (d, J = 8.4 Hz, 2H), 6.01-5.84 (m, 3H), 5.35 (q, J = 1.6 Hz, 1H), 5.32 (q, J = 1.7 Hz, 1H), 5.23 (q, J = 1.5 Hz, 1H), 5.21 (q, J = 1.4 Hz, 1H), 5.13 (dq, J = 17.1, 1.5 Hz, 1H), 5.07 (dq, J = 10.1, 1.2 Hz, 1H), 4.53 (dq, J = 4.9, 1.5 Hz, 4H), 4.36 (dt, J = 6.6, 1.3 Hz, 2H); ¹³C{¹H} NMR (126 MHz, CDCl₃) δ 185.21, 180.33, 161.85, 159.13, 154.53, 139.79, 134.92, 133.44, 132.73, 132.33, 132.30, 130.26, 129.03, 126.90, 126.43, 119.62, 118.58, 117.68, 117.64, 105.85, 69.51, 52.29. HRMS (ESI) m/z: [M + Na]⁺ calcd for C₂₇H₂₄N₂O₆Na 495.1532; found 495.1553. |
| 4j | 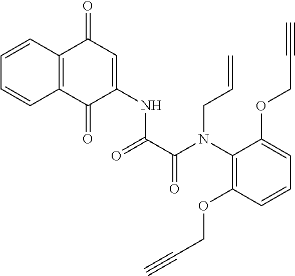 [SMILES: C#CCOc1cccc(OCC#C)c1N(CC=C)C(=O)C(=O)NC1=CC(=O)c2ccccc2C1=O] | ¹H NMR (500 MHz, CDCl₃) δ 9.91 (s, 1H), 8.13 (dd, J = 7.5, 1.5 Hz, 1H), 8.05 (dd, J = 7.5, 1.5 Hz, 1H), 7.81- 7.68 (m, 2H), 7.63 (s, 1H), 7.27 (t, J = 8.4 Hz, 1H), 6.74 (d, J = 8.5 Hz, 2H), 5.89 (ddt, J = 16.8, 10.1, 6.6 Hz, 1H), 5.14 (dq, J = 17.1, 1.4 Hz, 1H), 5.09 (dq, J = 10.1, 1.2 Hz, 1H), 4.70 (d, J = 2.4 Hz, 4H), 4.36 (dd, J = 6.7, 1.3 Hz, 2H), 2.44 (t, J = 2.4 Hz, 2H); ¹³C{¹H} NMR (126 MHz, CDCl₃) δ 185.22, 180.40, 161.73, 159.03, 153.54, 139.81, 134.94, 133.47, 132.28, 132.08, 130.28, 128.92, 126.91, 126.43, 120.30, 118.96, 117.86, 106.71, 77.99, 76.14, 56.52, 52.42. HRMS (ESI) m/z: [M + Na]⁺ calcd for C₂₇H₂₀N₂O₆Na 491.1219; found 491.1234. |
| 4k | 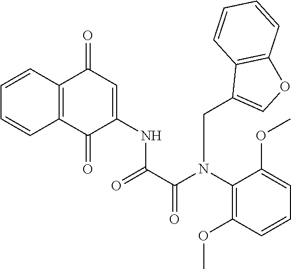 [SMILES: COc1cccc(OC)c1N(Cc1coc2ccccc12)C(=O)C(=O)NC1=CC(=O)c2ccccc2C1=O] | ¹H NMR (500 MHz, CDCl₃) δ 9.91 (s, 1H), 8.15-8.10 (m, 1H), 8.07-8.02 (m, 1H), 7.80-7.68 (m, 2H), 7.64 (s, 1H), 7.42-7.38 (m, 3H), 7.26-7.15 (m, 3H), 6.47 (d, J = 8.5 Hz, 2H), 5.00 (d, J = 0.8 Hz, 2H), 3.45 (s, 6H); ¹³C{¹H} NMR (126 MHz, CDCl₃) δ 185.13, 180.50, 161.91, 159.24, 155.79, 155.21, 144.63, 139.68, 134.97, 133.44, 132.30, 130.24, 129.55, 127.62, 126.87, 126.44, 124.27, 122.64, 120.54, 118.47, 117.92, 115.68, 111.18, 104.59, 55.83, 41.47; HRMS (ESI) m/z: [M + Na]⁺ calcd for C₂₉H₂₂N₂O₇Na 533.1324; found 533.1313. |
| 4l | 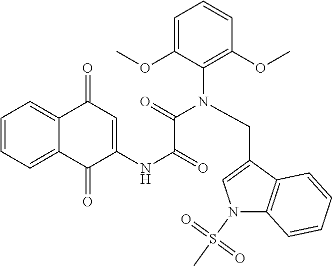 [SMILES: COc1cccc(OC)c1N(Cc1cn(S(C)(=O)=O)c2ccccc12)C(=O)C(=O)NC1=CC(=O)c2ccccc2C1=O] | ¹H NMR (500 MHz, CDCl₃) δ 9.92 (s, 1H), 8.16-8.10 (m, 1H), 8.05 (d, J = 7.4 Hz, 1H), 7.83 (d, J = 8.0 Hz, 1H), 7.79-7.69 (m, 2H), 7.62 (d, J = 14.3 Hz, 2H), 7.32 (dt, J = 17.4, 7.2 Hz, 2H), 7.21-7.14 (m, 2H), 6.45 (d, J = 8.4 Hz, 2H), 5.07 (s, 2H), 3.49 (s, 6H), 2.94 (s, 3H); ¹³C{¹H} NMR (126 MHz, CDCl₃) δ 185.14, 180.50, 161.85, 159.13, 155.70, 139.65, 135.08, 135.01, 133.48, 132.28, 130.47, 130.22, 129.61, 126.89, 126.47, 125.99, 125.08, 123.63, 120.79, 118.10, 117.97, 117.62, 112.90, 104.45, 55.86, 42.44, 40.45. |
| 4m | 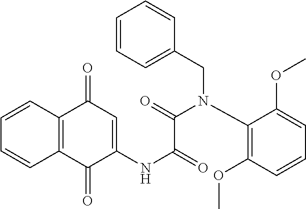 [SMILES: COc1cccc(OC)c1N(Cc1ccccc1)C(=O)C(=O)NC1=CC(=O)c2ccccc2C1=O] | ¹H NMR (500 MHz, CDCl₃) δ 9.93 (s, 1H), 8.18 (d, J = 7.6 Hz, 1H), 8.10 (d, J = 7.5 Hz, 1H), 7.84-7.73 (m, 2H), 7.70 (s, 1H), 7.33-7.30 (m, 3H), 7.29-7.22 (m, 3H), 6.54 (d, J = 8.3 Hz, 2H), 4.93 (s, 2H), 3.66 (s, 6H); ¹³C{¹H} NMR (126 MHz, CDCl₃) δ 185.17, 180.49, 162.00, 159.47, 155.62, 139.75, 136.16, 134.95, 133.43, 132.32, 130.26, 129.79, 129.38, 127.93, 127.63, 126.88, 126.43, 118.69, 117.87, 104.62, 56.04, 52.45. HRMS (ESI) m/z: [M + Na]⁺ calcd for C₂₇H₂₂N₂O₆Na 493.1375; found 493.1373. |
| 4n | 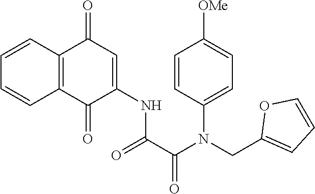 [SMILES: COc1ccc(N(Cc2ccco2)C(=O)C(=O)NC2=CC(=O)c3ccccc3C2=O)cc1] | ¹H NMR (500 MHz, CDCl₃) δ 10.01 (s, 1H), 8.15 (dd, J = 7.5, 1.6 Hz, 1H), 8.08 (dd, J = 7.6, 1.5 Hz, 1H), 7.83-7.71 (m, 2H), 7.64 (s, 1H), 7.41 (dd, J = 1.9, 0.8 Hz, 1H), 7.02-6.98 (m, 2H), 6.92-6.85 (m, 2H), 6.33 (dd, J = 3.2, 1.9 Hz, 1H), 6.24 (d, J = 3.3 Hz, 1H), 4.94 (s, 2H), 3.84 (s, 3H); ¹³C{¹H} NMR (126 MHz, CDCl₃) δ 185.03, 180.35, 160.31, 159.44, 158.76, 149.13, 142.85, 139.45, 135.05, 133.87, 133.54, 132.27, 130.18, 128.10, 126.95, 126.50, 118.17, 114.60, 110.64, 110.24, 55.51, 48.07. HRMS (ESI) m/z: [M + Na]⁺ calcd for C₂₄H₁₈N₂O₆Na 453.1063; found 453.1030. |
| 4o | 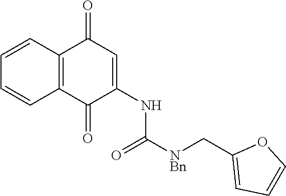 [SMILES: O=C1C=C(NC(=O)N(Cc2ccccc2)Cc2ccco2)C(=O)c2ccccc21] | ¹H NMR (500 MHz, CDCl₃) δ 8.19 (s, 1H), 8.09 (d, J = 7.7 Hz, 1H), 8.04 (d, J = 7.7 Hz, 1H), 7.75 (t, J = 7.5 Hz, 1H), 7.71-7.64 (m, 2H), 7.46-7.42 (m, 1H), 7.41-7.35 (m, 2H), 7.34-7.29 (m, 3H), 6.37 (d, J = 1.4 Hz, 2H), 4.67 (s, 2H), 4.54 (s, 2H); ¹³C{¹H} NMR (126 MHz, CDCl₃) δ 185.01, 181.58, 153.79, 149.9, 143.18, 141.73, 134.98, 133.02, 132.47, 130.19, 129.14, 128.16, 127.68, 126.65, 126.48, 115.26, 110.69, 109.22, 50.72, 43.72. HRMS (ESI) m/z: [M + H]⁺ calcd for C₂₃H₁₉N₂O₄387.1339; found 387.1343. |
| 4p | 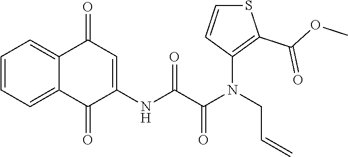 [SMILES: C=CCN(C(=O)C(=O)NC1=CC(=O)c2ccccc2C1=O)c1ccsc1C(=O)OC] | ¹H NMR (500 MHz, CDCl₃) δ 10.02 (s, 1H), 8.14 (dd, J = 7.6, 1.5 Hz, 1H), 8.06 (dd, J = 7.5, 1.5 Hz, 1H), 7.80-7.69 (m, 2H), 7.61 (s, 1H), 7.55 (d, J = 5.3 Hz, 1H), 7.06 (d, J = 5.3 Hz, 1H), 5.88 (ddt, J = 16.8, 10.3, 6.5 Hz, 1H), 5.21-5.12 (m, 2H), 4.42 (dd, J = 14.7, 6.7 Hz, 1H), 4.30 (dd, J = 14.8, 6.3 Hz, 1H), 3.78 (s, 3H); ¹³C{¹H} NMR (126 MHz, CDCl₃) δ 185.13, 180.23, 161.79, 160.61, 158.77, 144.36, 139.63, 134.99, 133.53, 132.27, 131.26, 130.58, 130.24, 127.45, 126.96, 126.47, 124.87, 119.56, 118.06, 53.96, 52.38. HRMS (ESI) m/z: [M + Na]⁺ calcd for C₂₁H₁₆N₂O₆SNa 447.0627; found 447.0659. |
| 4q | 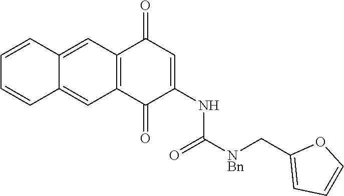 [SMILES: O=C1C=C(NC(=O)N(Cc2ccccc2)Cc2ccco2)C(=O)c2cc3ccccc3cc21] | ¹H NMR (500 MHz, CDCl₃) δ 8.63-8.56 (m, 2H), 8.32 (br s, 1H), 8.05 (d, J = 7.9 Hz, 1H), 8.02 (d, J = 7.6 Hz, 1H), 7.80 (s, 1H), 7.72-7.62 (m, 2H), 7.45 (dd, J = 1.8, 0.9 Hz, 1H), 7.43-7.36 (m, 2H), 7.36- 7.24 (m, 3H), 6.39 (dd, J = 3.3, 0.8 Hz, 1H), 6.38 (dd, J = 3.3, 1.8 Hz, 1H), 4.69 (s, 2H), 4.57 (br s, 2H). ¹³C NMR (126 MHz, CDCl₃) δ 184.7, 181.1, 153.8, 150.0, 143.2, 143.0, 135.7, 134.5, 130.4, 130.3, 130.0, 129.6, 129.4, 129.2, 128.6, 128.5, 128.2, 127.9, 127.7, 126.9, 117.0, 110.7, 109.3, 50.7, 43.7. HRMS (ESI) m/z: [M − H] ⁻ calcd for C₂₇H₁₉N₂O₄ 435.1350; found 435.1342. |
| 4r | 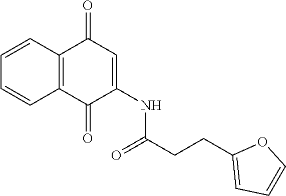 [SMILES: O=C(CCc1ccco1)NC1=CC(=O)c2ccccc2C1=O] | ¹H NMR (500 MHz, CDCl₃) δ 8.35 (br s, 1H), 8.10 (dd, J = 7.7, 1.1 Hz, 2H), 7.86 (s, 1H), 7.79 (td, J = 7.5, 1.4 Hz, 1H), 7.72 (td, J = 7.3, 1.1 Hz, 1H), 7.33 (d, J = 1.9 Hz, 1H), 6.29 (dd, J = 3.2, 2.0 Hz, 1H), 6.08 (d, J = 2.9 Hz, 1H), 3.08 (t, J = 7.3 Hz, 2H), 2.84 (t, J = 7.4 Hz, 2H). ¹³C NMR (126 MHz, CDCl₃) δ 185.4, 181.1, 171.3, 153.4, 141.6, 139.9, 135.1, 133.4, 132.2, 130.0, 126.8, 126.5, 117.4, 110.5, 106.1, 36.3, 23.4. HRMS (ESI) m/z: [M + H]⁺ calcd for C₁₇H₁₄NO₄296.0917; found 296.0923. |
| 1r | 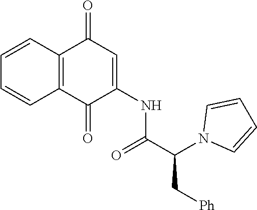 [SMILES: O=C1C=C(NC(=O)[C@H](Cc2ccccc2)n2cccc2)C(=O)c2ccccc21] | ¹H NMR (500 MHz, CDCl₃) δ 8.34 (s, 1H), 8.08 (ddd, J = 7.7, 1.3, 0.5 Hz, 1H), 8.02 (ddd, J = 7.7, 1.4, 0.5 Hz, 1H), 7.88 (s, 1H), 7.76 (td, J = 7.6, 1.4 Hz, 1H), 7.69 (td, J = 7.6, 1.3 Hz, 1H), 7.26-7.19 (m, 3H), 7.10-7.04 (m, 2H), 6.76 (t, J = 2.1 Hz, 2H), 6.37- 6.33 (m, 2H), 4.89 (dd, J = 10.5, 4.5 Hz, 1H), 3.72 (dd, J = 14.5, 4.5 Hz, 1H), 3.33 (dd, J = 14.5, 10.5 Hz, 1H). ¹³C NMR (126 MHz, CDCl₃) δ 185.2, 180.6, 170.1, 139.6, 136.7, 135.2, 133.5, 132.2, 129.9, 128.9, 128.8, 127.2, 126.9, 126.5, 120.3, 117.8, 110.9, 66.1, 37.8. HRMS (ESI) m/z: [M + H]⁺ calcd for C₂₃H₁₉N₂O₃ 371.1390; found 371.1391. |
| 1s | 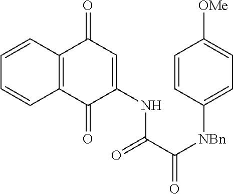 [SMILES: COc1ccc(N(Cc2ccccc2)C(=O)C(=O)NC2=CC(=O)c3ccccc3C2=O)cc1] | ¹H NMR (500 MHz, CDCl₃) δ 9.97 (br s, 1H), 8.13 (dd, J = 7.5, 1.2 Hz, 1H), 8.05 (dd, J = 7.6, 1.1 Hz, 1H), 7.77 (td, J = 7.5, 1.4 Hz, 1H), 7.72 (td, J = 7.5, 1.4 Hz, 1H), 7.62 (s, 1H), 7.33-7.27 (m, 3H), 7.25-7.20 (m, 2H), 6.94-6.88 (m, 2H), 6.85-6.79 (m, 2H), 4.94 (s, 2H), 3.79 (s, 3H). ¹³C NMR (126 MHz, CDCl₃) δ 185.0, 180.4, 160.5, 159.3, 159.1, 139.4, 135.8, 135.1, 134.0, 133.5, 132.2, 130.1, 129.2, 128.7, 128.2, 128.1, 126.9, 126.5, 118.1, 114.5, 55.6, 55.5. HRMS (ESI) m/z: [M + H]⁺ calcd for C₂₆H₂₁N₂O₅ 441.1445, found 441.1442. |
| 6a | 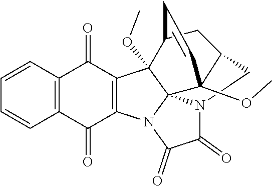 [SMILES: CO[C@@]12C=C[C@H]3C[C@@H]1CN1C(=O)C(=O)N4C5=C(C(=O)c6ccccc6C5=O)[C@]3(OC)[C@]142] | ¹H NMR (500 MHz, CDCl₃) δ 8.20-8.15 (m, 1H), 8.10-8.05 (m, 1H), 7.81-7.75 (m, 2H), 6.26 (t, J = 7.9 Hz, 1H), 6.20 (d, J = 8.9 Hz, 1H), 3.90 (dd, J = 11.3, 5.6 Hz, 1H), 3.78-3.73 (m, 1H), 3.41-3.35 (m, 4H), 3.33 (s, 3H), 2.68-2.59 (m, 1H), 2.15 (d, J = 13.4 Hz, 1H), 1.94 (t, J = 12.3 Hz, 1H); ¹³C{¹H} NMR (126 MHz, CDCl₃) δ 182.39, 176.53, 160.79, 160.09, 147.24, 134.66, 134.47, 134.37, 134.35, 132.08, 132.03, 127.34, 127.32, 126.64, 88.51, 86.07, 82.38, 53.39, 52.86, 49.94, 37.56, 32.92, 32.26. HRMS (ESI) m/z: [M + Na]⁺ calcd for C₂₃H₁₈N₂O₆Na 441.1063; found 441.1059. |
| 6b | 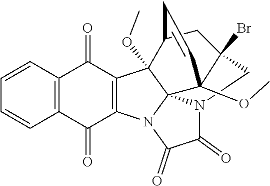 | ¹H NMR (500 MHz, CDCl₃) δ 8.22-8.15 (m, 1H), 8.13-8.05 (m, 1H), 7.85-7.76 (m, 2H), 6.44 (dd, J = 9.0, 7.0 Hz, 1H), 6.37 (dd, J = 9.0, 1.4 Hz, 1H), 4.18 (d, J = 11.0 Hz, 1H), 3.83 (d, J = 11.1 Hz, 1H), 3.71 (dtd, J = 7.0, 2.8, 1.3 Hz, 1H), 3.65 (s, 3H), 3.32 (s, 3H), 2.86 (dd, J = 14.5, 2.7 Hz, 1H), 2.46 (ddd, J = 14.5, 2.8, 1.3 Hz, 1H); ¹³C{¹H} NMR (126 MHz, CDCl₃) δ 182.16, 176.38, 160.08, 158.84, 147.02, 134.88, 134.57, 134.34, 134.19, 132.03, 132.01, 127.41, 126.81, 124.37, 87.89, 85.19, 84.25, 61.44, 58.04, 56.67, 53.42, 44.40, 33.96. HRMS (ESI) m/z: [M + Na]⁺ calcd for C₂₃H₁₇BrN₂O₆Na 521.0151; found 521.0172. |
| 6c | 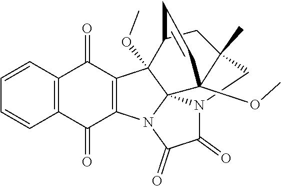 [SMILES: CO[C@@]12C=C[C@H]3C[C@]1(C)CN1C(=O)C(=O)N4C5=C(C(=O)c6ccccc6C5=O)[C@]3(OC)[C@]142] | ¹H NMR (500 MHz, CDCl₃) δ 8.19-8.13 (m, 1H), 8.08 (dd, J = 6.5, 3.0 Hz, 1H), 7.79 (dd, J = 9.1, 5.0 Hz, 2H), 6.31-6.26 (m, 2H), 3.72-3.67 (m, 1H), 3.64 (d, J = 11.0 Hz, 1H), 3.51 (s, 3H), 3.37 (d, J = 10.8 Hz, 1H), 3.32 (s, 3H), 2.31 (dd, J = 13.4, 2.9 Hz, 1H), 1.54-1.48 (m, 1H), 1.02 (s, 3H); ¹³C{¹H} NMR (126 MHz, CDCl₃) δ 182.34, 176.69, 160.31, 160.00, 146.93, 135.23, 134.71, 134.37, 133.78, 132.13, 132.01, 127.26, 126.71, 125.35, 89.86, 85.88, 85.02, 56.27, 55.52, 53.17, 47.73, 40.04, 33.05, 19.92. HRMS (ESI) m/z: [M + Na]⁺ calcd for C₂₄H₂₀N₂O₆Na 455.1219; found 455.1213. |
| 6d | 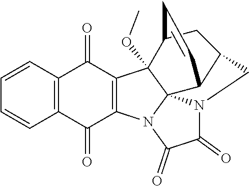 [SMILES: CO[C@]12C3=C(C(=O)c4ccccc4C3=O)N3C(=O)C(=O)N4C[C@H]5C[C@@H]1C=C[C@H]5[C@@]432] | ¹H NMR (500 MHz, CDCl₃) δ 8.20-8.14 (m, 1H), 8.10-8.05 (m, 1H), 7.82-7.75 (m, 2H), 6.25 (t, J = 7.7 Hz, 1H), 6.08 (t, J = 7.2 Hz, 1H), 3.91 (dd, J = 11.7, 5.5 Hz, 1H), 3.86-3.81 (m, 1H), 3.51 (d, J = 11.6 Hz, 1H), 3.34 (s, 3H), 2.73 (t, J = 5.3 Hz, 1H), 2.59 (dq, J = 11.0, 5.3 Hz, 1H), 2.06-1.97 (m, 1H), 1.89-1.81 (m, 1H); ¹³C{¹H} NMR (126 MHz, CDCl₃) δ 182.63, 176.95, 161.18, 158.53, 147.16, 136.07, 135.28, 134.67, 134.35, 132.09, 131.95, 127.81, 127.28, 126.69, 86.80, 85.83, 53.38, 50.93, 46.33, 35.37, 32.74, 30.70. HRMS (ESI) m/z: [M + Na]⁺ calcd for C₂₂H₁₆N₂O₅Na 411.0957; found 411.0955. |
| 6da | 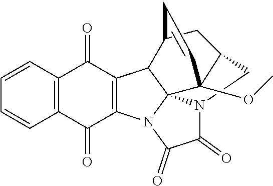 [SMILES: CO[C@@]12C=C[C@H]3C[C@@H]1CN1C(=O)C(=O)N4C5=C(C(=O)c6ccccc6C5=O)C3[C@]142] | ¹H NMR (500 MHz, CDCl₃) δ 8.15 (dd, J = 6.1, 3.1 Hz, 1H), 8.06-8.00 (m, 1H), 7.75 (dd, J = 5.9, 3.2 Hz, 2H), 6.31 (d, J = 8.8 Hz, 1H), 6.24 (dd, J = 8.9, 6.5 Hz, 1H), 3.87 (dd, J = 11.5, 5.5 Hz, 1H), 3.83 (d, J = 4.2 Hz, 1H), 3.69-3.63 (m, 1H), 3.43 (s, 3H), 3.26 (d, J = 11.5 Hz, 1H), 2.70-2.60 (m, 1H), 2.12 (ddd, J = 13.5, 10.9, 2.6 Hz, 1H), 1.88 (dd, J = 13.5, 2.8 Hz, 1H); ¹³C{¹H} NMR (126 MHz, CDCl₃) δ 182.20, 176.04, 162.15, 159.14, 145.84, 136.26, 135.17, 134.39, 134.22, 132.09, 131.98, 127.28, 126.31, 126.23, 89.15, 80.85, 52.90, 51.98, 49.83, 36.84, 36.06, 29.81. HRMS (ESI) calcd for C₂₄H₂₀N₂O₆Na⁺ (MNa+) 455.1219, found 455.1213; HRMS (ESI) m/z: [M + Na]⁺ calcd for C₂₂H₁₆N₂O₅Na 411.0957; found 411.0916. |
| 6e | 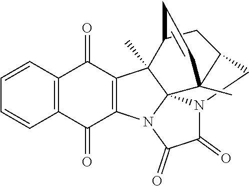 [SMILES: C[C@]12C3=C(C(=O)c4ccccc4C3=O)N3C(=O)C(=O)N4C[C@H]5C[C@@H]1C=C[C@@]5(C)[C@@]432] | ¹H NMR (500 MHz, CDCl₃) δ 8.20-8.15 (m, 1H), 8.08-8.03 (m, 1H), 7.79-7.76 (m, 2H), 6.34 (dd, J = 8.2, 6.8 Hz, 1H), 5.83 (dd, J = 8.2, 1.5 Hz, 1H), 3.88 (dd, J = 12.2, 5.6 Hz, 1H), 3.40 (dt, J = 7.8, 2.7 Hz, 1H), 3.35 (d, J = 12.2 Hz, 1H), 2.24 (m, 1H), 2.02- 1.90 (m, 2H), 1.30 (s, 3H), 1.10 (s, 3H); ¹³C{¹H} NMR (126 MHz, CDCl₃) δ 181.95, 176.41, 160.50, 160.17, 143.38, 143.30, 137.32, 134.35, 134.19, 132.27, 131.88, 131.83, 127.15, 126.39, 90.41, 53.44, 49.85, 45.77, 40.74, 35.87, 33.82, 18.88, 15.74. HRMS (ESI) m/z: [M + H]⁺ calcd for C₂₃H₁₉N₂O₄ 387.1339; found 387.1335. |
| 6f | 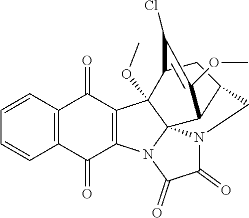 [SMILES: COC1=C(Cl)[C@H]2C[C@@H]3CN4C(=O)C(=O)N5C6=C(C(=O)c7ccccc7C6=O)[C@]2(OC)[C@]45[C@H]13] | ¹H NMR (500 MHz, CDCl₃) δ 8.24-8.20 (m, 1H), 8.19-8.14 (m, 1H), 7.88-7.80 (m, 2H), 3.97 (dd, J = 11.8, 5.3 Hz, 1H), 3.85 (t, J = 2.8 Hz, 1H), 3.68 (s, 3H), 3.54 (d, J = 11.7 Hz, 1H), 3.40 (s, 3H), 2.88- 2.84 (m, 1H), 2.89-2.80 (m, 1H), 2.24-2.15 (m, 1H), 2.13-2.05 (m, 1H); ¹³C{¹H} NMR (126 MHz, CDCl₃) δ 182.10, 176.69, 160.77, 158.04, 147.48, 147.36, 135.04, 134.89, 134.45, 132.10, 131.87, 127.32, 126.99, 105.73, 86.74, 86.68, 58.35, 54.08, 50.55, 49.51, 41.24, 36.13, 32.05. HRMS (ESI) m/z: [M + Na]⁺ calcd for C₂₃H₁₇ClN₂O₆Na 475.0673; found 475.0653. |
| 6g | 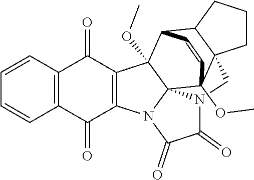 [SMILES: CO[C@@]12C=C[C@H]3C4CCC[C@]41CN1C(=O)C(=O)N4C5=C(C(=O)c6ccccc6C5=O)[C@]3(OC)[C@]142] | ¹H NMR (500 MHz, CDCl₃) δ 8.20-8.13 (m, 1H), 8.12-8.04 (m, 1H), 7.84-7.76 (m, 2H), 6.31 (d, J = 9.2 Hz, 1H), 6.21 (dd, J = 9.0, 6.9 Hz, 1H), 3.80 (q, J = 5.7 Hz, 2H), 3.51 (s, 3H), 3.42 (d, J = 10.7 Hz, 1H), 3.33 (s, 3H), 2.70 (td, J = 8.8, 2.9 Hz, 1H), 2.07-1.98 (m, 1H), 1.80-1.55 (m, 3H), 1.52-1.42 (m, 1H), 1.34-1.22 (m, 1H); ¹³C{¹H} NMR (126 MHz, CDCl₃) δ 182.38, 176.74, 160.58, 159.91, 146.71, 135.33, 134.70, 134.38, 133.64, 132.13, 132.01, 127.27, 126.73, 126.71, 89.95, 86.52, 85.35, 59.66, 55.93, 55.06, 53.17, 51.43, 37.39, 32.28, 32.19, 26.27. HRMS (ESI) m/z: [M + Na]⁺ calcd for C₂₆H₂₂N₂O₆Na 481.1375; found 481.1348. |
| 6h | 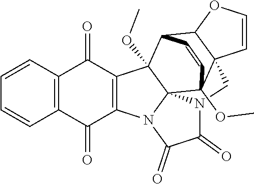 [SMILES: CO[C@@]12C=C[C@H]3C4OC=C[C@]41CN1C(=O)C(=O)N4C5=C(C(=O)c6ccccc6C5=O)[C@]3(OC)[C@]142] | . ¹H NMR (500 MHz, CDCl₃) δ 8.20-8.14 (m, 1H), 8.10 (dd, J = 6.4, 2.6 Hz, 1H), 7.85-7.77 (m, 2H), 6.37 (d, J = 2.7 Hz, 1H), 6.32 (d, J = 8.9 Hz, 1H), 6.09 (t, J = 7.9 Hz, 1H), 5.07 (d, J = 3.8 Hz, 1H), 4.89 (d, J = 2.8 Hz, 1H), 4.40 (dd, J = 6.9, 3.7 Hz, 1H), 4.03 (d, J = 11.2 Hz, 1H), 3.52 (s, 3H), 3.42 (d, J = 11.3 Hz, 1H), 3.38 (s, 3H); ¹³C{¹H} NMR (126 MHz, CDCl₃) δ 182.12, 176.54, 160.08, 159.18, 150.71, 147.17, 134.87, 134.52, 133.89, 132.03, 131.97, 129.98, 127.37, 126.83, 126.67, 98.35, 89.40, 88.72, 84.59, 84.42, 64.24, 55.93, 53.50, 51.88, 37.22. HRMS (ESI) m/z: [M + Na]⁺ calcd for C²⁵H¹⁸N²O⁷Na 481.1012; found 481.0965. |
| 6i | 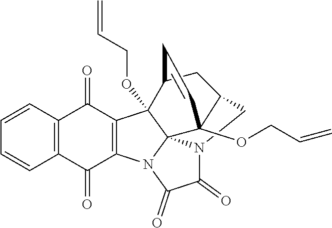 [SMILES: C=CCO[C@@]12C=C[C@H]3C[C@@H]1CN1C(=O)C(=O)N4C5=C(C(=O)c6ccccc6C5=O)[C@]3(OCC=C)[C@]142] | ¹H NMR (500 MHz, CDCl₃) δ 8.21-8.14 (m, 1H), 8.11-8.04 (m, 1H), 7.83-7.75 (m, 2H), 6.29-6.19 (m, 2H), 5.89-5.68 (m, 2H), 5.26 (dq, J = 17.2, 1.7 Hz, 1H), 5.18 (dt, J = 10.5, 1.5 Hz, 1H), 5.14 (dq, J = 17.3, 1.8 Hz, 1H), 5.04 (dq, J = 10.5, 1.5 Hz, 1H), 4.12 (ddt, J = 11.7, 5.1, 1.7 Hz, 3H), 4.03-3.91 (m, 2H), 3.76 (dq, J = 6.9, 2.2 Hz, 1H), 3.40 (d, J = 11.3 Hz, 1H), 2.60 (ddd, J = 11.1, 5.5, 1.8 Hz, 1H), 2.19 (dt, J = 13.4, 2.7 Hz, 1H), 1.95 (ddd, J = 13.4, 11.0, 2.4 Hz, 1H); ¹³C{¹H} NMR (126 MHz, CDCl₃) δ 182.28, 176.57, 160.62, 160.17, 147.12, 134.69, 134.39, 134.37, 133.86, 133.72, 132.08, 132.02, 127.74, 127.33, 126.66, 117.24, 117.02, 88.76, 85.69, 82.81, 67.20, 66.53, 50.12, 39.17, 33.77, 32.67. HRMS (ESI) m/z: [M + Na]⁺ calcd for C₂₇H₂₂N₂O₆Na 493.1375; found 493.1373. |
| 6j | 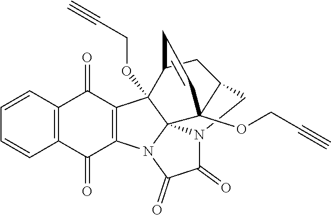 [SMILES: C#CCO[C@@]12C=C[C@H]3C[C@@H]1CN1C(=O)C(=O)N4C5=C(C(=O)c6ccccc6C5=O)[C@]3(OCC#C)[C@]142] | ¹H NMR (500 MHz, CDCl₃) δ 8.24-8.17 (m, 1H), 8.17-8.07 (m, 1H), 7.87-7.78 (m, 2H), 6.34 (dd, J = 8.9, 1.5 Hz, 1H), 6.30 (dd, J = 8.9, 6.8 Hz, 1H), 4.40- 4.23 (m, 4H), 4.11 (dd, J = 11.3, 5.6 Hz, 1H), 3.83 (ddt, J = 6.9, 3.4, 2.1 Hz, 1H), 3.45 (d, J = 11.4 Hz, 1H), 2.78 (ddd, J = 11.2, 5.5, 1.8 Hz, 1H), 2.51 (t, J = 2.4 Hz, 1H), 2.29 (dt, J = 15.9, 2.5 Hz, 2H), 2.00 (ddd, J = 13.4, 11.1, 2.3 Hz, 1H); ¹³C{¹H} NMR (126 MHz, CDCl₃) δ 182.48, 176.45, 160.37, 160.08, 147.35, 134.75, 134.55, 134.42, 133.77, 132.05, 132.01, 127.41, 126.68, 88.75, 86.38, 83.53, 79.90, 79.51, 75.99, 75.87, 54.52, 54.21, 50.07, 39.10, 33.96, 32.62. HRMS (ESI) m/z: [M + Na]⁺ calcd for C₂₇H₁₈N₂O₆Na 489.1063; found 489.1039. |
| 6k | 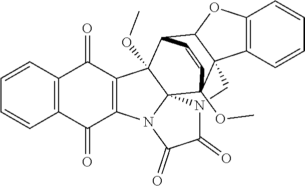 [SMILES: CO[C@@]12C=C[C@H]3C4Oc5ccccc5[C@]41CN1C(=O)C(=O)N4C5=C(C(=O)c6ccccc6C5=O)[C@]3(OC)[C@]142] | ¹H NMR (500 MHz, CDCl₃) δ 8.22-8.15 (m, 1H), 8.15-8.08 (m, 1H), 7.86-7.77 (m, 2H), 7.32 (dd, J = 7.5, 1.4 Hz, 1H), 7.24-7.17 (m, 1H), 6.88 (td, J = 7.5, 1.0 Hz, 1H), 6.74 (d, J = 8.0 Hz, 1H), 6.21-6.15 (m, 1H), 6.15-6.08 (m, 1H), 5.31 (dd, J = 4.0, 0.9 Hz, 1H), 4.56-4.50 (m, 1H), 4.32 (d, J = 11.4 Hz, 1H), 3.66 (d, J = 11.4 Hz, 1H), 3.53 (s, 3H), 3.43 (s, 3H). ¹³C{¹H} NMR (126 MHz, CDCl₃) δ 182.07, 176.52, 161.27, 160.22, 159.11, 147.18, 134.93, 134.57, 133.79, 132.04, 131.96, 130.68, 130.56, 127.39, 126.89, 126.07, 125.79, 122.78, 121.05, 109.68, 90.62, 89.25, 84.77, 84.41, 62.19, 56.46, 53.60, 52.35, 37.70. HRMS (ESI) m/z: [M + Na]⁺ calcd for C₂₉H₂₀N₂O₇Na 531.1168; found 531.1166. |
| 6l | 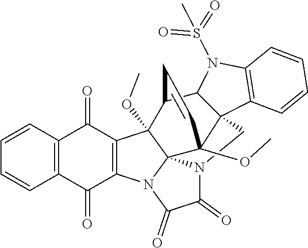 [SMILES: CO[C@@]12C=C[C@H]3C4N(S(C)(=O)=O)c5ccccc5[C@]41CN1C(=O)C(=O)N4C5=C(C(=O)c6ccccc6C5=O)[C@]3(OC)[C@]142] | ¹H NMR (500 MHz, DMSO-d₆) δ 8.11-8.02 (m, 2H), 7.93 (dd, J = 5.8, 3.3 Hz, 2H), 7.45 (d, J = 7.6 Hz, 1H), 7.36-7.29 (m, 1H), 7.25 (d, J = 8.1 Hz, 1H), 7.09 (t, J = 7.5 Hz, 1H), 6.32 (dd, J = 9.0, 1.4 Hz, 1H), 6.16 (dd, J = 9.0, 6.8 Hz, 1H), 4.97 (d, J = 3.4 Hz, 1H), 4.46 (dd, J = 7.2, 3.5 Hz, 1H), 4.23 (d, J = 11.2 Hz, 1H), 4.02 (d, J = 11.2 Hz, 1H), 3.50-3.40 (m, 6H), 3.11 (s, 3H); ¹³C{¹H} NMR (126 MHz, DMSO-d₆) δ 181.78, 175.86, 160.07, 158.24, 146.69, 143.77, 134.75, 134.52, 133.16, 131.79, 131.60, 130.71, 130.16, 126.80, 126.69, 126.50, 126.30, 126.21, 123.85, 113.23, 88.66, 84.24, 83.87, 70.64, 60.53, 56.18, 53.03, 52.05, 38.03, 34.31. |
| 6m | 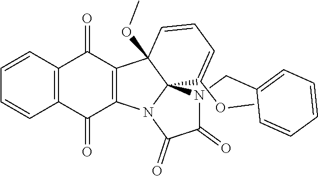 [SMILES: COC1=CC=C[C@@]2(OC)C3=C(C(=O)c4ccccc4C3=O)N3C(=O)C(=O)N(Cc4ccccc4)[C@@]132] | ¹H NMR (500 MHz, CDCl₃) δ 8.21 (dd, J = 6.1, 3.1 Hz, 1H), 8.10 (dd, J = 6.0, 3.2 Hz, 1H), 7.84-7.76 (m, 2H), 7.40-7.20 (m, 5H), 6.45 (d, J = 10.1 Hz, 1H), 6.06 (dd, J = 10.1, 6.5 Hz, 1H), 5.09-5.01 (m, 2H), 4.25 (d, J = 15.3 Hz, 1H), 3.35 (s, 3H), 2.96 (s, 3H); ¹³C{¹H} NMR (126 MHz, CDCl₃) δ 181.77, 176.69, 158.30, 157.71, 148.89, 145.79, 135.61, 135.31, 134.74, 134.28, 132.31, 131.77, 129.02, 128.18, 127.96, 127.30, 126.85, 122.38, 116.73, 100.19, 84.47, 83.66, 55.18, 53.00, 46.22. HRMS (ESI) m/z: [M + Na]⁺ calcd for C₂₇H₂₀N₂O₆Na 491.1219; found 491.1234. |
| 6n | 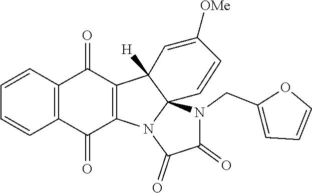 [SMILES: COC1=C[C@H]2C3=C(C(=O)c4ccccc4C3=O)N3C(=O)C(=O)N(Cc4ccco4)[C@]23C=C1] | ¹H NMR (500 MHz, CDCl₃) δ 8.16 (dd, J = 6.0, 3.1 Hz, 1H), 8.03 (dd, J = 6.0, 3.2 Hz, 1H), 7.79-7.72 (m, 2H), 7.38 (d, J = 1.9 Hz, 1H), 6.32 (dt, J = 15.5, 2.9 Hz, 3H), 5.39 (d, J = 10.0 Hz, 1H), 5.17 (dd, J = 6.5, 2.3 Hz, 1H), 5.09 (d, J = 15.6 Hz, 1H), 4.16 (d, J = 15.6 Hz, 1H), 4.04 (d, J = 6.5 Hz, 1H), 3.63 (s, 3H); ¹³C{¹H} NMR (126 MHz, CDCl₃) δ 181.82, 176.29, 157.69, 156.63, 150.54, 147.34, 143.14, 142.92, 137.48, 134.49, 134.23, 132.46, 132.14, 131.67, 127.27, 126.46, 123.66, 110.85, 110.66, 88.91, 81.48, 55.13, 45.82, 37.79. HRMS (ESI) m/z: [M + Na]⁺ calcd for C₂₄H₁₆N₂O₆Na 451.0906; found 451.0892. |
| 6o | 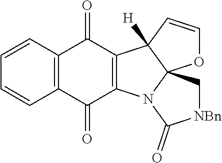 [SMILES: O=C1C2=C(C(=O)c3ccccc31)N1C(=O)N(Cc3ccccc3)C[C@@]13OC=C[C@@H]23] | ¹H NMR (500 MHz, CDCl₃) δ 8.16 (dd, J = 5.9, 3.1 Hz, 1H), 8.07-8.01 (m, 1H), 7.76-7.69 (m, 2H), 7.38- 7.27 (m, 5H), 6.39 (t, J = 2.5 Hz, 1H), 5.34 (t, J = 3.0 Hz, 1H), 4.59 (d, J = 14.9 Hz, 1H), 4.43 (d, J = 15.0 Hz, 1H), 4.41 (t, J = 2.6 Hz, 1H), 3.73 (d, J = 10.9 Hz, 1H), 3.62 (d, J = 11.0 Hz, 1H); ¹³C{¹H} NMR (126 MHz, CDCl₃) δ 182.35, 177.31, 154.83, 146.27, 146.03, 135.43, 134.15, 133.94, 133.79, 132.73, 132.32, 129.10, 128.48, 128.25, 127.42, 125.87, 104.54, 99.57, 55.19, 53.30, 47.90. HRMS (ESI) m/z: [M + Na]⁺ calcd for C₂₃H₁₆N₂O₄Na 407.1008; found 407.1014. |
| 6p | 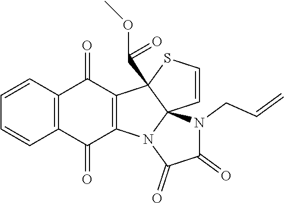 [SMILES: C=CCN1C(=O)C(=O)N2C3=C(C(=O)c4ccccc4C3=O)[C@]3(C(=O)OC)SC=C[C@]123] | ¹H NMR (500 MHz, CDCl₃) δ 8.27-8.17 (m, 1H), 8.09 (dd, J = 5.9, 3.0 Hz, 1H), 7.88-7.74 (m, 2H), 6.82 (d, J = 6.2 Hz, 1H), 5.82 (ddt, J = 16.5, 9.6, 6.2 Hz, 1H), 5.44 (d, J = 6.1 Hz, 1H), 5.36-5.24 (m, 2H), 4.07 (dd, J = 15.3, 6.3 Hz, 1H), 3.95 (dd, J = 15.3, 6.5 Hz, 1H), 3.75 (s, 3H); ¹³C{¹H} NMR (126 MHz, CDCl₃) δ 180.12, 176.38, 164.87, 158.21, 156.04, 144.68, 136.64, 135.63, 134.88, 134.61, 132.11, 131.86, 130.46, 127.56, 126.89, 120.82, 117.84, 99.60, 68.87, 54.79, 45.18; HRMS (ESI) m/z: [M + Na]⁺ calcd for C₂₁H₁₄N₂O₆SNa 445.0470; found 445.0472. |
| 7n | 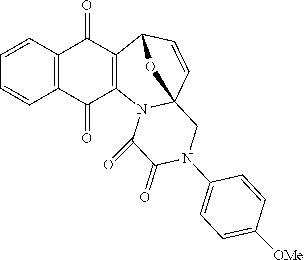 [SMILES: COc1ccc(N2C[C@]34C=C[C@H](O3)C3=C(C(=O)c5ccccc5C3=O)N4C(=O)C2=O)cc1] | ¹H NMR (500 MHz, CDCl₃) δ 8.10-8.03 (m, 2H), 7.78-7.71 (m, 2H), 7.34-7.28 (m, 2H), 6.99 (d, J = 8.7 Hz, 2H), 6.64-6.59 (m, 1H), 6.39 (d, J = 5.8 Hz, 1H), 5.91 (d, J = 1.9 Hz, 1H), 4.69 (d, J = 12.8 Hz, 1H), 4.00 (d, J = 12.9 Hz, 1H), 3.84 (s, 3H); ¹³C{¹H} NMR (126 MHz, CDCl₃) δ 181.00, 177.67, 159.21, 154.78, 153.61, 139.79, 134.79, 134.32, 133.99, 133.30, 133.11, 130.71, 129.62, 127.42, 126.38, 126.26, 125.49, 115.03, 95.04, 76.06, 55.73, 53.56. HRMS (ESI) m/z: [M + Na]⁺ calcd for C₂₄H₁₆N₂O₆Na 451.0906; found 451.0892. |
| 7o | 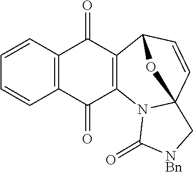 [SMILES: O=C1C2=C(C(=O)c3ccccc31)N1C(=O)N(Cc3ccccc3)C[C@@]13C=C[C@@H]2O3] | ¹H NMR (500 MHz, CDCl₃) δ 8.10-8.08 (m, 1H), 8.06-8.03 (m, 1H), 7.72-7.69 (m, 2H), 7.38-7.34 (m, 2H), 7.33-7.30 (m, 3H), 6.74 (dd, J = 5.6, 1.9 Hz, 1H), 5.82 (dd, J = 5.6, 0.5 Hz, 1H), 5.70 (d, J = 1.9 Hz, 1H), 4.62 (d, J = 15.0 Hz, 1H), 4.52 (d, J = 15.0 Hz, 1H), 3.86 (d, J = 11.3 Hz, 1H), 3.61 (d, J = 11.2 Hz, 1H); ¹³C{¹H} NMR (126 MHz, CDCl₃) δ 181.86, 179.36, 153.95, 139.43, 138.60, 135.44, 133.83, 133.70, 132.98, 131.10, 130.05, 129.06, 128.33, 128.16, 127.21, 125.97, 123.67, 96.10, 74.92, 49.02, 47.62. HRMS (ESI) m/z: [M + Na]⁺ calcd for C₂₃H₁₆N₂O₄Na 407.1008; found 407.1014. |
| 7q | 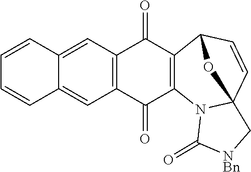 [SMILES: O=C1C2=C(C(=O)c3cc4ccccc4cc31)N1C(=O)N(Cc3ccccc3)C[C@@]13C=C[C@@H]2O3] | ¹H NMR (500 MHz, CDCl₃) δ 8.63 (s, 1H), 8.59 (s, 1H), 8.08-8.00 (m, 2H), 7.71-7.65 (m, 2H), 7.42-7.28 (m, 5H), 6.79 (dd, J = 5.6, 1.9 Hz, 1H), 5.85 (d, J = 5.7 Hz, 1H), 5.77 (d, J = 1.8 Hz, 1H), 4.65 (d, J = 15.0 Hz, 1H), 4.53 (d, J = 15.0 Hz, 1H), 3.88 (d, J = 11.2 Hz, 1H), 3.63 (d, J = 11.2 Hz, 1H). ¹³C NMR (126 MHz, CD₂Cl₂) δ 181.7, 179.3, 154.4, 140.2, 139.9, 136.3, 135.3, 135.2, 132.3, 130.6, 130.6, 130.0, 130.0, 129.8, 129.4, 129.4, 128.6, 128.5, 128.4, 128.4, 124.2, 96.6, 75.6, 49.5, 47.8. HRMS (ESI) m/z: [M + Na]⁺ calcd for C₂₇H₁₈N₂O₄Na 457.1164, found 457.1160. |
| 7r | 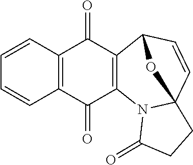 [SMILES: O=C1C2=C(C(=O)c3ccccc31)N1C(=O)CC[C@@]13C=C[C@@H]2O3] | ¹H NMR (500 MHz, CDCl₃) δ 8.09-8.06 (m, 1H), 8.06-8.03 (m, 1H), 7.75-7.69 (m, 2H), 6.73 (dd, J = 5.6, 1.9 Hz, 1H), 5.87 (d, J = 5.6 Hz, 1H), 5.71 (d, J = 1.9 Hz, 1H), 2.89-2.79 (m, 1H), 2.77-2.67 (m, 2H), 2.56-2.47 (m, 1H). ¹³C NMR (126 MHz, CDCl₃) δ 182.1, 178.9, 173.0, 138.9, 137.7, 134.0, 133.8, 132.9, 131.2, 131.0, 127.2, 126.5, 126.1, 101.3, 75.6, 29.3, 26.6. HRMS (ESI) m/z: [M + H]⁺ calcd for C₁₇H₁₂NO₄294.0761, found 294.0765. |
| 8a | 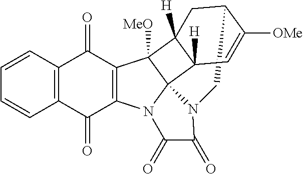 [SMILES: COC1=C[C@H]2[C@H]3C[C@@H]1CN1C(=O)C(=O)N4C5=C(C(=O)c6ccccc6C5=O)[C@]3(OC)[C@]214] | ¹H NMR (500 MHz, CDCl₃) δ 8.25-8.19 (m, 1H), 8.16 (dt, J = 7.0, 3.3 Hz, 1H), 7.83 (dq, J = 7.8, 3.9 Hz, 2H), 4.52 (d, J = 7.2 Hz, 1H), 4.24 (dd, J = 13.2, 4.5 Hz, 1H), 3.59-3.51 (m, 4H), 3.32-3.20 (m, 5H), 2.90 (dq, J = 4.9, 2.5 Hz, 1H), 2.44 (dd, J = 14.2, 2.5 Hz, 1H), 1.93 (ddd, J = 13.5, 7.8, 4.5 Hz, 1H); ¹³C{¹H} NMR (126 MHz, CDCl₃) δ 182.41, 177.06, 162.81, 157.51, 156.81, 146.57, 136.06, 134.93, 134.41, 132.49, 132.15, 127.48, 126.94, 86.18, 84.86, 82.65, 55.23, 54.49, 43.81, 39.82, 38.07, 37.31, 24.97. HRMS (ESI) m/z: [M + Na]⁺ calcd for C₂₃H₁₈N₂O₆Na 441.1063; found 441.1059. |
| 8c | 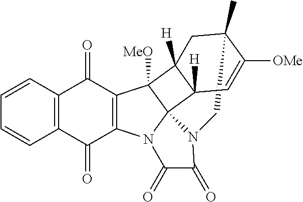 [SMILES: COC1=C[C@H]2[C@H]3C[C@]1(C)CN1C(=O)C(=O)N4C5=C(C(=O)c6ccccc6C5=O)[C@]3(OC)[C@]214] | ¹H NMR (500 MHz, CDCl₃) δ 8.25-8.20 (m, 1H), 8.20-8.14 (m, 1H), 7.83 (p, J = 7.5 Hz, 2H), 4.52- 4.47 (m, 1H), 4.03 (d, J = 13.1 Hz, 1H), 3.53 (s, 3H), 3.35 (d, J = 13.2 Hz, 1H), 3.29-3.20 (m, 5H), 2.30 (d, J = 14.2 Hz, 1H), 1.79 (dd, J = 14.4, 7.3 Hz, 1H), 1.31 (s, 3H); ¹³C{¹H} NMR (126 MHz, CDCl₃) δ 182.42, 177.04, 163.61, 157.28, 156.57, 146.57, 136.14, 134.94, 134.41, 132.49, 132.13, 127.48, 126.93, 85.91, 84.47, 82.63, 55.30, 54.49, 49.58, 39.94, 39.48, 38.07, 34.02, 21.60. HRMS (ESI) m/z: [M + Na]⁺ calcd for C₂₄H₂₀N₂O₆Na 455.1219; found 455.1213. |
| 9a | 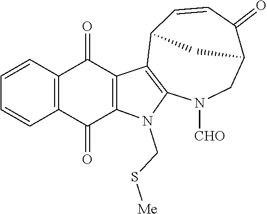 [SMILES: CSCn1c2c(c3c1N(C=O)C[C@H]1C[C@@H]3C=CC1=O)C(=O)c1ccccc1C2=O] | ¹H NMR (500 MHz, CDCl₃) δ 8.16 (tq, J = 7.3, 3.3 Hz, 2H), 7.99 (s, 1H), 7.72 (dp, J = 7.4, 4.4 Hz, 2H), 7.02 (m, 1H), 6.46 (d, J = 14.5 Hz, 1H), 6.07 (d, J = 9.9 Hz, 1H), 5.10 (t, J = 5.6 Hz, 1H), 4.76 (d, J = 14.4 Hz, 1H), 4.35 (dd, J = 14.4, 4.2 Hz, 1H), 3.57 (dd, J = 14.3, 1.9 Hz, 1H), 2.89 (d, J = 5.4 Hz, 1H), 2.70 (dt, J = 13.5, 5.6 Hz, 1H), 2.15 (q, J = 2.8 Hz, 1H), 2.11 (s, 3H); ¹³C{¹H} NMR (126 MHz, CDCl₃) δ 200.10, 182.19, 176.14, 161.99, 149.86, 136.07, 133.77, 133.65, 133.63, 133.53, 128.74, 128.59, 126.86, 126.56, 122.90, 118.31, 53.28, 48.33, 45.73, 30.63, 28.46, 15.04. HRMS (ESI) m/z: [M + Na]⁺ calcd for C₂₂H₁₈N₂O₄SNa 429.0885; found 429.0890. |
| 9g | 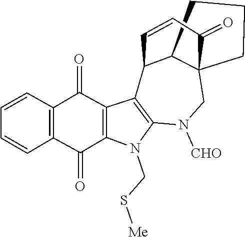 | ¹H NMR (500 MHz, CDCl₃) δ 8.21-8.12 (m, 2H), 7.93 (s, 1H), 7.77-7.68 (m, 2H), 6.89 (m, 1H), 6.46 (d, J = 14.3 Hz, 1H), 6.04 (d, J = 9.9 Hz, 1H), 5.07 (d, J = 6.0 Hz, 1H), 4.74 (d, J = 14.4 Hz, 1H), 4.26 (d, J = 13.9 Hz, 1H), 3.28 (d, J = 14.0 Hz, 1H), 2.54 (m, 1H), 2.36-2.23 (m, 1H), 2.13 (q, J = 5.6 Hz, 1H), 2.10 (s, 3H), 1.81-1.54 (m, 3H), 1.46-1.36 (m, 1H); ¹³C{¹H} NMR (126 MHz, CDCl₃) δ 202.75, 182.18, 176.13, 162.17, 147.16, 135.29, 133.83, 133.63, 133.56, 128.34, 127.73, 126.88, 126.55, 122.69, 119.24, 58.38, 57.46, 48.18, 48.09, 33.70, 31.80, 30.26, 20.70, 15.02. HRMS (ESI) m/z: [M + Na]⁺ calcd for C₂₅H₂₂N₂O₄SNa 469.1198; found 469.1194. |
| 10m | 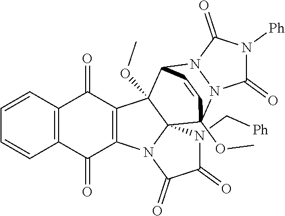 | ¹H NMR (500 MHz, CDCl₃) δ 8.16 (d, J = 7.4 Hz, 2H), 7.83-7.73 (m, 2H), 7.41-7.31 (m, 5H), 7.31-7.21 (m, 3H), 7.18-7.11 (m, 2H), 6.89 (dd, J = 8.7, 6.0 Hz, 1H), 6.15 (dd, J = 8.7, 1.5 Hz, 1H), 5.80 (dd, J = 5.9, 1.4 Hz, 1H), 5.12 (d, J = 15.6 Hz, 1H), 4.68 (d, J = 15.6 Hz, 1H), 3.49 (s, 3H), 3.28 (s, 3H); ¹³C{¹H} NMR (126 MHz, CDCl₃) δ 181.12, 175.69, 158.70, 157.85, 152.42, 151.78, 146.40, 135.39, 135.03, 134.50, 132.16, 132.12, 130.72, 129.54, 129.08, 128.84, 128.78, 128.46, 128.19, 127.59, 127.09, 125.02, 91.80, 89.44, 86.22, 56.25, 55.68, 52.60, 47.84, 29.88. HRMS (ESI) m/z: [M + Na]⁺ calcd for C₃₅H₂₅N₅O₈Na 666.1601; found 666.1584. |
| 11o | 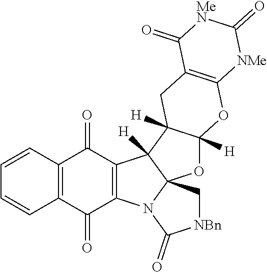 [SMILES: Cn1c2c(c(=O)n(C)c1=O)C[C@@H]1[C@H](O2)O[C@]23CN(Cc4ccccc4)C(=O)N2C2=C(C(=O)c4ccccc4C2=O)[C@H]13] | ¹H NMR (500 MHz, CDCl₃) δ 8.19-8.12 (m, 1H), 8.08-8.01 (m, 1H), 7.79-7.72 (m, 2H), 7.40-7.32 (m, 3H), 7.30-7.25 (m, 2H), 5.81 (d, J = 3.5 Hz, 1H), 4.60 (d, J = 14.9 Hz, 1H), 4.39 (d, J = 14.8 Hz, 1H), 3.66-3.57 (m, 3H), 3.34 (s, 3H), 3.29 (s, 3H), 2.97- 2.84 (m, 3H); ¹³C{¹H} NMR (126 MHz, CDCl₃) δ 182.09, 176.91, 162.59, 154.27, 153.79, 150.90, 146.22, 135.03, 134.25, 134.01, 132.56, 132.15, 131.93, 129.16, 128.48, 128.37, 127.45, 126.07, 102.70, 101.71, 84.21, 54.88, 53.86, 47.87, 40.96, 28.83, 28.27, 19.39. HRMS (ESI) m/z: [M + Na]⁺ calcd for C₃₀H₂₄N₄O₇Na 575.1543; found 575.1555. |
| 11h | 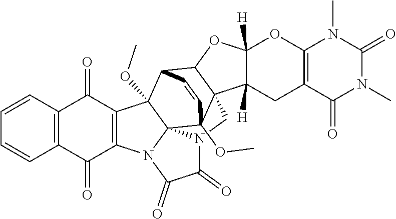 | ¹H NMR (500 MHz, CDCl₃) δ 8.21-8.16 (m, 1H), 8.13-8.08 (m, 1H), 7.85-7.80 (m, 2H), 6.36 (d, J = 8.9 Hz, 1H), 6.27 (t, J = 7.9 Hz, 1H), 5.67 (d, J = 3.7 Hz, 1H), 4.75 (d, J = 3.3 Hz, 1H), 4.30-4.25 (m, 1H), 3.84 (d, J = 11.1 Hz, 1H), 3.55 (d, J = 1.6 Hz, 3H), 3.40- 3.34 (m, 9H), 3.18 (d, J = 11.0 Hz, 1H), 2.86 (d, J = 17.8 Hz, 1H), 2.69 (dd, J = 17.9, 6.8 Hz, 1H), 2.51 (d, J = 6.2 Hz, 1H); ¹³C{¹H} NMR (126 MHz, CDCl₃) δ 182.14, 176.47, 162.26, 159.52, 159.33, 154.22, 151.04, 147.01, 134.98, 134.70, 133.92, 132.59, 131.96, 127.46, 126.89, 125.50, 104.45, 89.55, 89.05, 85.91, 84.69, 83.53, 58.12, 56.29, 53.48, 49.57, 39.98, 37.26, 28.87, 28.27, 18.12. HRMS (ESI) m/z: [M + Na]⁺ calcd for C₃₂H₂₆N₄O₁₀Na 649.1547; found 649.1570. |
| 12h | 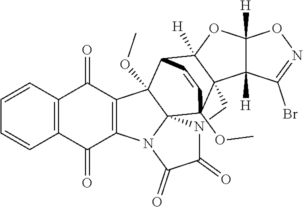 | ¹H NMR (500 MHz, CDCl₃) δ 8.21-8.16 (m, 1H), 8.14-8.07 (m, 1H), 7.85-7.80 (m, 2H), 6.35 (dd, J = 8.9, 1.5 Hz, 1H), 6.27 (dd, J = 8.9, 6.8 Hz, 1H), 6.05 (d, J = 5.9 Hz, 1H), 4.87 (d, J = 3.9 Hz, 1H), 4.69 (d, J = 11.4 Hz, 1H), 4.36-4.32 (m, 1H), 3.75 (d, J = 5.8 Hz, 1H), 3.63 (s, 3H), 3.51 (d, J = 11.5 Hz, 1H), 3.37 (s, 3H); ¹³C{¹H} NMR (126 MHz, CDCl₃) δ 182.02, 176.53, 159.49, 159.18, 147.02, 137.94, 135.04, 134.70, 134.35, 133.84, 131.97, 131.92, 127.43, 126.95, 124.08, 111.36, 88.93, 88.59, 83.83, 83.75, 62.90, 58.18, 56.78, 53.59, 47.76, 38.76. HRMS (ESI) m/z: [M + Na]⁺ calcd for C₂₆H₁₈BrN₃O₈Na 604.0159; found 604.0107. |
| 13h | 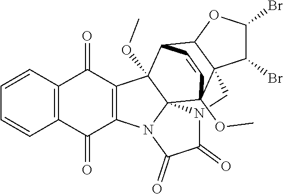 | ¹H NMR (500 MHz, CDCl₃) δ 8.21-8.15 (m, 1H), 8.12-8.07 (m, 1H), 7.86-7.79 (m, 2H), 6.51 (d, J = 4.2 Hz, 1H), 6.35 (d, J = 8.9 Hz, 1H), 6.26 (dd, J = 9.0, 6.6 Hz, 1H), 4.99 (d, J = 3.6 Hz, 1H), 4.51 (d, J = 11.5 Hz, 1H), 4.33-4.27 (m, 1H), 4.20 (d, J = 4.1 Hz, 1H), 3.80 (d, J = 11.5 Hz, 1H), 3.55 (s, 3H), 3.39 (s, 3H); ¹³C{¹H} NMR (126 MHz, CDCl₃) δ 182.05, 176.41, 159.72, 159.09, 147.10, 135.01, 134.70, 133.57, 133.39, 131.93, 127.46, 126.89, 125.05, 93.76, 89.25, 89.12, 85.54, 84.22, 57.98, 56.31, 53.61, 52.03, 50.25, 36.85. |
| 13a | 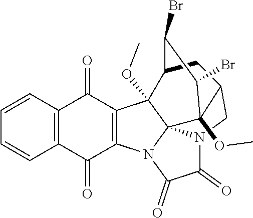 | ¹H NMR (500 MHz, CDCl₃) δ 8.25-8.19 (m, 1H), 8.19-8.13 (m, 1H), 7.89-7.80 (m, 2H), 4.54 (d, J = 3.8 Hz, 1H), 4.40-4.34 (m, 1H), 3.87 (dd, J = 11.2, 5.3 Hz, 1H), 3.35-3.26 (m, 4H), 3.22 (s, 3H), 3.16- 3.11 (m, 1H), 2.98-2.84 (m, 2H), 2.15-2.09 (m, 1H); ¹³C{¹H} NMR (126 MHz, CDCl₃) δ 182.98, 175.99, 160.83, 159.22, 149.30, 134.95, 134.74, 132.05, 131.95, 127.69, 127.06, 87.60, 84.68, 78.17, 53.59, 53.56, 51.09, 50.07, 49.92, 37.54, 34.83, 27.39. HRMS (ESI) m/z: [M + Na]⁺ calcd for C₂₃H₁₈Br₂N₂O₈Na 600.9410; found 600.9362. |
| 10s | 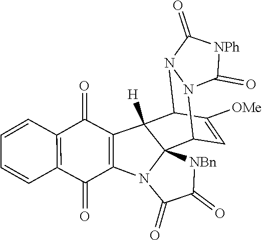 | ¹H NMR (500 MHz, CDCl₃) δ 8.25-8.15 (m, 1H), 8.14-8.04 (m, 1H), 7.84-7.72 (m, 2H), 7.42-7.28 (m, 8H), 7.14 (d, J = 7.4 Hz, 2H), 5.52 (t, J = 3.2 Hz, 1H), 4.98 (d, J = 6.4 Hz, 1H), 4.90 (d, J = 15.7 Hz, 1H), 4.82 (d, J = 15.6 Hz, 1H), 4.77 (dd, J = 6.5, 2.8 Hz, 1H), 3.83 (d, J = 3.8 Hz, 1H), 3.67 (s, 3H). ¹³C NMR (126 MHz, CDCl₃) δ 181.5, 176.0, 158.6, 158.5, 157.2, 156.5, 155.9, 145.2, 134.6, 134.5, 134.5, 132.7, 132.3, 131.9, 130.8, 129.6, 129.2, 128.7, 128.7, 127.5, 126.7, 126.7, 125.3, 89.6, 84.7, 61.5, 56.4, 54.6, 47.3, 46.4. HRMS (ESI) m/z: [M + H]⁺ calcd for C³⁴H²⁴N⁵O⁷ 614.1670, found 614.1680. |

### REFERENCES

1. Sunassee, S. N., et al., “Cytotoxic and Antioxidant Marine Prenylated Quinones and Hydroquinones,” Nat. Prod. Rep. 2012, 29, 513-535.
2. Son, E. J., et al., “Quinone and Its Derivatives for Energy Harvesting and Storage Materials,” J. Mater. Chem. A 2016, 4, 11179-11202.
3. Inks, E. S., et al., “A Novel Class of Small Molecule Inhibitors of HDAC6,” ACS Chem. Biol. 2012, 7, 331-339.
4. Josey, B. J., et al., “Structure-Activity Relationship Study of Vitamin K Derivatives Yields Highly Potent Neuroprotective Agents,” J. Med. Chem. 2013, 56, 1007-1022.
5. Mfuh, A. M., et al., “Concise Total Synthesis of Trichodermamides A, B, and C Enabled by an Efficient Construction of the 1,2-Oxazadecaline Core,” J. Am. Chem. Soc. 2015, 137, 8050-8053.
6. Zhang, Z.; Chen, J.; Yang, Z.; Tang, Y. Rapid Biomimetic Total Synthesis of (±)-Rossinone B. Org. Lett. 2010, 12, 5554-5557.
7. Usui, I.; Lin, D. W.; Masuda, T.; Baran, P. S. Convergent Synthesis and Structural Confirmation of Phellodonin and Sarcodonin E. Org. Lett. 2013, 15, 2080-2083.
8. Ryu, C.-K., et al., Bioorg. Med. Chem. Lett. 2009, 19, 146-148
9. Benites, J., et al., Eur. J. Med. Chem. 2010, 45, 6052-6057
10. Prachayasittikul, V., et al., Eur. J. Med. Chem. 2014, 84, 247-263.

That which is claimed is:

1. A method of producing a hydroquinone cycloaddition product, comprising a photo-induced intramolecular cycloaddition reaction to produce a photoproduct, wherein the photoproduct is selected from the group consisting of:

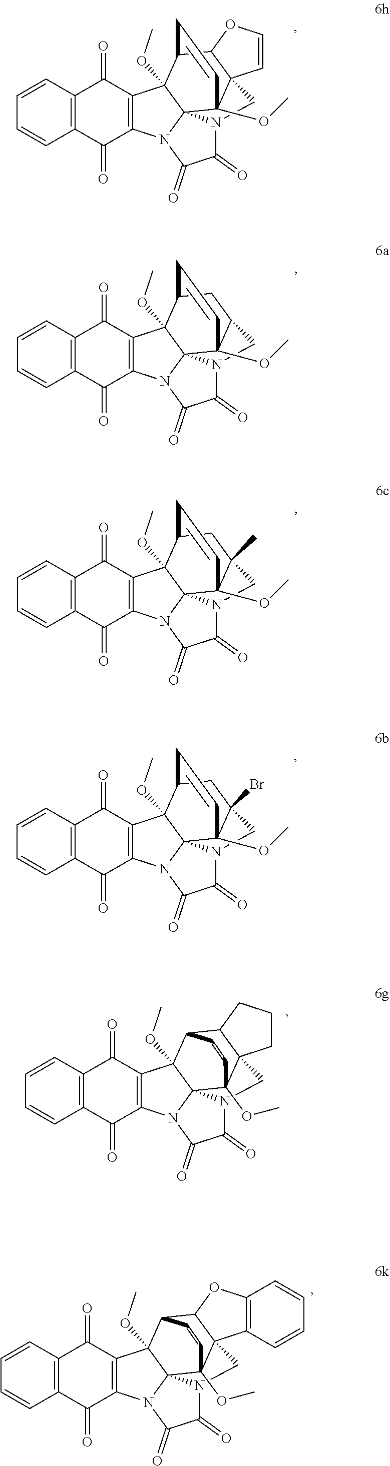

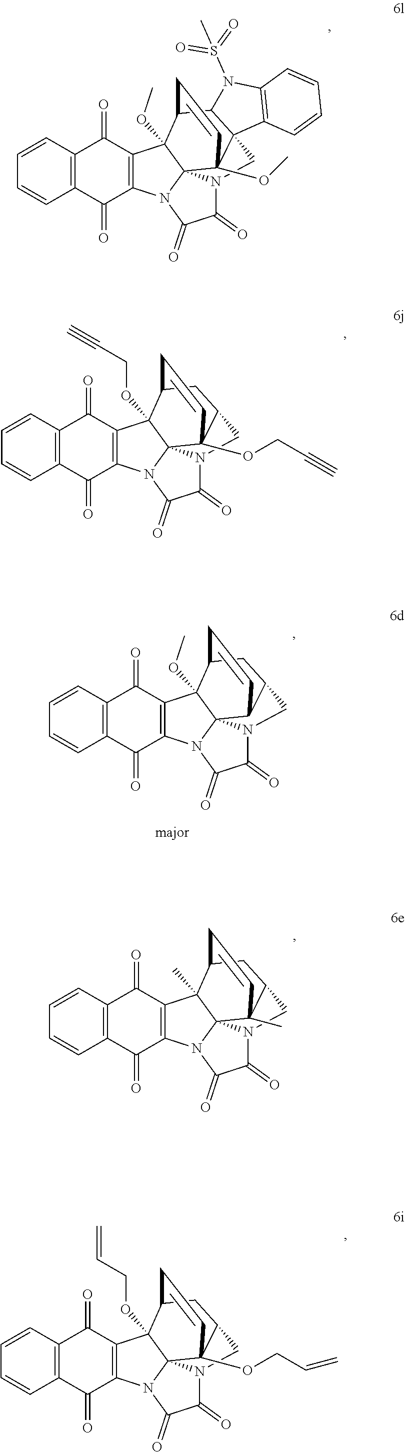 [SMILES: CO[C@@]12C=C[C@H]3C4N(S(C)(=O)=O)c5ccccc5[C@]41CN1C(=O)C(=O)N4C5=C(C(=O)c6ccccc6C5=O)[C@]3(OC)[C@]142 ; C#CCO[C@@]12C=C[C@H]3C[C@@H]1CN1C(=O)C(=O)N4C5=C(C(=O)c6ccccc6C5=O)[C@]3(OCC#C)[C@]142 ; CO[C@]12C3=C(C(=O)c4ccccc4C3=O)N3C(=O)C(=O)N4C[C@H]5C[C@@H]1C=C[C@H]5[C@@]432 ; C[C@]12C3=C(C(=O)c4ccccc4C3=O)N3C(=O)C(=O)N4C[C@H]5C[C@@H]1C=C[C@@]5(C)[C@@]432 ; C=CCO[C@@]12C=C[C@H]3C[C@@H]1CN1C(=O)C(=O)N4C5=C(C(=O)c6ccccc6C5=O)[C@]3(OCC=C)[C@]142]

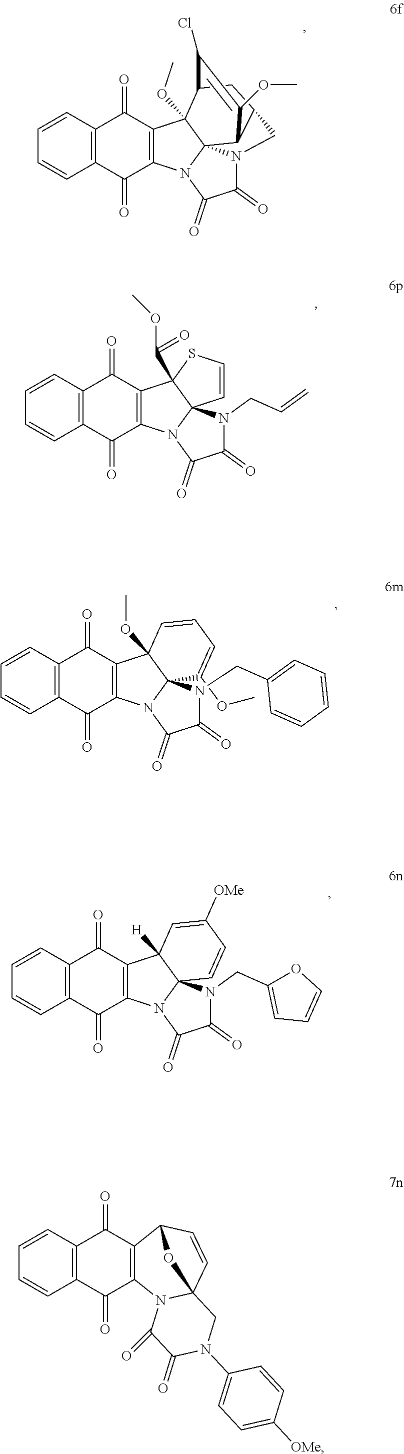 [SMILES: COC1=C(Cl)[C@H]2C[C@@H]3CN4C(=O)C(=O)N5C6=C(C(=O)c7ccccc7C6=O)[C@]2(OC)[C@]45[C@H]13 ; C=CCN1C(=O)C(=O)N2C3=C(C(=O)c4ccccc4C3=O)[C@]3(C(=O)OC)SC=C[C@]123 ; COC1=CC=C[C@@]2(OC)C3=C(C(=O)c4ccccc4C3=O)N3C(=O)C(=O)N(Cc4ccccc4)[C@@]132 ; COC1=C[C@H]2C3=C(C(=O)c4ccccc4C3=O)N3C(=O)C(=O)N(Cc4ccco4)[C@]23C=C1 ; COc1ccc(N2C[C@]34C=C[C@H](O3)C3=C(C(=O)c5ccccc5C3=O)N4C(=O)C2=O)cc1]

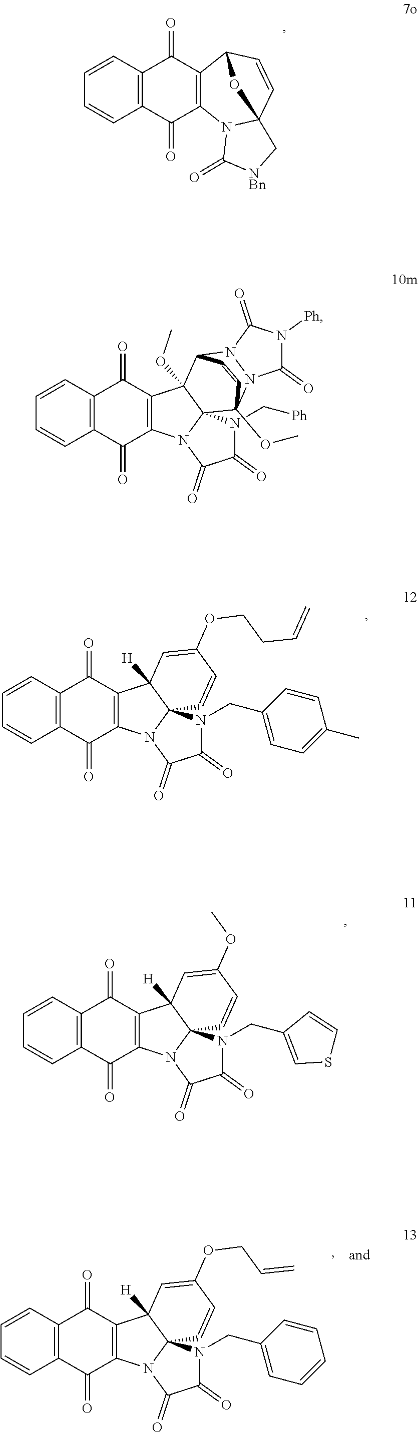 [SMILES: O=C1C2=C(C(=O)c3ccccc31)N1C(=O)N(Cc3ccccc3)C[C@@]13C=C[C@@H]2O3 ; CO[C@@]12C3=C(C(=O)c4ccccc4C3=O)N3C(=O)C(=O)N(Cc4ccccc4)[C@]31[C@]1(OC)C=C[C@H]2n2c(=O)n(-c3ccccc3)c(=O)n21 ; C=CCCOC1=C[C@H]2C3=C(C(=O)c4ccccc4C3=O)N3C(=O)C(=O)N(Cc4ccc(C)cc4)[C@]23C=C1 ; COC1=C[C@H]2C3=C(C(=O)c4ccccc4C3=O)N3C(=O)C(=O)N(Cc4ccsc4)[C@]23C=C1 ; C=CCOC1=C[C@H]2C3=C(C(=O)c4ccccc4C3=O)N3C(=O)C(=O)N(Cc4ccccc4)[C@]23C=C1]

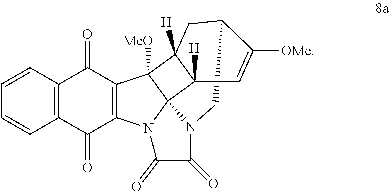 [SMILES: COC1=C[C@H]2[C@H]3C[C@@H]1CN1C(=O)C(=O)N4C5=C(C(=O)c6ccccc6C5=O)[C@]3(OC)[C@]214]

2. The method of claim 1, wherein the photoproduct is selected from the group consisting of:

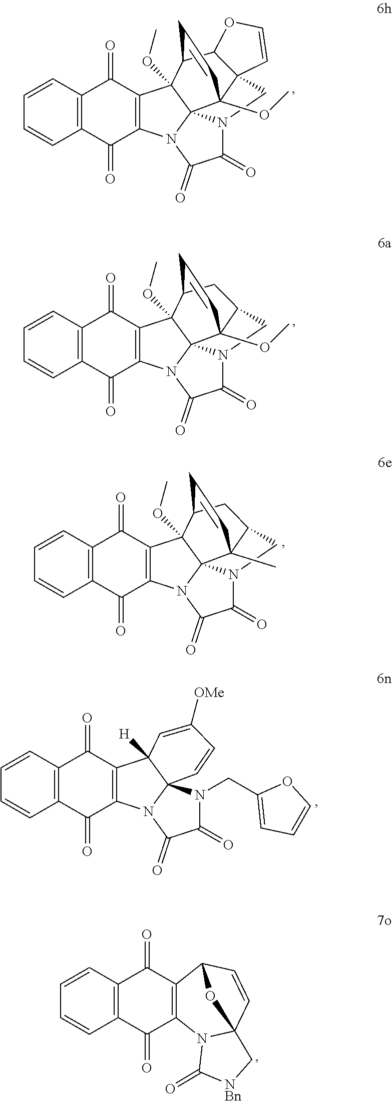

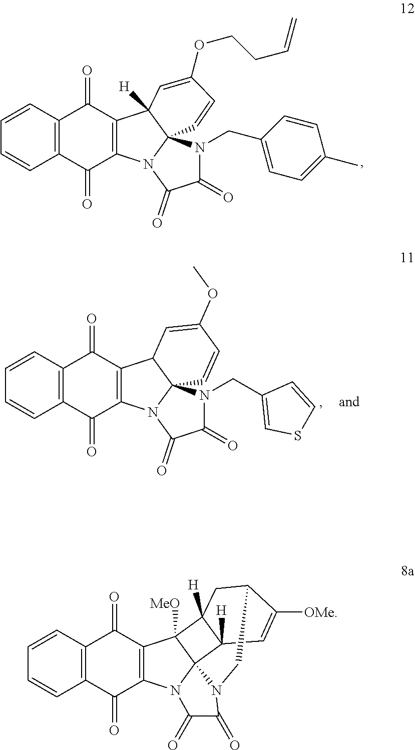

3. The method of claim 1, wherein the photo-induced intramolecular cycloaddition reaction is conducted in a solvent selected from dichloromethane (DCM) and dimethyl sulfoxide (DMSO).

4. The method of claim 3, wherein the solvent is dimethyl sulfoxide (DMSO).

5. The method of claim 1, wherein the photo-induced intramolecular cycloaddition reaction is induced with irradiation with an ultraviolet (UV) light source.

6. The method of claim 5, wherein the ultraviolet (UV) light source is selected from the group consisting of a light emitting diode (LED) array emitting light in a 410-420 nm wavelength, a mercury UV lamp, solar radiation.

7. The method of claim 6, wherein the ultraviolet (UV) light source is a light emitting diode (LED) array.

8. The method of claim 7, wherein the light emitting diode (LED) array emits light with a lambda max (λₘₐₓ) of 410-420 nm wavelength.

9. The method of claim 1, wherein the photo-induced intramolecular cycloaddition reaction is conducted at room temperature (25±5° C.).

10. The method of claim 1, wherein the photoproduct is obtained in a yield of at least 60%.

11. The method of claim 3, wherein a flow rate of 8-13 mL per hour is used.

12. The method of claim 1, wherein the compound is

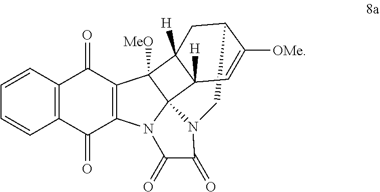 [SMILES: COC1=C[C@H]2[C@H]3C[C@@H]1CN1C(=O)C(=O)N4C5=C(C(=O)c6ccccc6C5=O)[C@]3(OC)[C@]214]

13. A compound selected from the group consisting of:

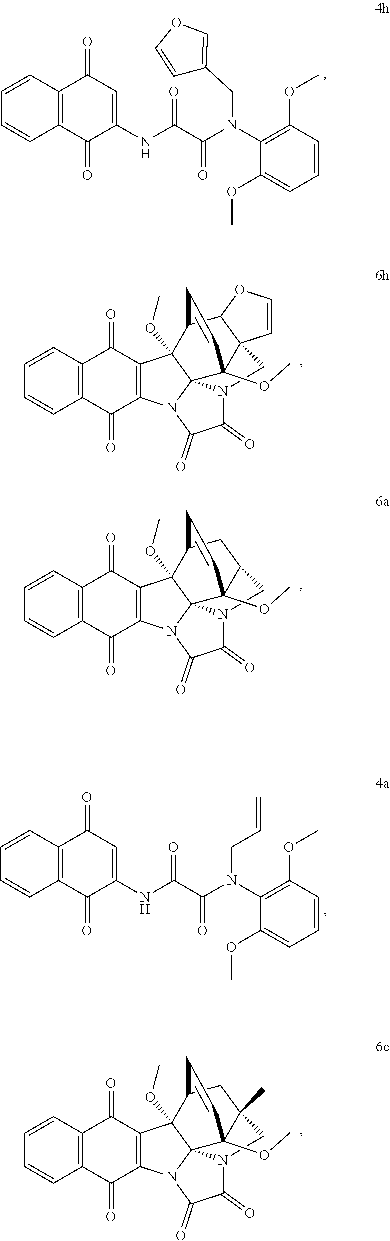 [SMILES: COc1cccc(OC)c1N(Cc1ccoc1)C(=O)C(=O)NC1=CC(=O)c2ccccc2C1=O ; CO[C@@]12C=C[C@H]3C4OC=C[C@]41CN1C(=O)C(=O)N4C5=C(C(=O)c6ccccc6C5=O)[C@]3(OC)[C@]142 ; CO[C@@]12C=C[C@H]3C[C@@H]1CN1C(=O)C(=O)N4C5=C(C(=O)c6ccccc6C5=O)[C@]3(OC)[C@]142 ; C=CCN(C(=O)C(=O)NC1=CC(=O)c2ccccc2C1=O)c1c(OC)cccc1OC ; CO[C@@]12C=C[C@H]3C[C@]1(C)CN1C(=O)C(=O)N4C5=C(C(=O)c6ccccc6C5=O)[C@]3(OC)[C@]142]

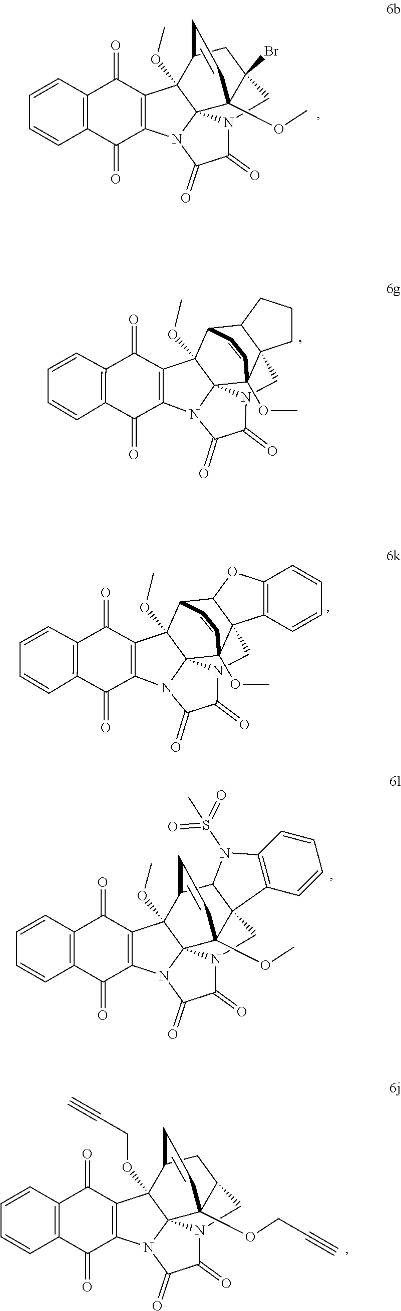 [SMILES: CO[C@@]12C=C[C@H]3C[C@]1(Br)CN1C(=O)C(=O)N4C5=C(C(=O)c6ccccc6C5=O)[C@]3(OC)[C@]142 ; CO[C@@]12C=C[C@H]3C4CCC[C@]41CN1C(=O)C(=O)N4C5=C(C(=O)c6ccccc6C5=O)[C@]3(OC)[C@]142 ; CO[C@@]12C=C[C@H]3C4Oc5ccccc5[C@]41CN1C(=O)C(=O)N4C5=C(C(=O)c6ccccc6C5=O)[C@]3(OC)[C@]142 ; CO[C@@]12C=C[C@H]3C4N(S(C)(=O)=O)c5ccccc5[C@]41CN1C(=O)C(=O)N4C5=C(C(=O)c6ccccc6C5=O)[C@]3(OC)[C@]142 ; C#CCO[C@@]12C=C[C@H]3C[C@@H]1CN1C(=O)C(=O)N4C5=C(C(=O)c6ccccc6C5=O)[C@]3(OCC#C)[C@]142]

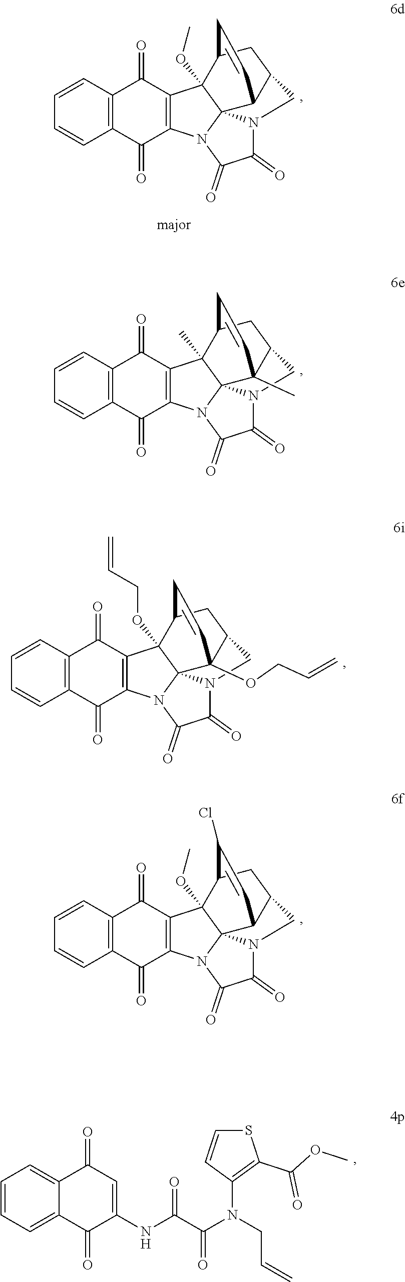 [SMILES: CO[C@]12C3=C(C(=O)c4ccccc4C3=O)N3C(=O)C(=O)N4C[C@H]5C[C@@H]1C=C[C@H]5[C@@]432 ; C[C@]12C3=C(C(=O)c4ccccc4C3=O)N3C(=O)C(=O)N4C[C@H]5C[C@@H]1C=C[C@@]5(C)[C@@]432 ; C=CCO[C@@]12C=C[C@H]3C[C@@H]1CN1C(=O)C(=O)N4C5=C(C(=O)c6ccccc6C5=O)[C@]3(OCC=C)[C@]142 ; CO[C@]12C3=C(C(=O)c4ccccc4C3=O)N3C(=O)C(=O)N4C[C@H]5C[C@@H]1C(Cl)=C[C@H]5[C@@]432 ; C=CCN(C(=O)C(=O)NC1=CC(=O)c2ccccc2C1=O)c1ccsc1C(=O)OC]

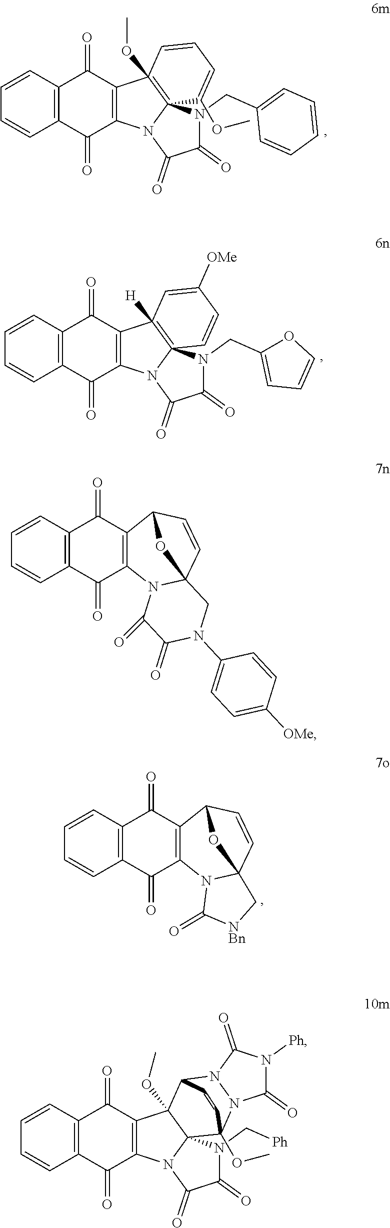

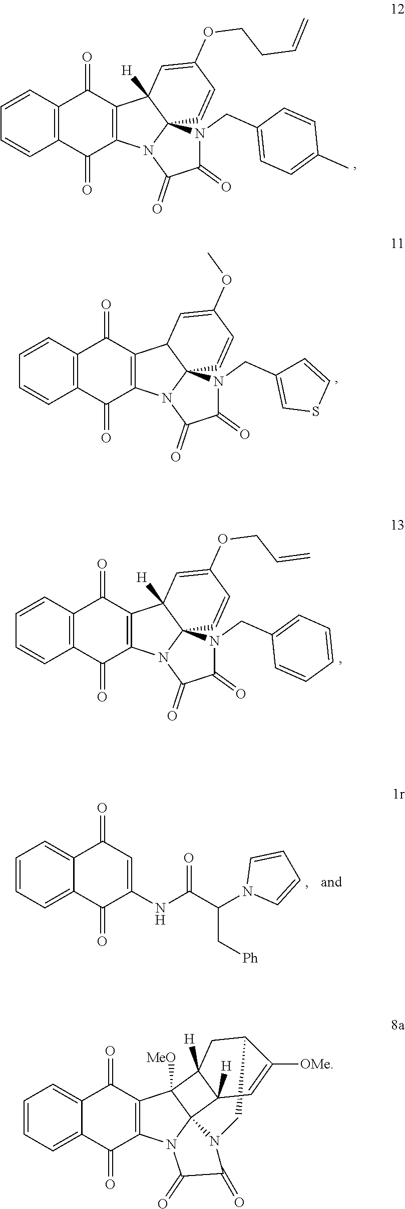

14. The compound of claim 13, wherein the compound is selected from the group consisting of:

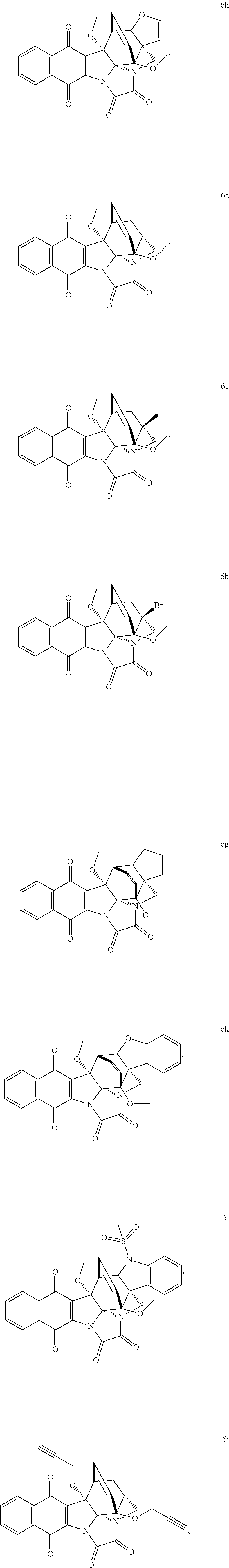

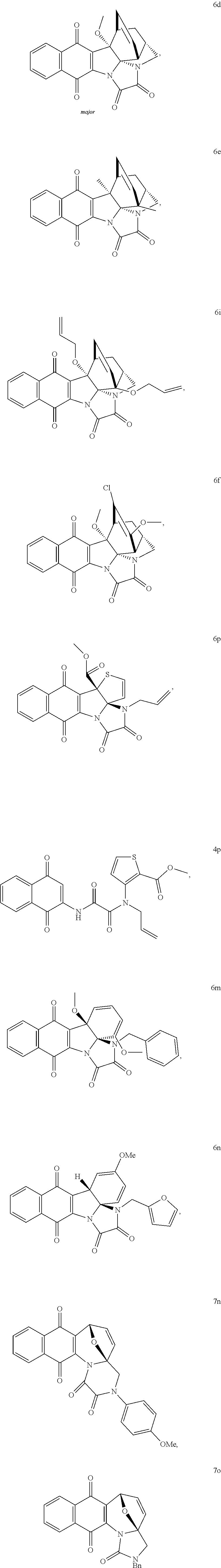

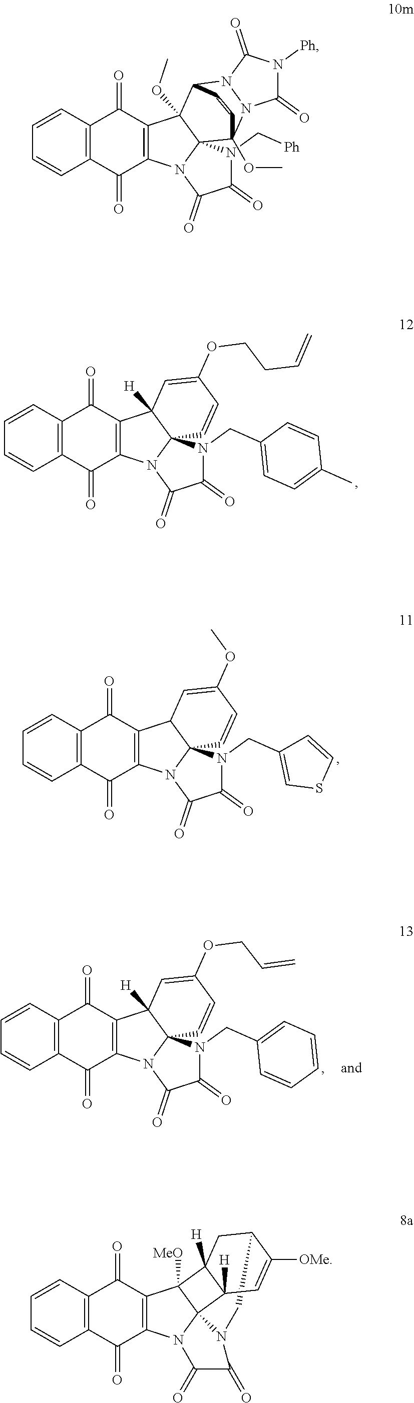

15. The compound of claim 13, wherein the compound is selected from the group consisting of:

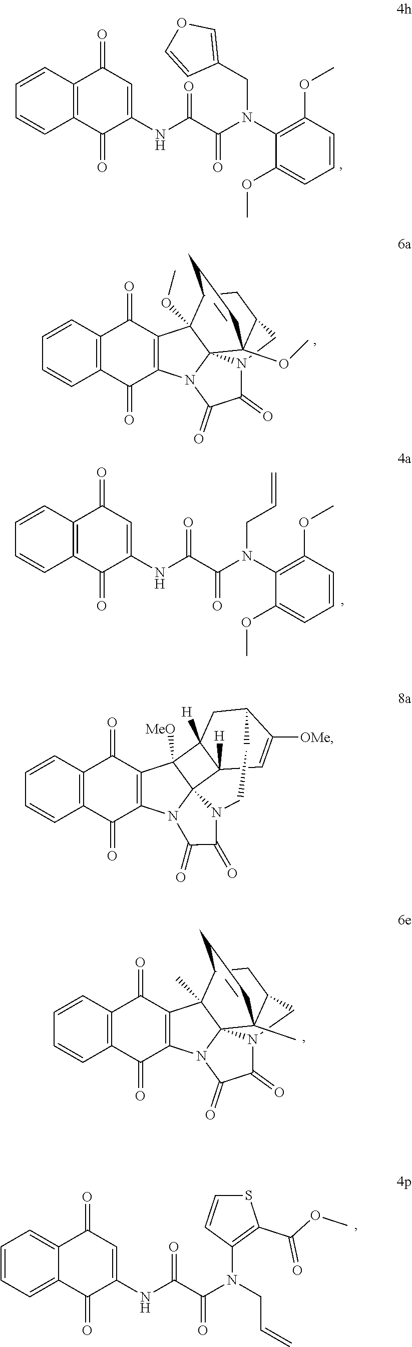

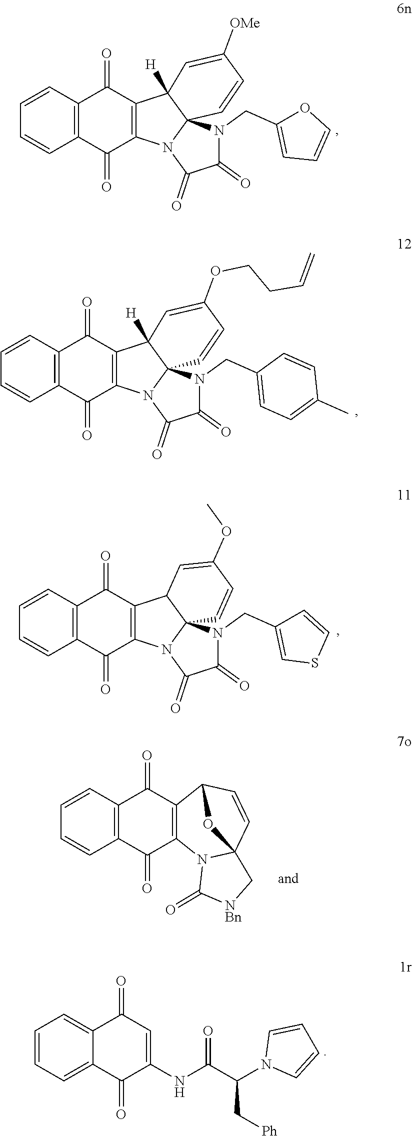

16. A method of treating cancer comprising providing an effective amount of a compound or a pharmaceutically acceptable salt thereof, wherein the compound is selected from the group consisting of:

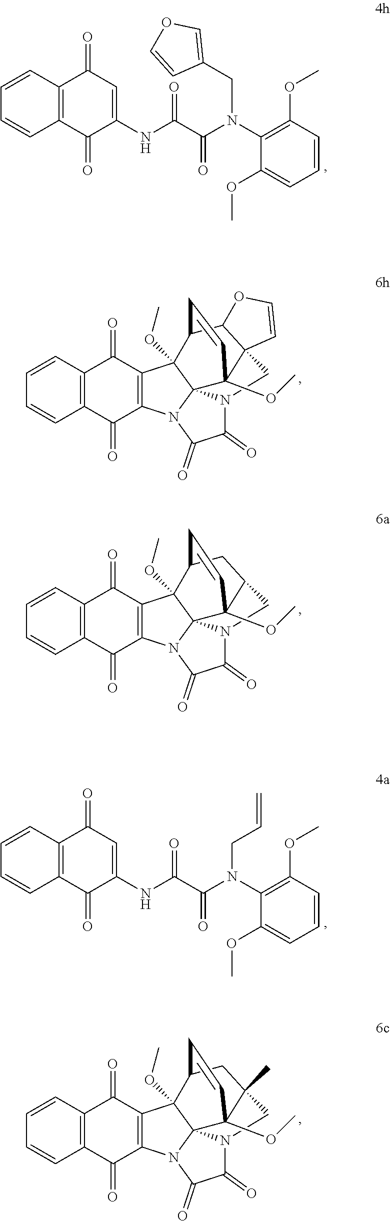 [SMILES: COc1cccc(OC)c1N(Cc1ccoc1)C(=O)C(=O)NC1=CC(=O)c2ccccc2C1=O ; CO[C@@]12C=C[C@H]3C4OC=C[C@]41CN1C(=O)C(=O)N4C5=C(C(=O)c6ccccc6C5=O)[C@]3(OC)[C@]142 ; CO[C@@]12C=C[C@H]3C[C@@H]1CN1C(=O)C(=O)N4C5=C(C(=O)c6ccccc6C5=O)[C@]3(OC)[C@]142 ; C=CCN(C(=O)C(=O)NC1=CC(=O)c2ccccc2C1=O)c1c(OC)cccc1OC ; CO[C@@]12C=C[C@H]3C[C@]1(C)CN1C(=O)C(=O)N4C5=C(C(=O)c6ccccc6C5=O)[C@]3(OC)[C@]142]

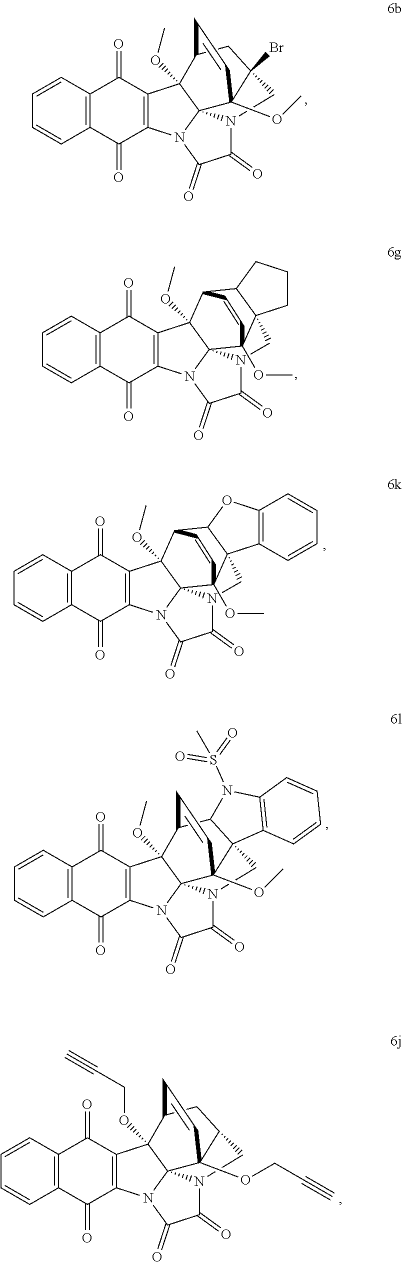 [SMILES: CO[C@@]12C=C[C@H]3C[C@]1(Br)CN1C(=O)C(=O)N4C5=C(C(=O)c6ccccc6C5=O)[C@]3(OC)[C@]142 ; CO[C@@]12C=C[C@H]3C4CCC[C@]41CN1C(=O)C(=O)N4C5=C(C(=O)c6ccccc6C5=O)[C@]3(OC)[C@]142 ; CO[C@@]12C=C[C@H]3C4Oc5ccccc5[C@]41CN1C(=O)C(=O)N4C5=C(C(=O)c6ccccc6C5=O)[C@]3(OC)[C@]142 ; CO[C@@]12C=C[C@H]3C4N(S(C)(=O)=O)c5ccccc5[C@]41CN1C(=O)C(=O)N4C5=C(C(=O)c6ccccc6C5=O)[C@]3(OC)[C@]142 ; C#CCO[C@@]12C=C[C@H]3C[C@@H]1CN1C(=O)C(=O)N4C5=C(C(=O)c6ccccc6C5=O)[C@]3(OCC#C)[C@]142]

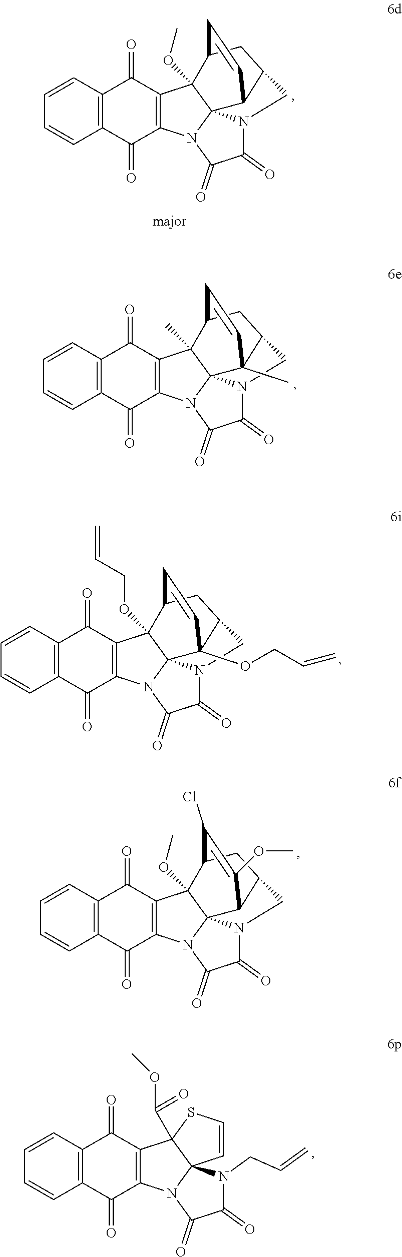 [SMILES: CO[C@]12C3=C(C(=O)c4ccccc4C3=O)N3C(=O)C(=O)N4C[C@H]5C[C@@H]1C=C[C@H]5[C@@]432 ; C[C@]12C3=C(C(=O)c4ccccc4C3=O)N3C(=O)C(=O)N4C[C@H]5C[C@@H]1C=C[C@@]5(C)[C@@]432 ; C=CCO[C@@]12C=C[C@H]3C[C@@H]1CN1C(=O)C(=O)N4C5=C(C(=O)c6ccccc6C5=O)[C@]3(OCC=C)[C@]142 ; COC1=C(Cl)[C@H]2C[C@@H]3CN4C(=O)C(=O)N5C6=C(C(=O)c7ccccc7C6=O)[C@]2(OC)[C@]45[C@H]13 ; C=CCN1C(=O)C(=O)N2C3=C(C(=O)c4ccccc4C3=O)C3(C(=O)OC)SC=C[C@]123]

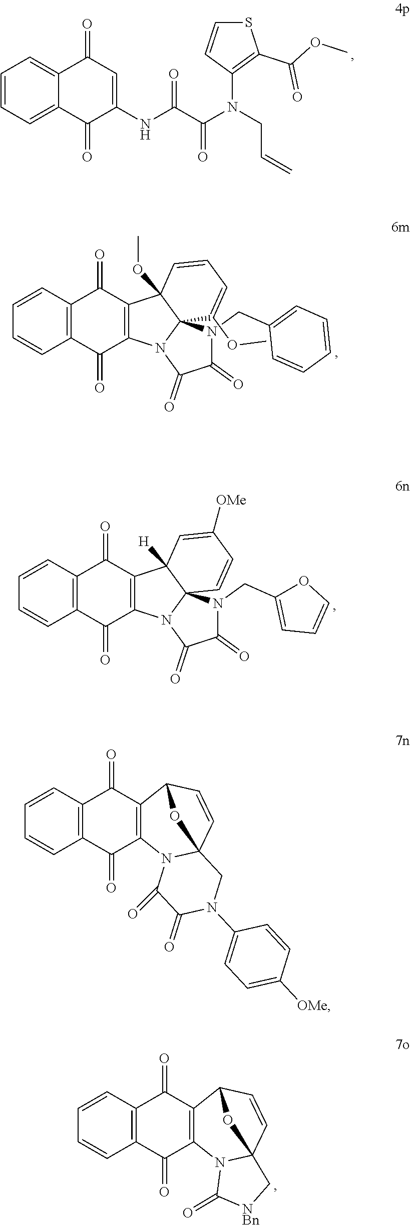 [SMILES: C=CCN(C(=O)C(=O)NC1=CC(=O)c2ccccc2C1=O)c1ccsc1C(=O)OC ; COC1=CC=C[C@@]2(OC)C3=C(C(=O)c4ccccc4C3=O)N3C(=O)C(=O)N(Cc4ccccc4)[C@@]132 ; COC1=C[C@H]2C3=C(C(=O)c4ccccc4C3=O)N3C(=O)C(=O)N(Cc4ccco4)[C@]23C=C1 ; COc1ccc(N2C[C@]34C=C[C@H](O3)C3=C(C(=O)c5ccccc5C3=O)N4C(=O)C2=O)cc1 ; O=C1C2=C(C(=O)c3ccccc31)N1C(=O)N(Cc3ccccc3)C[C@@]13C=C[C@@H]2O3]

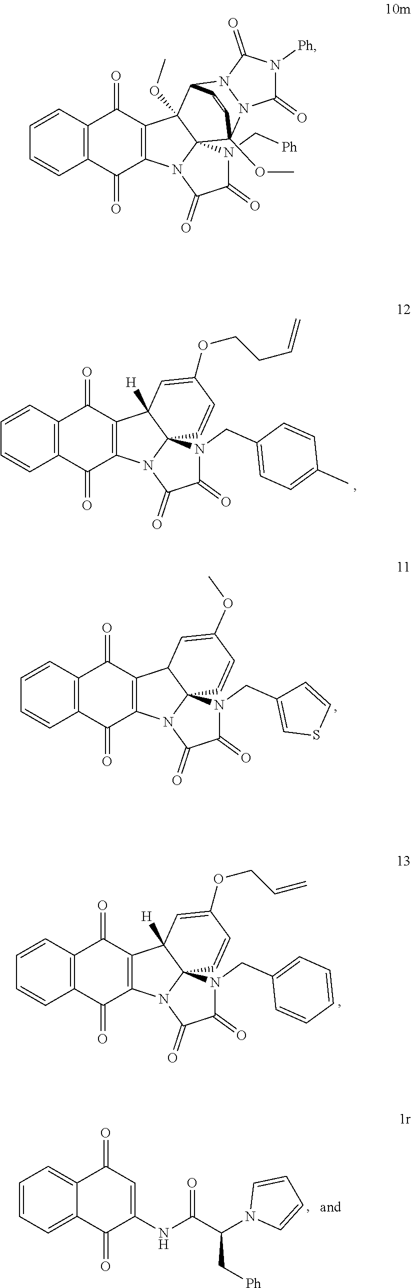

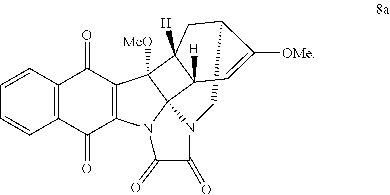 [SMILES: COC1=C[C@H]2[C@H]3C[C@@H]1CN1C(=O)C(=O)N4C5=C(C(=O)c6ccccc6C5=O)[C@]3(OC)[C@]214]

17. The method of claim 16, wherein the cancer is selected from leukemia, non-small cell lung cancer, colon cancer, CNS cancer, melanoma, ovarian cancer, renal cancer, prostate cancer, and breast cancer.

18. The method of claim 16, wherein the cancer is selected from leukemia, non-small cell lung cancer, melanoma, ovarian cancer, prostate cancer, and breast cancer.

19. The method of claim 16, wherein the compound is 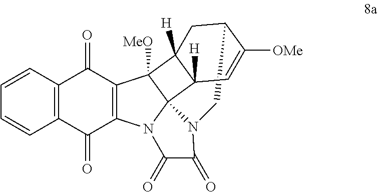 [SMILES: COC1=C[C@H]2[C@H]3C[C@@H]1CN1C(=O)C(=O)N4C5=C(C(=O)c6ccccc6C5=O)[C@]3(OC)[C@]214]
and the cancer is selected from non-small cell lung cancer, CNS cancer, melanoma, ovarian cancer, and breast cancer.

20. The method of claim 19, wherein the cancer is leukemia.
